(19) United States
(12) Patent Application Publication
(10) Pub. No.: US 2025/0354998 A1
(43) Pub. Date: Nov. 20, 2025

(54) METHODS OF DIAGNOSING AND TREATING MULTIPLE SCLEROSIS BY DETECTING A BIOMARKER IN THE CEREBROSPINAL FLUID

(71) Applicants: Principia Biopharma Inc., Morristown, NJ (US); The United States Of America, As Represented By The Secretary, Department Of Health And Human Servic, Bethesda, MD (US)

(72) Inventors: Anna Blazier, Wayland, MA (US); Maria Ines Gaitan, Bethesda, MD (US); Steven Jacobsen, Rockville, MD (US); Dimitry Ofengeim, Dedham, MA (US); Syed Ali Raza, Bethesda, MD (US); Daniel S. Reich, Washington, DC (US); Timothy J. Turner, Belmont, MA (US); Gregory Wirak, Boston, MA (US)

(73) Assignees: Principia Biopharma Inc., Morristown, NJ (US); The United States Of America, As Represented By The Secretary, Department Of Health And Human Servic, Bethesda, MD (US)

(21) Appl. No.: 19/285,681

(22) Filed: Jul. 30, 2025

Related U.S. Application Data
(63) Continuation of application No. PCT/US24/13658, filed on Jan. 31, 2024.
(60) Provisional application No. 63/482,714, filed on Feb. 1, 2023.
(60) Provisional application No. 63/623,681, filed on Jan. 22, 2024.

(51) Int. Cl. G01N 33/68; A61K 31/4545

(52) U.S. Cl. CPC G01N 33/6863; A61K 31/4545; G01N 2333/521; G01N 2333/70578; G01N 2800/285; G01N 2800/50; G01N 2800/52

(57) ABSTRACT

Provided herein are methods and immune biomarkers that identify progression and treatment options for multiple sclerosis (MS). Also provided are materials and methods for the prognosis, staging, and monitoring of MS in a sample and include methods of determining a subject as being at risk of developing MS. The methods include detecting at least one biomarker, such as CXCL13, CXCL10, CD27, NEFL, CCL4, and/or CCL3 in cerebrospinal fluid (CSF) in the subject; and administering a pharmaceutically effective amount of a treatment (e.g., tolebrutinib and potentially one or more additional therapies, such as an anti-CD20 therapy) to the subject.

### CROSS-REFERENCE TO RELATED APPLICATIONS

This application claims the benefit of priority of U.S. Provisional Application No. 63/482,714, filed Feb. 1, 2023, and U.S. Provisional Application No. 63/623,681, filed Jan. 22, 2024, which are incorporated by reference herein in their entirety for any purpose.

### GOVERNMENT INTERESTS

### Parties to a Joint Research Agreement

This invention is a jointly made subject invention under the Public Health Service Cooperative Research and Development Agreement (PHS-CRADA Ref. No. 2020-0226) between the National Institute of Neurological Disorders and Stroke (NINDS) at the National Institutes of Health and Sanofi-Genzyme. The Government of the United States of America has certain rights in this invention.

### REFERENCE TO ELECTRONIC SEQUENCE LISTING

The application contains a Sequence Listing which has been submitted electronically in .XML format and is hereby incorporated by reference in its entirety. Said .XML copy, created on Jan. 29, 2024, is named “01183-0270-00PCT-PRN.xml” and is 19,427 bytes in size. The sequence listing contained in this .XML file is part of the specification and is hereby incorporated by reference herein in its entirety.

### TECHNICAL FIELD

This disclosure relates generally to the screening, detection, prognosis, and treatment of subjects having multiple sclerosis by detecting one or more biomarkers-including CXCL13, CXCL10, CD27, NEFL, CCL4, and/or CCL3-in cerebrospinal fluid (CSF) of a patient.

### BACKGROUND

Multiple Sclerosis (MS) is a neurological disease affecting more than 1 million people worldwide. It is the most common cause of neurological disability in young and middle-aged adults and has a major physical, psychological, social, and financial impact on subjects and their families. MS involves an immune-mediated process in which an abnormal response of the body's immune system is directed against the central nervous system (CNS). In the course of the disease, scleroses, i.e., lesions or scars, appear in the myelin sheath of nerve cells, disrupting transmission of electrical signals. Scleroses accumulate over time and result in the debilitating symptoms experienced by MS patients. MS patients generally experience one of four clinical courses of disease, each of which might be mild, moderate, or severe: clinically isolated syndrome, relapsing remitting, secondary progressive and primary progressive. About 85% of MS patients have the relapsing remitting form of the disease, in which they experience clearly defined relapses (also called flare-ups or exacerbations), which are episodes of acute worsening of neurologic function, followed by partial or complete recovery periods (remissions) that are free of disease progression.

Therapies such as tolebrutinib have been developed. See e.g., U.S. Pat. No. 9,688,676, U.S. Publ. No. 2021/0244720, and PCT Publ. No. WO 2022/140511, each of which is incorporated by reference in its entirety. With the inclusion of various therapies, it is necessary to understand the efficacy of such treatments. Thus, molecular biomarkers are needed to measure MS disease activity and to evaluate therapeutic efficacy. Thus, in order to diagnose and monitor MS progression and severity, it is necessary to develop sensitive molecular tests to identify and measure molecular biomarkers associated with MS.

### SUMMARY

The present disclosure has identified that proteins measured in the cerebrospinal fluid (CSF) may serve as a window into neuroinflammation and can be used to evaluate disease course prognosis, as well as help provide evidence of treatment response following therapy. In the foregoing examples, the present disclosure demonstrates that Olink Proteomics can be used to evaluate proteome expression through a high throughput, multiplex immunoassay technology that enables the measurement of over 1000 proteins from small sample volumes. This type of assay can be performed on a variety of subjects, including those who are undergoing therapy, those whose therapy is to be determined, and those who are monitored for development of multiple sclerosis. Physiological improvements and impairments such as changes in lesions (e.g., active lesions as measured by Magnetic resonance imaging (MRI)) can be monitored concurrently to evaluate the correlation between proteome expression in the CSF and clinical outcome. In addition, therapeutic treatments can be altered and/or examined to determined efficacy. For instances, in some instances disclosed herein, alterations to the MS CSF proteome upon therapeutic intervention with either ocrelizumab, a B cell depleting agent, or with tolebrutinib, a brain penetrant Bruton's tyrosine kinase (BTK) inhibitor, can be evaluated. In doing so, one can gain insights into both disease pathophysiology and the effects of therapeutic intervention.

The present disclosure relates to detection of at least one biomarker to predict or confirm a treatment response for MS, wherein the at least one biomarker is chosen from the proteins listed in Tables 1-3, such as CXCL13, CXCL10, CD27, NEFL, CCL4, and CCL3. In some instances, the at least one biomarker is chosen from the proteins listed in Table 3. In some instances, the present disclosure relates to detection of at least 2 biomarkers, at least 3 biomarkers, at least 4 biomarkers, at least 5 biomarkers, at least 6 biomarkers, or more to predict or confirm a treatment response for MS. The present disclosure has also identified that certain treatments can result in changes of expression of at least one, at least two, at least three, at least four, at least five, at least six, or more biomarkers. In particular, one instance disclosed herein includes a treatment change (e.g., from an anti-CD20 antibody such as ocrelizumab (Ocrevus®) to tolebrutinib). The present disclosure has identified that the biomarker(s) disclosed herein can be used for prognosis, development, and therapeutic efficacy in the cerebrospinal fluid (CSF) of a subject with MS. Modulation of the biomarker(s) (e.g., a decrease in biomarker(s)) protein expression is associated with more favorable outcomes of MS. Thus, provided herein are methods of detecting at least one, at least two, at least three, at least four, at least five, at least six, or more biomarkers (both RNA and protein), which can indicate severity of MS, including the progression, regression, or static presence of lesions (e.g., active lesions) in the brain of a subject with MS.

Thus, in one embodiment, provided herein is a method of treating a subject having MS, the method comprising: (a) detecting at least one biomarker in cerebrospinal fluid (CSF) in the subject; and (b) administering a pharmaceutically effective amount of tolebrutinib to the subject. In a second aspect, disclosed herein is a method of treating a subject having MS, the method comprising: (a) detecting at least one biomarker in a biological sample comprising CSF from the subject; (b) identifying the subject expressing the at least one biomarker in the biological sample as having MS; and (c) administering a pharmaceutically effective amount of tolebrutinib to the subject. In some instances, the methods include detecting at least two biomarkers, at least three biomarkers, at least four biomarkers, at least five biomarkers, at least six biomarkers, or more. In some instances, the pharmaceutically effective amount of tolebrutinib is at a dose of about 50 mg to about 130 mg. In some instances, the pharmaceutically effective amount of tolebrutinib is at a dose of 60 mg. In some instances, the pharmaceutically effective amount of tolebrutinib is at a dose of 120 mg. In some instances, the pharmaceutically effective amount of tolebrutinib is administered orally. In some instances, the pharmaceutically effective amount of tolebrutinib is administered daily. In some instances, the method of treating a subject having MS further comprises administering a pharmaceutically effective amount of an anti-CD20 antibody. In some instances, the anti-CD20 antibody comprises ocrelizumab or rituximab. In some instances, the initial anti-CD20 antibody dose comprises one or more intravenous infusion (e.g., intravenous (IV) infusion) of an anti-CD20 antibody. The one or more IV infusions can be administered over a period of time (e.g., for at least six months). In some instances, the subject is administered the IV infusion at doses that are about a week to about three weeks (e.g., about 1 week, about 2 weeks, or about 3 weeks) apart. In some instances, the anti-CD20 antibody is administered during treatment with tolebrutinib. In some instances, the anti-CD20 antibody is administered before treatment with tolebrutinib. In some instances, the anti-CD20 antibody is administered after treatment with tolebrutinib.

In another embodiment, disclosed herein is a method of identifying a patient as being suitable for participating in a clinical trial for MS, the method comprising: (a) detecting at least one biomarker in a biological sample comprising CSF from the subject; and (b) identifying the subject expressing the at least one biomarker in the biological sample, thereby identifying a patient as being suitable for participating in a clinical trial for MS. In some instances, the methods comprise detecting at least two biomarkers, at least three biomarkers, at least four biomarkers, at least five biomarkers, at least six biomarkers, or more.

In yet another embodiment, disclosed herein is a method of diagnosing a subject as having MS, the method comprising: (a) detecting at least one biomarker in a biological sample comprising CSF from the subject; and (b) identifying the subject expressing the at least one biomarker in the biological sample as having MS. In some instances, the methods comprise detecting at least two biomarkers, at least three biomarkers, at least four biomarkers, at least five biomarkers, at least six biomarkers, or more.

In yet another embodiment, disclosed herein is a method of identifying a subject having MS as expressing at least one biomarker in a biological sample comprising CSF, the method comprising: (a) detecting at least one biomarker in the biological sample; and (b) identifying the subject having MS expressing the at least one biomarker in the biological sample. In some instances, the methods comprise identifying a subject having MS as expressing at least two biomarkers, at least three biomarkers, at least four biomarkers, at least five biomarkers, at least six biomarkers, or more.

Also disclosed herein is a method of identifying a subject as having an increased likelihood of developing MS, the method comprising: (a) detecting at least one biomarker in a biological sample comprising CSF from a subject; and (b) identifying a subject expressing at least one biomarker in the biological sample, as having an increased likelihood of developing MS. In some instances, the method comprises detecting at least two biomarkers, at least three biomarkers, at least four biomarkers, at least five biomarkers, at least six biomarkers, or more.

In another embodiment, disclosed herein is a method of identifying a subject as likely to develop MS, the method comprising: (a) detecting at least one biomarker in a biological sample comprising CSF from a subject; and (b) identifying a subject expressing the at least one biomarker in the biological sample, as having an increased likelihood of developing MS. In some instances, the method comprises detecting at least two biomarkers, at least three biomarkers, at least four biomarkers, at least five biomarkers, at least six biomarkers, or more.

In some instances, the biomarker(s) is increased compared to the biomarker(s) in a reference sample. In some instances, the reference sample is from a second subject. In some instances, the second subject does not have MS. In some instances, the reference sample comprises CSF. In some instances, the method further includes obtaining the biological sample from the subject.

In some instances, the disclosure also provides a method of monitoring progression of MS in a subject over time, the method comprising: (a) detecting at least one biomarker in a first biological sample obtained from a subject at a first time point; (b) detecting the at least one biomarker in a second biological sample obtained from the subject at a second time point; and (c) identifying: (i) a subject having increased at least one biomarker at the second time point, as compared to the first time point, as having progressing MS, or (ii) a subject having about the same or a decreased at least one biomarker at the second time point, as compared to the first time point, as having static or regressing MS. In some instances, the method comprises detecting at least two biomarkers, at least three biomarkers, at least four biomarkers, at least five biomarkers, at least six biomarkers, or more, and comparing the biomarkers.

In some instances, the method includes administering a pharmaceutically effective amount of tolebrutinib to the subject. In some instances, the pharmaceutically effective amount of tolebrutinib is at a dose of about 50 mg to about 130 mg. In some instances, the pharmaceutically effective amount of tolebrutinib is at a dose of 60 mg. In some instances, the pharmaceutically effective amount of tolebrutinib is at a dose of 120 mg. In some instances, the pharmaceutically effective amount of tolebrutinib is administered orally. In some instances, the pharmaceutically effective amount of tolebrutinib is administered daily. In an embodiment, the method further comprises administering a pharmaceutically effective amount of an anti-CD20 antibody to the subject. In some instances, the initial anti-CD20 antibody dose comprises one or more intravenous infusion (e.g., intravenous (IV) infusion) of an anti-CD20 antibody. The one or more IV infusions can be administered over a period of time (e.g., for at least six months). In some instances the subject is administered the IV infusion at doses that are about a week to about three weeks (e.g., about 1 week, about 2 weeks, or about 3 weeks) apart. In some instances, the anti-CD20 antibody is administered during treatment with tolebrutinib. In some instances, the anti-CD20 antibody is administered before treatment with tolebrutinib. In some instances, the anti-CD20 antibody is administered after treatment with tolebrutinib.

In some instances, the methods disclosed herein include a method of assessing the efficacy of a treatment of a pharmaceutically effective amount of tolebrutinib in a subject having MS, the method comprising: (a) detecting (i) at least one biomarker in a first biological sample comprising CSF obtained from the subject at a first time point and (ii) the at least one biomarker in a second biological sample comprising CSF obtained from the subject at a second time point, wherein the subject is administered one or more doses of the treatment between the first and second time points; and (b) determining a correlation between efficacy of the treatment and the at least one biomarker in the second biological sample as compared to the at least one biomarker in a sample obtained from an untreated patient, wherein the at least one biomarker in the second biological sample is about the same or decreased as compared to the abundance in the sample from the untreated patient, thereby indicating that the treatment is effective for MS in the subject. In some instances, the method comprises detecting at least two biomarkers, at least three biomarkers, at least four biomarkers, at least five biomarkers, at least six biomarkers, or more, and determining the correlation between efficacy of treatment and the biomarker(s). In an embodiment, the method further comprises administering a pharmaceutically effective amount of an anti-CD20 antibody to the subject and assessing the efficacy of a treatment of a pharmaceutically effective amount of tolebrutinib and anti-CD20 antibody.

In some instances, the difference between the first time point and the second time point is about 1 month to about two years. In some instances, the pharmaceutically effective amount of tolebrutinib is administered at a dose of about 50 mg to about 130 mg. In some instances, the pharmaceutically effective amount of tolebrutinib is at a dose of 60 mg. In some instances, the pharmaceutically effective amount of tolebrutinib is at a dose of 120 mg. In some instances, the pharmaceutically effective amount of tolebrutinib is administered orally. In some instances, the pharmaceutically effective amount of tolebrutinib is administered daily. In some instances, the anti-CD20 antibody dose comprises one or more intravenous infusion (e.g., intravenous (IV) infusion) of an anti-CD20 antibody. The one or more IV infusions can be administered over a period of time (e.g., for at least six months). In some instances, the subject is administered the IV infusion at doses that are about a week to about three weeks (e.g., about 1 week, about 2 weeks, or about 3 weeks) apart. In some instances, the at least one biomarker comprises RNA. In some instances, the at least one biomarker RNA is determined by polymerase chain reaction, quantitative polymerase chain reaction, or Northern blot. In some instances, the at least one biomarker comprises protein. In some instances, the at least one biomarker is determined by flow cytometry or Western blot. In some instances, the method further comprises monitoring the subject for the development of symptoms of MS. In some instances, the method further comprises administering to the subject a treatment for decreasing the rate of progression or decreasing the likelihood or susceptibility of developing MS.

In some instances, the at least one biomarker is chosen from the proteins listed in Tables 1-3. In some instances, the at least one biomarker is chosen from the proteins listed in Table 3. In some instances, the at least one biomarker is chosen from CXCL13, CXCL10, CD27, NEFL, CCL4, and CCL3.

In some instances, the preceding methods include administering additional or increased doses of the pharmaceutically effective amount of tolebrutinib for the subject. In some instances, the preceding methods include administering additional or increased doses of the pharmaceutically effective amount of tolebrutinib and an anti-CD20 antibody for the subject.

In some instances, the subject has received a treatment for MS prior to detecting at least one biomarker. In some instances, the subject has been treated previously with an anti-CD20 therapy.

In some instances, the anti-CD20 therapy is ocrelizumab or rituximab.

In some instances, the subject has not received a treatment for MS prior to detecting at least one biomarker and/or has not been treated previously with an anti-CD20 therapy.

In some instances, the subject comprises one or more brain lesions.

In some instances, the subject is administered one or more doses of an anti-CD20 therapy and/or a booster of an anti-CD20 therapy in addition to the pharmaceutically effective amount of tolebrutinib.

In some instances, administering of the pharmaceutically effective amount of tolebrutinib (and potentially with one or more additional therapies, such as an anti-CD20 therapy) results in one or more of: reduced brain lesions; a reduced amount of iron in brain tissue of the subject; and/or a decrease in synaptic density. In some instances, reduced or decrease(d) is compared to a previous amount from the same subject.

In some instances, the MS is relapsing-remitting multiple sclerosis. In some instances, the MS is secondary progressive multiple sclerosis.

The present disclosure relates to detection of CXCL13 as a biomarker to predict or confirm a treatment response for MS. The present disclosure has also identified that certain treatments can result in changes of CXCL13 biomarker expression. In particular, one embodiment disclosed herein includes a treatment change (e.g., from ocrelizumab (Ocrevus®) to tolebrutinib). The present disclosure has identified that CXCL13 can be used as a biomarker of prognosis, development, and therapeutic efficacy in the cerebrospinal fluid (CSF) of a subject with MS. Modulation of CXCL13 (e.g., a decrease in CXCL13) protein expression is associated with more favorable outcomes of MS. Thus, provided herein are methods of detecting CXCL13 (both RNA and protein) as a biomarker that indicates severity of MS, including the progression, regression, or static presence of lesions (e.g., active lesions) in the brain of a subject with MS.

Thus, in one aspect, provided herein a method of treating a subject having MS, the method comprising: (a) detecting CXCL13 in cerebrospinal fluid (CSF) in the subject; and (b) administering a pharmaceutically effective amount of tolebrutinib to the subject. In a second aspect, disclosed herein is a method of treating a subject having MS, the method comprising: (a) detecting CXCL13 in a biological sample comprising CSF from the subject; (b) identifying the subject expressing CXCL13 in the biological sample as having MS; and (c) administering a pharmaceutically effective amount of tolebrutinib to the subject. In some instances, the pharmaceutically effective amount of tolebrutinib is at a dose of about 50 mg to about 130 mg. In some instances, the pharmaceutically effective amount of tolebrutinib is at a dose of 60 mg. In some instances, the pharmaceutically effective amount of tolebrutinib is at a dose of 120 mg. In some instances, the pharmaceutically effective amount of tolebrutinib is administered orally. In some instances, the pharmaceutically effective amount of tolebrutinib is administered daily. In an embodiment, the method of treating a subject having MS further comprises administering a pharmaceutically effective amount of an anti-CD20 antibody. In some instances, the anti-CD20 antibody comprises ocrelizumab or rituximab. In some instances, the anti-CD20 antibody dose comprises one or more intravenous infusion (e.g., intravenous (IV) infusion) of an anti-CD20 antibody. The one or more IV infusions can be administered over a period of time (e.g., for at least six months). In some instances, the subject is administered the IV infusion at doses that are about a week to about three weeks (e.g., about 1 week, about 2 weeks, or about 3 weeks) apart. In some instances, the anti-CD20 antibody is administered during treatment with tolebrutinib. In some instances, the anti-CD20 antibody is administered separately from treatment with tolebrutinib. In some instances, the anti-CD20 antibody is administered before treatment with tolebrutinib. In some instances the anti-CD20 antibody is administered after treatment with tolebrutinib.

In another embodiment, disclosed herein is a method of identifying a patient as being suitable for participating in a clinical trial for MS, the method comprising: (a) detecting CXCL13 in a biological sample comprising CSF from the subject; and (b) identifying the subject expressing CXCL13 in the biological sample, thereby identifying a patient as being suitable for participating in a clinical trial for MS.

In yet another embodiment, disclosed herein is a method of diagnosing a subject as having MS, the method comprising: (a) detecting CXCL13 in a biological sample comprising CSF from the subject; and (b) identifying the subject expressing CXCL13 in the biological sample as having MS.

In yet another embodiment, disclosed herein is a method of identifying a subject having MS as expressing CXCL13 in a biological sample comprising CSF, the method comprising: (a) detecting CXCL13 in the biological sample; and (b) identifying the subject having MS expressing CXCL13 in the biological sample.

Also disclosed herein is a method of identifying a subject as having an increased likelihood of developing MS, the method comprising: (a) detecting CXCL13 in a biological sample comprising CSF from a subject; and (b) identifying a subject expressing CXCL13 in the biological sample, as having an increased likelihood of developing MS.

In another embodiment, disclosed herein is a method of identifying a subject as likely to develop MS, the method comprising: (a) detecting CXCL13 in a biological sample comprising CSF from a subject; and (b) identifying a subject expressing CXCL13 in the biological sample, as having an increased likelihood of developing MS.

In some instances, the CXCL13 is increased compared to CXCL13 in a reference sample. In some instances, the reference sample is from a second subject. In some instances, the second subject does not have MS. In some instances, the reference sample comprises CSF. In some instances, the method further includes obtaining the biological sample from the subject.

In some instances, the disclosure also provides a method of monitoring progression of MS in a subject over time, the method comprising: (a) detecting CXCL13 in a first biological sample obtained from a subject at a first time point; (b) detecting CXCL13 in a second biological sample obtained from the subject at a second time point; and (c) identifying: (i) a subject having increased CXCL13 at the second time point, as compared to CXCL13 at the first time point, as having progressing MS, or (ii) a subject having about the same or a decreased CXCL13 at the second time point, as compared to CXCL13 at the first time point, as having static or regressing MS.

In some instances, the method includes administering a pharmaceutically effective amount of tolebrutinib to the subject. In some instances, the pharmaceutically effective amount of tolebrutinib is at a dose of about 50 mg to about 130 mg. In some instances, the pharmaceutically effective amount of tolebrutinib is at a dose of 60 mg. In some instances, the pharmaceutically effective amount of tolebrutinib is at a dose of 120 mg. In some instances, the pharmaceutically effective amount of tolebrutinib is administered orally. In some instances, the pharmaceutically effective amount of tolebrutinib is administered daily. In an embodiment, the method further comprises administering a pharmaceutically effective amount of an anti-CD20 antibody to the subject. In some instances, the anti-CD20 antibody dose comprises one or more intravenous infusion (e.g., intravenous (IV) infusion) of an anti-CD20 antibody. The one or more IV infusions can be administered over a period of time (e.g., for at least six months). In some instances the subject is administered the IV infusion at doses that are about a week to about three weeks (e.g., about 1 week, about 2 weeks, or about 3 weeks) apart. In some instances, the anti-CD20 antibody is administered during treatment with tolebrutinib. In some instances, the anti-CD20 antibody is administered separately from treatment with tolebrutinib. In some instances, the anti-CD20 antibody is administered before treatment with tolebrutinib. In some instances the anti-CD20 antibody is administered after treatment with tolebrutinib

In some instances, the methods disclosed herein include a method of assessing the efficacy of a treatment of a pharmaceutically effective amount of tolebrutinib in a subject having MS, the method comprising: (a) detecting (i) CXCL13 in a first biological sample comprising CSF obtained from the subject at a first time point and (ii) CXCL13 in a second biological sample comprising CSF obtained from the subject at a second time point, wherein the subject is administered one or more doses of the treatment between the first and second time points; and (b) determining a correlation between efficacy of the treatment and CXCL13 in the second biological sample as compared to CXCL13 in a sample obtained from an untreated patient, wherein the CXCL13 in the second biological sample is about the same or decreased as compared to the abundance in the sample from the untreated patient, thereby indicating that the treatment is effective for MS in the subject. In an embodiment, the method further comprises administering a pharmaceutically effective amount of an anti-CD20 antibody to the subject and assessing the efficacy of a treatment of a pharmaceutically effective amount of tolebrutinib and anti-CD20 antibody.

In some instances, the difference between the first time point and the second time point is about 1 month to about two years. In some instances, the pharmaceutically effective amount of tolebrutinib is administered at a dose of about 50 mg to about 130 mg. In some instances, the pharmaceutically effective amount of tolebrutinib is at a dose of 60 mg. In some instances, the pharmaceutically effective amount of tolebrutinib is at a dose of 120 mg. In some instances, the pharmaceutically effective amount of tolebrutinib is administered orally. In some instances, the pharmaceutically effective amount of tolebrutinib is administered daily. In some instances, the anti-CD20 antibody dose comprises one or more intravenous infusion (e.g., intravenous (IV) infusion) of an anti-CD20 antibody. The one or more IV infusions can be administered over a period of time (e.g., for at least six months). In some instances, the subject is administered the IV infusion at doses that are about a week to about three weeks (e.g., about 1 week, about 2 weeks, or about 3 weeks) apart. In some instances, the CXCL13 comprises CXCL13 RNA. In some instances, the CXCL13 RNA is determined by polymerase chain reaction, quantitative polymerase chain reaction, or Northern blot. In some instances, the CXCL13 comprises CXCL13 protein. In some instances, the CXCL13 is determined by flow cytometry or Western blot. In some instances, the method further comprises monitoring the subject for the development of symptoms of MS. In some instances, the method further comprises administering to the subject a treatment for decreasing the rate of progression or decreasing the likelihood or susceptibility of developing MS.

In some instances, the preceding methods include administering additional or increased doses of the pharmaceutically effective amount of tolebrutinib for the subject. In some instances, the preceding methods further include administering additional or increased doses of an anti-CD20 antibody for the subject.

In some instances, the subject has received a treatment for MS prior to detecting CXCL13. In some instances, the subject has been treated previously with an anti-CD20 therapy.

In some instances, the anti-CD20 therapy is ocrelizumab. In some instances, the anti-CD20 therapy is rituximab.

In some instances, the subject has not received a treatment for MS prior to detecting CXCL13 and/or has not been treated previously with an anti-CD20 therapy.

In some instances, the subject comprises one or more brain lesions.

In some instances, the subject is administered one or more doses of an anti-CD20 therapy and/or a booster of an anti-CD20 therapy in addition to the pharmaceutically effective amount of tolebrutinib.

In some instances, administering of the pharmaceutically effective amount of tolebrutinib (and potentially with one or more additional therapies, such as an anti-CD20 therapy) results in one or more of: reduced brain lesions; a reduced amount of iron in brain tissue of the subject; and/or a decrease in synaptic density. In some instances, reduced or decrease(d) is compared to a previous amount from the same subject.

In some instances, the MS is relapsing-remitting multiple sclerosis. In some instances, the MS is secondary progressive multiple sclerosis.

The present disclosure relates to detection of CXCL10 as a biomarker to predict or confirm a treatment response for MS. C-X-C motif chemokine ligand 10 (CXCL10) also known as Interferon gamma-induced protein 10 (IP-10) or small-inducible cytokine B10 is an 8.7 kDa protein that in humans is encoded by the CXCL10 gene. Luster et al., Nature. 315 (6021): 672-6 (1985); Luster et al., Proceedings of the National Academy of Sciences of the United States of America. 84 (9): 2868-71 (May 1987). The present disclosure has also identified that certain treatments can result in changes of CXCL10 biomarker expression. In particular, one embodiment disclosed herein includes a treatment change (e.g., from ocrelizumab (Ocrevus®) to tolebrutinib). The present disclosure has identified that CXCL10 can be used as a biomarker of prognosis, development, and therapeutic efficacy in the cerebrospinal fluid (CSF) of a subject with MS. Modulation of CXCL10 (e.g., a decrease in CXCL10) protein expression is associated with more favorable outcomes of MS. Thus, provided herein are methods of detecting CXCL10 (both RNA and protein) as a biomarker that indicates severity of MS, including the progression, regression, or static presence of lesions (e.g., active lesions) in the brain of a subject with MS.

Thus, in one aspect, provided herein a method of treating a subject having MS, the method comprising: (a) detecting CXCL10 in cerebrospinal fluid (CSF) in the subject; and (b) administering a pharmaceutically effective amount of tolebrutinib to the subject. In a second aspect, disclosed herein is a method of treating a subject having MS, the method comprising: (a) detecting CXCL10 in a biological sample comprising CSF from the subject; (b) identifying the subject expressing CXCL10 in the biological sample as having MS; and (c) administering a pharmaceutically effective amount of tolebrutinib to the subject. In some instances, the pharmaceutically effective amount of tolebrutinib is at a dose of about 50 mg to about 130 mg. In some instances, the pharmaceutically effective amount of tolebrutinib is at a dose of 60 mg. In some instances, the pharmaceutically effective amount of tolebrutinib is at a dose of 120 mg. In some instances, the pharmaceutically effective amount of tolebrutinib is administered orally. In some instances, the pharmaceutically effective amount of tolebrutinib is administered daily. In an embodiment, the method of treating a subject having MS further comprises administering a pharmaceutically effective amount of an anti-CD20 antibody. In some instances, the anti-CD20 antibody comprises ocrelizumab or rituximab. In some instances, the anti-CD20 antibody dose comprises one or more intravenous infusion (e.g., intravenous (IV) infusion) of an anti-CD20 antibody. The one or more IV infusions can be administered over a period of time (e.g., for at least six months). In some instances, the subject is administered the IV infusion at doses that are about a week to about three weeks (e.g., about 1 week, about 2 weeks, or about 3 weeks) apart. In some instances, the anti-CD20 antibody is administered during treatment with tolebrutinib. In some instances, the anti-CD20 antibody is administered separately from treatment with tolebrutinib. In some instances, the anti-CD20 antibody is administered before treatment with tolebrutinib. In some instances, the anti-CD20 antibody is administered after treatment with tolebrutinib.

In another embodiment, disclosed herein is a method of identifying a patient as being suitable for participating in a clinical trial for MS, the method comprising: (a) detecting CXCL10 in a biological sample comprising CSF from the subject; and (b) identifying the subject expressing CXCL10 in the biological sample, thereby identifying a patient as being suitable for participating in a clinical trial for MS.

In yet another embodiment, disclosed herein is a method of diagnosing a subject as having MS, the method comprising: (a) detecting CXCL10 in a biological sample comprising CSF from the subject; and (b) identifying the subject expressing CXCL10 in the biological sample as having MS.

In yet another embodiment, disclosed herein is a method of identifying a subject having MS as expressing CXCL10 in a biological sample comprising CSF, the method comprising: (a) detecting CXCL10 in the biological sample; and (b) identifying the subject having MS expressing CXCL10 in the biological sample.

Also disclosed herein is a method of identifying a subject as having an increased likelihood of developing MS, the method comprising: (a) detecting CXCL10 in a biological sample comprising CSF from a subject; and (b) identifying a subject expressing CXCL10 in the biological sample, as having an increased likelihood of developing MS.

In another embodiment, disclosed herein is a method of identifying a subject as likely to develop MS, the method comprising: (a) detecting CXCL10 in a biological sample comprising CSF from a subject; and (b) identifying a subject expressing CXCL10 in the biological sample, as having an increased likelihood of developing MS.

In some instances, the CXCL10 is increased compared to CXCL10 in a reference sample. In some instances, the reference sample is from a second subject. In some instances, the second subject does not have MS. In some instances, the reference sample comprises CSF. In some instances, the method further includes obtaining the biological sample from the subject.

In some instances, the disclosure also provides a method of monitoring progression of MS in a subject over time, the method comprising: (a) detecting CXCL10 in a first biological sample obtained from a subject at a first time point; (b) detecting CXCL10 in a second biological sample obtained from the subject at a second time point; and (c) identifying: (i) a subject having increased CXCL10 at the second time point, as compared to CXCL10 at the first time point, as having progressing MS, or (ii) a subject having about the same or a decreased CXCL10 at the second time point, as compared to CXCL10 at the first time point, as having static or regressing MS.

In some instances, the method includes administering a pharmaceutically effective amount of tolebrutinib to the subject. In some instances, the pharmaceutically effective amount of tolebrutinib is at a dose of about 50 mg to about 130 mg. In some instances, the pharmaceutically effective amount of tolebrutinib is at a dose of 60 mg. In some instances, the pharmaceutically effective amount of tolebrutinib is at a dose of 120 mg. In some instances, the pharmaceutically effective amount of tolebrutinib is administered orally. In some instances, the pharmaceutically effective amount of tolebrutinib is administered daily. In an embodiment, the method further comprises administering a pharmaceutically effective amount of an anti-CD20 antibody to the subject. In some instances, the anti-CD20 antibody dose comprises one or more intravenous infusion (e.g., intravenous (IV) infusion) of an anti-CD20 antibody. The one or more IV infusions can be administered over a period of time (e.g., for at least six months). In some instances the subject is administered the IV infusion at doses that are about a week to about three weeks (e.g., about 1 week, about 2 weeks, or about 3 weeks) apart. In some instances, the anti-CD20 antibody is administered concurrently during treatment with tolebrutinib. In some instances, the anti-CD20 antibody is administered separately from treatment with tolebrutinib. In some instances the anti-CD20 antibody is administered before treatment with tolebrutinib. In some instances the anti-CD20 antibody is administered after treatment with tolebrutinib.

In some instances, the methods disclosed herein include a method of assessing the efficacy of a treatment of a pharmaceutically effective amount of tolebrutinib in a subject having MS, the method comprising: (a) detecting (i) CXCL10 in a first biological sample comprising CSF obtained from the subject at a first time point and (ii) CXCL10 in a second biological sample comprising CSF obtained from the subject at a second time point, wherein the subject is administered one or more doses of the treatment between the first and second time points; and (b) determining a correlation between efficacy of the treatment and CXCL10 in the second biological sample as compared to CXCL10 in a sample obtained from an untreated patient, wherein the CXCL10 in the second biological sample is about the same or decreased as compared to the abundance in the sample from the untreated patient, thereby indicating that the treatment is effective for MS in the subject. In an embodiment, the method further comprises administering a pharmaceutically effective amount of an anti-CD20 antibody to the subject and assessing the efficacy of a treatment of a pharmaceutically effective amount of tolebrutinib and anti-CD20 antibody.

In some instances, the difference between the first time point and the second time point is about 1 month to about two years. In some instances, the pharmaceutically effective amount of tolebrutinib is administered at a dose of about 50 mg to about 130 mg. In some instances, the pharmaceutically effective amount of tolebrutinib is at a dose of 60 mg. In some instances, the pharmaceutically effective amount of tolebrutinib is at a dose of 120 mg. In some instances, the pharmaceutically effective amount of tolebrutinib is administered orally. In some instances, the pharmaceutically effective amount of tolebrutinib is administered daily. In some instances, the anti-CD20 antibody dose comprises one or more intravenous infusion (e.g., intravenous (IV) infusion) of an anti-CD20 antibody. The one or more IV infusions can be administered over a period of time (e.g., for at least six months). In some instances, the subject is administered the IV infusion at doses that are about a week to about three weeks (e.g., about 1 week, about 2 weeks, or about 3 weeks) apart. In some instances, the CXCL10 comprises CXCL10 RNA. In some instances, the CXCL10 RNA is determined by polymerase chain reaction, quantitative polymerase chain reaction, or Northern blot. In some instances, the CXCL10 comprises CXCL10 protein. In some instances, the CXCL10 is determined by flow cytometry or Western blot. In some instances, the method further comprises monitoring the subject for the development of symptoms of MS. In some instances, the method further comprises administering to the subject a treatment for decreasing the rate of progression or decreasing the likelihood or susceptibility of developing MS.

In some instances, the preceding methods include administering additional or increased doses of the pharmaceutically effective amount of tolebrutinib for the subject. In some instances, the preceding methods include administering additional or increased doses of the pharmaceutically effective amount of tolebrutinib and anti-CD20 antibody for the subject.

In some instances, the subject has received a treatment for MS prior to detecting CXCL10. In some instances, the subject has been treated previously with an anti-CD20 therapy.

In some instances, the anti-CD20 therapy is ocrelizumab. In some instances, the anti-CD20 therapy is rituximab.

In some instances, the subject comprises one or more brain lesions.

In some instances, the subject is administered one or more doses of an anti-CD20 therapy and/or a booster of an anti-CD20 therapy in addition to the pharmaceutically effective amount of tolebrutinib.

In some instances, administering of the pharmaceutically effective amount of tolebrutinib (and potentially with one or more additional therapies, such as an anti-CD20 therapy) results in one or more of: reduced brain lesions; a reduced amount of iron in brain tissue of the subject; and/or a decrease in synaptic density. In some instances, reduced or decrease(d) is compared to a previous amount from the same subject.

In some instances, the MS is relapsing-remitting multiple sclerosis. In some instances, the MS is secondary progressive multiple sclerosis.

The present disclosure relates to detection of CD27 as a biomarker to predict or confirm a treatment response for MS. The present disclosure has also identified that certain treatments can result in changes of CD27 biomarker expression. In particular, one embodiment disclosed herein includes a treatment change (e.g., from ocrelizumab (Ocrevus®) to tolebrutinib). The present disclosure has identified that CD27 can be used as a biomarker of prognosis, development, and therapeutic efficacy in the cerebrospinal fluid (CSF) of a subject with MS. Modulation of CD27 (e.g., a decrease in CD27 protein expression is associated with more favorable outcomes of MS. Thus, provided herein are methods of detecting CD27 (both RNA and protein) as a biomarker that indicates severity of MS, including the progression, regression, or static presence of lesions (e.g., active lesions) in the brain of a subject with MS.

Thus, in one aspect, provided herein a method of treating a subject having MS, the method comprising: (a) detecting CD27 in cerebrospinal fluid (CSF) in the subject; and (b) administering a pharmaceutically effective amount of tolebrutinib to the subject. In a second aspect, disclosed herein is a method of treating a subject having MS, the method comprising: (a) detecting CD27 in a biological sample comprising CSF from the subject; (b) identifying the subject expressing CD27 in the biological sample as having MS; and (c) administering a pharmaceutically effective amount of tolebrutinib to the subject. In some instances, the pharmaceutically effective amount of tolebrutinib is at a dose of about 50 mg to about 130 mg. In some instances, the pharmaceutically effective amount of tolebrutinib is at a dose of 60 mg. In some instances, the pharmaceutically effective amount of tolebrutinib is at a dose of 120 mg. In some instances, the pharmaceutically effective amount of tolebrutinib is administered orally. In some instances, the pharmaceutically effective amount of tolebrutinib is administered daily. In an embodiment, the method of treating a subject having MS further comprises administering a pharmaceutically effective amount of an anti-CD20 antibody. In some instances, the anti-CD20 antibody dose comprises one or more intravenous infusion (e.g., intravenous (IV) infusion) of an anti-CD20 antibody. The one or more IV infusions can be administered over a period of time (e.g., for at least six months). In some instances the subject is administered the IV infusion at doses that are about a week to about three weeks (e.g., about 1 week, about 2 weeks, or about 3 weeks) apart. In some instances, the anti-CD20 antibody is administered during treatment with tolebrutinib. In some instances, the anti-CD20 antibody is administered separately from treatment with tolebrutinib. In some instances, the anti-CD20 antibody is administered before treatment with tolebrutinib. In some instances, the anti-CD20 antibody is administered after treatment with tolebrutinib.

In another embodiment, disclosed herein is a method of identifying a patient as being suitable for participating in a clinical trial for MS, the method comprising: (a) detecting CD27 in a biological sample comprising CSF from the subject; and (b) identifying the subject expressing CD27 in the biological sample, thereby identifying a patient as being suitable for participating in a clinical trial for MS.

In yet another embodiment, disclosed herein is a method of diagnosing a subject as having MS, the method comprising: (a) detecting CD27 in a biological sample comprising CSF from the subject; and (b) identifying the subject expressing CD27 in the biological sample as having MS.

In yet another embodiment, disclosed herein is a method of identifying a subject having MS as expressing CD27 in a biological sample comprising CSF, the method comprising: (a) detecting CD27 in the biological sample; and (b) identifying the subject having MS expressing CD27 in the biological sample.

Also disclosed herein is a method of identifying a subject as having an increased likelihood of developing MS, the method comprising: (a) detecting CD27 in a biological sample comprising CSF from a subject; and (b) identifying a subject expressing CD27 in the biological sample, as having an increased likelihood of developing MS.

In another embodiment, disclosed herein is a method of identifying a subject as likely to develop MS, the method comprising: (a) detecting CD27 in a biological sample comprising CSF from a subject; and (b) identifying a subject expressing CD27 in the biological sample, as having an increased likelihood of developing MS.

In some instances, the CD27 is increased compared to CD27 in a reference sample. In some instances, the reference sample is from a second subject. In some instances, the second subject does not have MS. In some instances, the reference sample comprises CSF. In some instances, the method further includes obtaining the biological sample from the subject.

In some instances, the disclosure also provides a method of monitoring progression of MS in a subject over time, the method comprising: (a) detecting CD27 in a first biological sample obtained from a subject at a first time point; (b) detecting CD27 in a second biological sample obtained from the subject at a second time point; and (c) identifying: (i) a subject having increased CD27 at the second time point, as compared to CD27 at the first time point, as having progressing MS, or (ii) a subject having about the same or a decreased CD27 at the second time point, as compared to CD27 at the first time point, as having static or regressing MS.

In some instances, the method includes administering a pharmaceutically effective amount of tolebrutinib to the subject. In some instances, the pharmaceutically effective amount of tolebrutinib is at a dose of about 50 mg to about 130 mg. In some instances, the pharmaceutically effective amount of tolebrutinib is at a dose of 60 mg. In some instances, the pharmaceutically effective amount of tolebrutinib is at a dose of 120 mg. In some instances, the pharmaceutically effective amount of tolebrutinib is administered orally. In some instances, the pharmaceutically effective amount of tolebrutinib is administered daily. In an embodiment, the method further comprises administering a pharmaceutically effective amount of an anti-CD20 antibody to the subject. In some instances, the anti-CD20 antibody dose comprises one or more intravenous infusion (e.g., intravenous (IV) infusion) of an anti-CD20 antibody. The one or more IV infusions can be administered over a period of time (e.g., for at least six months). In some instances the subject is administered the IV infusion at doses that are about a week to about three weeks (e.g., about 1 week, about 2 weeks, or about 3 weeks) apart. In some instances, the anti-CD20 antibody is administered during treatment with tolebrutinib. In some instances, the anti-CD20 antibody is administered separately from treatment with tolebrutinib. In some instances the anti-CD20 antibody is administered before treatment with tolebrutinib. In some instances the anti-CD20 antibody is administered after treatment with tolebrutinib.

In some instances, the methods disclosed herein include a method of assessing the efficacy of a treatment of a pharmaceutically effective amount of tolebrutinib in a subject having MS, the method comprising: (a) detecting (i) CD27 in a first biological sample comprising CSF obtained from the subject at a first time point and (ii) CD27 in a second biological sample comprising CSF obtained from the subject at a second time point, wherein the subject is administered one or more doses of the treatment between the first and second time points; and (b) determining a correlation between efficacy of the treatment and CD27 in the second biological sample as compared to CD27 in a sample obtained from an untreated patient, wherein the CD27 in the second biological sample is about the same or decreased as compared to the abundance in the sample from the untreated patient, thereby indicating that the treatment is effective for MS in the subject. In an embodiment, the method further comprises administering a pharmaceutically effective amount of an anti-CD20 antibody to the subject and assessing the efficacy of a treatment of a pharmaceutically effective amount of tolebrutinib and anti-CD20 antibody.

In some instances, the difference between the first time point and the second time point is about 1 month to about two years. In some instances, the pharmaceutically effective amount of tolebrutinib is administered at a dose of about 50 mg to about 130 mg. In some instances, the pharmaceutically effective amount of tolebrutinib is at a dose of 60 mg. In some instances, the pharmaceutically effective amount of tolebrutinib is at a dose of 120 mg. In some instances, the pharmaceutically effective amount of tolebrutinib is administered orally. In some instances, the pharmaceutically effective amount of tolebrutinib is administered daily. In some instances, the anti-CD20 antibody dose comprises one or more intravenous infusion (e.g., intravenous (IV) infusion) of an anti-CD20 antibody. The one or more IV infusions can be administered over a period of time (e.g., for at least six months). In some instances, the subject is administered the IV infusion at doses that are about a week to about three weeks (e.g., about 1 week, about 2 weeks, or about 3 weeks) apart. In some instances, the CD27 comprises CD27 RNA. In some instances, the CD27 RNA is determined by polymerase chain reaction, quantitative polymerase chain reaction, or Northern blot. In some instances, the CD27 comprises CD27 protein. In some instances, the CD27 is determined by flow cytometry or Western blot. In some instances, the method further comprises monitoring the subject for the development of symptoms of MS. In some instances, the method further comprises administering to the subject a treatment for decreasing the rate of progression or decreasing the likelihood or susceptibility of developing MS.

In some instances, the preceding methods include administering additional or increased doses of the pharmaceutically effective amount of tolebrutinib for the subject.

In some instances, the subject has received a treatment for MS prior to detecting CD27. In some instances, the subject has been treated previously with an anti-CD20 therapy.

In some instances, the anti-CD20 therapy is ocrelizumab or rituximab.

In some instances, the subject comprises one or more brain lesions.

In some instances, the subject is administered one or more doses of an anti-CD20 therapy and/or a booster of an anti-CD20 therapy in addition to the pharmaceutically effective amount of tolebrutinib.

In some instances, administering of the pharmaceutically effective amount of tolebrutinib (and potentially with one or more additional therapies, such as an anti-CD20 therapy) results in one or more of: reduced brain lesions; a reduced amount of iron in brain tissue of the subject; and/or a decrease in synaptic density. In some instances, reduced or decrease(d) is compared to a previous amount from the same subject.

In some instances, the MS is relapsing-remitting multiple sclerosis. In some instances, the MS is secondary progressive multiple sclerosis.

The present disclosure relates to detection of NEFL as a biomarker to predict or confirm a treatment response for MS. The present disclosure has also identified that certain treatments can result in changes of NEFL biomarker expression. In particular, one embodiment disclosed herein includes a treatment change (e.g., from ocrelizumab (Ocrevus®) to tolebrutinib). The present disclosure has identified that NEFL can be used as a biomarker of prognosis, development, and therapeutic efficacy in the cerebrospinal fluid (CSF) of a subject with MS. Modulation of NEFL (e.g., a decrease in NEFL) protein expression is associated with more favorable outcomes of MS. Thus, provided herein are methods of detecting NEFL (both RNA and protein) as a biomarker that indicates severity of MS, including the progression, regression, or static presence of lesions (e.g., active lesions) in the brain of a subject with MS.

Thus, in one aspect, provided herein a method of treating a subject having MS, the method comprising: (a) detecting NEFL in cerebrospinal fluid (CSF) in the subject; and (b) administering a pharmaceutically effective amount of tolebrutinib to the subject. In a second aspect, disclosed herein is a method of treating a subject having MS, the method comprising: (a) detecting NEFL in a biological sample comprising CSF from the subject; (b) identifying the subject expressing NEFL in the biological sample as having MS; and (c) administering a pharmaceutically effective amount of tolebrutinib to the subject. In some instances, the pharmaceutically effective amount of tolebrutinib is at a dose of about 50 mg to about 130 mg. In some instances, the pharmaceutically effective amount of tolebrutinib is at a dose of 60 mg. In some instances, the pharmaceutically effective amount of tolebrutinib is at a dose of 120 mg. In some instances, the pharmaceutically effective amount of tolebrutinib is administered orally. In some instances, the pharmaceutically effective amount of tolebrutinib is administered daily. In an embodiment, the method of treating a subject having MS further comprises administering a pharmaceutically effective amount of an anti-CD20 antibody. In some instances, the anti-CD20 antibody dose comprises one or more intravenous infusion (e.g., intravenous (IV) infusion) of an anti-CD20 antibody. The one or more IV infusions can be administered over a period of time (e.g., for at least six months). In some instances, the subject is administered the IV infusion at doses that are about a week to about three weeks (e.g., about 1 week, about 2 weeks, or about 3 weeks) apart. In some instances, the anti-CD20 antibody is administered during treatment with tolebrutinib. In some instances, the anti-CD20 antibody is administered separately from treatment with tolebrutinib. In some instances, the anti-CD20 antibody is administered before treatment with tolebrutinib. In some instances, the anti-CD20 antibody is administered after treatment with tolebrutinib.

In another embodiment, disclosed herein is a method of identifying a patient as being suitable for participating in a clinical trial for MS, the method comprising: (a) detecting NEFL in a biological sample comprising CSF from the subject; and (b) identifying the subject expressing NEFL in the biological sample, thereby identifying a patient as being suitable for participating in a clinical trial for MS.

In yet another embodiment, disclosed herein is a method of diagnosing a subject as having MS, the method comprising: (a) detecting NEFL in a biological sample comprising CSF from the subject; and (b) identifying the subject expressing NEFL in the biological sample as having MS.

In yet another embodiment, disclosed herein is a method of identifying a subject having MS as expressing NEFL in a biological sample comprising CSF, the method comprising: (a) detecting NEFL in the biological sample; and (b) identifying the subject having MS expressing NEFL in the biological sample.

Also disclosed herein is a method of identifying a subject as having an increased likelihood of developing MS, the method comprising: (a) detecting NEFL in a biological sample comprising CSF from a subject; and (b) identifying a subject expressing NEFL in the biological sample, as having an increased likelihood of developing MS.

In another embodiment, disclosed herein is a method of identifying a subject as likely to develop MS, the method comprising: (a) detecting NEFL in a biological sample comprising CSF from a subject; and (b) identifying a subject expressing NEFL in the biological sample, as having an increased likelihood of developing MS.

In some instances, the NEFL is increased compared to NEFL in a reference sample. In some instances, the reference sample is from a second subject. In some instances, the second subject does not have MS. In some instances, the reference sample comprises CSF. In some instances, the method further includes obtaining the biological sample from the subject.

In some instances, the disclosure also provides a method of monitoring progression of MS in a subject over time, the method comprising: (a) detecting NEFL in a first biological sample obtained from a subject at a first time point; (b) detecting NEFL in a second biological sample obtained from the subject at a second time point; and (c) identifying: (i) a subject having increased NEFL at the second time point, as compared to NEFL at the first time point, as having progressing MS, or (ii) a subject having about the same or a decreased NEFL at the second time point, as compared to NEFL at the first time point, as having static or regressing MS.

In some instances, the method includes administering a pharmaceutically effective amount of tolebrutinib to the subject. In some instances, the pharmaceutically effective amount of tolebrutinib is at a dose of about 50 mg to about 130 mg. In some instances, the pharmaceutically effective amount of tolebrutinib is at a dose of 60 mg. In some instances, the pharmaceutically effective amount of tolebrutinib is at a dose of 120 mg. In some instances, the pharmaceutically effective amount of tolebrutinib is administered orally. In some instances, the pharmaceutically effective amount of tolebrutinib is administered daily. In an embodiment, the method further comprises administering a pharmaceutically effective amount of an anti-CD20 antibody to the subject. In some instances, the anti-CD20 antibody dose comprises one or more intravenous infusion (e.g., intravenous (IV) infusion) of an anti-CD20 antibody. The one or more IV infusions can be administered over a period of time (e.g., for at least six months). In some instances the subject is administered the IV infusion at doses that are about a week to about three weeks (e.g., about 1 week, about 2 weeks, or about 3 weeks) apart. In some instances, the anti-CD20 antibody is administered concurrently with tolebrutinib. In some instances, the anti-CD20 antibody is administered separately from tolebrutinib. In some instances, the anti-CD20 antibody is administered before tolebrutinib. In some instances, the anti-CD20 antibody is administered after tolebrutinib.

In some instances, the methods disclosed herein include a method of assessing the efficacy of a treatment of a pharmaceutically effective amount of tolebrutinib in a subject having MS, the method comprising: (a) detecting (i) NEFL in a first biological sample comprising CSF obtained from the subject at a first time point and (ii) NEFL in a second biological sample comprising CSF obtained from the subject at a second time point, wherein the subject is administered one or more doses of the treatment between the first and second time points; and (b) determining a correlation between efficacy of the treatment and NEFL in the second biological sample as compared to NEFL in a sample obtained from an untreated patient, wherein the NEFL in the second biological sample is about the same or decreased as compared to the abundance in the sample from the untreated patient, thereby indicating that the treatment is effective for MS in the subject. In an embodiment, the method further comprises administering a pharmaceutically effective amount of an anti-CD20 antibody to the subject and assessing the efficacy of a treatment of a pharmaceutically effective amount of tolebrutinib and anti-CD20 antibody.

In some instances, the difference between the first time point and the second time point is about 1 month to about two years. In some instances, the pharmaceutically effective amount of tolebrutinib is administered at a dose of about 50 mg to about 130 mg. In some instances, the pharmaceutically effective amount of tolebrutinib is at a dose of 60 mg. In some instances, the pharmaceutically effective amount of tolebrutinib is at a dose of 120 mg. In some instances, the pharmaceutically effective amount of tolebrutinib is administered orally. In some instances, the pharmaceutically effective amount of tolebrutinib is administered daily. In some instances, the anti-CD20 antibody dose comprises one or more intravenous infusion (e.g., intravenous (IV) infusion) of an anti-CD20 antibody. The one or more IV infusions can be administered over a period of time (e.g., for at least six months). In some instances, the subject is administered the IV infusion at doses that are about a week to about three weeks (e.g., about 1 week, about 2 weeks, or about 3 weeks) apart. In some instances, the NEFL comprises NEFL RNA. In some instances, the NEFL RNA is determined by polymerase chain reaction, quantitative polymerase chain reaction, or Northern blot. In some instances, the NEFL comprises NEFL protein. In some instances, the NEFL is determined by flow cytometry or Western blot. In some instances, the method further comprises monitoring the subject for the development of symptoms of MS. In some instances, the method further comprises administering to the subject a treatment for decreasing the rate of progression or decreasing the likelihood or susceptibility of developing MS.

In some instances, the preceding methods include administering additional or increased doses of the pharmaceutically effective amount of tolebrutinib for the subject. In some instances, the preceding methods include administering additional or increased doses of the pharmaceutically effective amount of tolebrutinib and an anti-CD20 antibody for the subject.

In some instances, the subject has received a treatment for MS prior to detecting NEFL. In some instances, the subject has been treated previously with an anti-CD20 therapy.

In some instances, the anti-CD20 therapy is ocrelizumab or rituximab.

In some instances, the subject comprises one or more brain lesions.

In some instances, the subject is administered one or more doses of an anti-CD20 therapy and/or one or more doses of a booster of an anti-CD20 therapy in addition to the pharmaceutically effective amount of tolebrutinib.

In some instances, administering of the pharmaceutically effective amount of tolebrutinib (and potentially with one or more additional therapies, such as an anti-CD20 therapy) results in one or more of: reduced brain lesions; a reduced amount of iron in brain tissue of the subject; and/or a decrease in synaptic density. In some instances, reduced or decrease(d) is compared to a previous amount from the same subject.

In some instances, the MS is relapsing-remitting multiple sclerosis. In some instances, the MS is secondary progressive multiple sclerosis.

The present disclosure relates to detection of CCL4 as a biomarker to predict or confirm a treatment response for MS. The present disclosure has also identified that certain treatments can result in changes of CCL4 biomarker expression. In particular, one embodiment disclosed herein includes a treatment change (e.g., from ocrelizumab (Ocrevus®) to tolebrutinib). The present disclosure has identified that CCL4 can be used as a biomarker of prognosis, development, and therapeutic efficacy in the cerebrospinal fluid (CSF) of a subject with MS. Modulation of CCL4 (e.g., a decrease in CCL4) protein expression is associated with more favorable outcomes of MS. Thus, provided herein are methods of detecting CCL4 (both RNA and protein) as a biomarker that indicates severity of MS, including the progression, regression, or static presence of lesions (e.g., active lesions) in the brain of a subject with MS.

Thus, in one aspect, provided herein a method of treating a subject having MS, the method comprising: (a) detecting CCL4 in cerebrospinal fluid (CSF) in the subject; and (b) administering a pharmaceutically effective amount of tolebrutinib to the subject. In a second aspect, disclosed herein is a method of treating a subject having MS, the method comprising: (a) detecting CCL4 in a biological sample comprising CSF from the subject; (b) identifying the subject expressing CCL4 in the biological sample as having MS; and (c) administering a pharmaceutically effective amount of tolebrutinib to the subject. In some instances, the pharmaceutically effective amount of tolebrutinib is at a dose of about 50 mg to about 130 mg. In some instances, the pharmaceutically effective amount of tolebrutinib is at a dose of 60 mg. In some instances, the pharmaceutically effective amount of tolebrutinib is at a dose of 120 mg. In some instances, the pharmaceutically effective amount of tolebrutinib is administered orally. In some instances, the pharmaceutically effective amount of tolebrutinib is administered daily. In an embodiment, the method of treating a subject having MS further comprises administering a pharmaceutically effective amount of an anti-CD20 antibody. In some instances, the anti-CD20 antibody comprises ocrelizumab or rituximab. In some instances, the anti-CD20 antibody dose comprises one or more intravenous infusion (e.g., intravenous (IV) infusion) of an anti-CD20 antibody. The one or more IV infusions can be administered over a period of time (e.g., for at least six months). In some instances the subject is administered the IV infusion at doses that are about a week to about three weeks (e.g., about 1 week, about 2 weeks, or about 3 weeks) apart. In some instances, the anti-CD20 antibody is administered during treatment with tolebrutinib. In some instances, the anti-CD20 antibody is administered separately from treatment with tolebrutinib. In some instances the anti-CD20 antibody is administered before treatment with tolebrutinib. In some instances the anti-CD20 antibody is administered after treatment with tolebrutinib.

In another embodiment, disclosed herein is a method of identifying a patient as being suitable for participating in a clinical trial for MS, the method comprising: (a) detecting CCL4 in a biological sample comprising CSF from the subject; and (b) identifying the subject expressing CCL4 in the biological sample, thereby identifying a patient as being suitable for participating in a clinical trial for MS.

In yet another embodiment, disclosed herein is a method of diagnosing a subject as having MS, the method comprising: (a) detecting CCL4 in a biological sample comprising CSF from the subject; and (b) identifying the subject expressing CCL4 in the biological sample as having MS.

In yet another embodiment, disclosed herein is a method of identifying a subject having MS as expressing CCL4 in a biological sample comprising CSF, the method comprising: (a) detecting CCL4 in the biological sample; and (b) identifying the subject having MS expressing CCL4 in the biological sample.

Also disclosed herein is a method of identifying a subject as having an increased likelihood of developing MS, the method comprising: (a) detecting CCL4 in a biological sample comprising CSF from a subject; and (b) identifying a subject expressing CCL4 in the biological sample, as having an increased likelihood of developing MS.

In another embodiment, disclosed herein is a method of identifying a subject as likely to develop MS, the method comprising: (a) detecting CCL4 in a biological sample comprising CSF from a subject; and (b) identifying a subject expressing CCL4 in the biological sample, as having an increased likelihood of developing MS.

In some instances, the CCL4 is increased compared to CCL4 in a reference sample. In some instances, the reference sample is from a second subject. In some instances, the second subject does not have MS. In some instances, the reference sample comprises CSF. In some instances, the method further includes obtaining the biological sample from the subject.

In some instances, the disclosure also provides a method of monitoring progression of MS in a subject over time, the method comprising: (a) detecting CCL4 in a first biological sample obtained from a subject at a first time point; (b) detecting CCL4 in a second biological sample obtained from the subject at a second time point; and (c) identifying: (i) a subject having increased CCL4 at the second time point, as compared to CCL4 at the first time point, as having progressing MS, or (ii) a subject having about the same or a decreased CCL4 at the second time point, as compared to CCL4 at the first time point, as having static or regressing MS.

In some instances, the method includes administering a pharmaceutically effective amount of tolebrutinib to the subject. In some instances, the pharmaceutically effective amount of tolebrutinib is at a dose of about 50 mg to about 130 mg. In some instances, the pharmaceutically effective amount of tolebrutinib is at a dose of 60 mg. In some instances, the pharmaceutically effective amount of tolebrutinib is at a dose of 120 mg. In some instances, the pharmaceutically effective amount of tolebrutinib is administered orally. In some instances, the pharmaceutically effective amount of tolebrutinib is administered daily. In an embodiment, the method further comprises administering a pharmaceutically effective amount of an anti-CD20 antibody to the subject. In some instances, the anti-CD20 antibody dose comprises one or more intravenous infusion (e.g., intravenous (IV) infusion) of an anti-CD20 antibody. The one or more IV infusions can be administered over a period of time (e.g., for at least six months). In some instances, the subject is administered the IV infusion at doses that are about a week to about three weeks (e.g., about 1 week, about 2 weeks, or about 3 weeks) apart. In some instances, the anti-CD20 antibody is administered during treatment with tolebrutinib. In some instances, the anti-CD20 antibody is administered separately from treatment with tolebrutinib. In some instances, the anti-CD20 antibody is administered before treatment with tolebrutinib. In some instances, the anti-CD20 antibody is administered after treatment with tolebrutinib.

In some instances, the methods disclosed herein include a method of assessing the efficacy of a treatment of a pharmaceutically effective amount of tolebrutinib in a subject having MS, the method comprising: (a) detecting (i) CCL4 in a first biological sample comprising CSF obtained from the subject at a first time point and (ii) CCL4 in a second biological sample comprising CSF obtained from the subject at a second time point, wherein the subject is administered one or more doses of the treatment between the first and second time points; and (b) determining a correlation between efficacy of the treatment and CCL4 in the second biological sample as compared to CCL4 in a sample obtained from an untreated patient, wherein the CCL4 in the second biological sample is about the same or decreased as compared to the abundance in the sample from the untreated patient, thereby indicating that the treatment is effective for MS in the subject. In an embodiment, the method further comprises administering a pharmaceutically effective amount of an anti-CD20 antibody to the subject and assessing the efficacy of a treatment of a pharmaceutically effective amount of tolebrutinib and anti-CD20 antibody.

In some instances, the difference between the first time point and the second time point is about 1 month to about two years. In some instances, the pharmaceutically effective amount of tolebrutinib is administered at a dose of about 50 mg to about 130 mg. In some instances, the pharmaceutically effective amount of tolebrutinib is at a dose of 60 mg. In some instances, the pharmaceutically effective amount of tolebrutinib is at a dose of 120 mg. In some instances, the pharmaceutically effective amount of tolebrutinib is administered orally. In some instances, the pharmaceutically effective amount of tolebrutinib is administered daily. In some instances, the anti-CD20 antibody dose comprises one or more intravenous infusion (e.g., intravenous (IV) infusion) of an anti-CD20 antibody. The one or more IV infusions can be administered over a period of time (e.g., for at least six months). In some instances, the subject is administered the IV infusion at doses that are about a week to about three weeks (e.g., about 1 week, about 2 weeks, or about 3 weeks) apart. In some instances, the CCL4 comprises CCL4 RNA. In some instances, the CCL4 RNA is determined by polymerase chain reaction, quantitative polymerase chain reaction, or Northern blot. In some instances, the CCL4 comprises CCL4 protein. In some instances, the CCL4 is determined by flow cytometry or Western blot. In some instances, the method further comprises monitoring the subject for the development of symptoms of MS. In some instances, the method further comprises administering to the subject a treatment for decreasing the rate of progression or decreasing the likelihood or susceptibility of developing MS.

In some instances, the preceding methods include administering additional or increased doses of the pharmaceutically effective amount of tolebrutinib for the subject. In some instances, the preceding methods include administering additional or increased doses of the pharmaceutically effective amount of tolebrutinib and an anti-CD20 antibody for the subject.

In some instances, the subject has received a treatment for MS prior to detecting CCL4. In some instances, the subject has been treated previously with an anti-CD20 therapy.

In some instances, the anti-CD20 therapy is ocrelizumab or rituximab.

In some instances, the subject comprises one or more brain lesions.

In some instances, the subject is administered one or more doses of an anti-CD20 therapy and/or one or more doses of booster of an anti-CD20 therapy in addition to the pharmaceutically effective amount of tolebrutinib.

In some instances, administering of the pharmaceutically effective amount of tolebrutinib (and potentially with one or more additional therapies, such as an anti-CD20 therapy) results in one or more of: reduced brain lesions; a reduced amount of iron in brain tissue of the subject; and/or a decrease in synaptic density. In some instances, reduced or decrease(d) is compared to a previous amount from the same subject.

In some instances, the MS is relapsing-remitting multiple sclerosis. In some instances, the MS is secondary progressive multiple sclerosis.

The present disclosure relates to detection of CCL3 as a biomarker to predict or confirm a treatment response for MS. The present disclosure has also identified that certain treatments can result in changes of CCL3 biomarker expression. In particular, one embodiment disclosed herein includes a treatment change (e.g., from ocrelizumab (Ocrevus®) to tolebrutinib). The present disclosure has identified that CCL3 can be used as a biomarker of prognosis, development, and therapeutic efficacy in the cerebrospinal fluid (CSF) of a subject with MS. Modulation of CCL3 (e.g., a decrease in CCL3) protein expression is associated with more favorable outcomes of MS. Thus, provided herein are methods of detecting CCL3 (both RNA and protein) as a biomarker that indicates severity of MS, including the progression, regression, or static presence of lesions (e.g., active lesions) in the brain of a subject with MS.

Thus, in one aspect, provided herein a method of treating a subject having MS, the method comprising: (a) detecting CCL3 in cerebrospinal fluid (CSF) in the subject; and (b) administering a pharmaceutically effective amount of tolebrutinib to the subject. In a second aspect, disclosed herein is a method of treating a subject having MS, the method comprising: (a) detecting CCL3 in a biological sample comprising CSF from the subject; (b) identifying the subject expressing CCL3 in the biological sample as having MS; and (c) administering a pharmaceutically effective amount of tolebrutinib to the subject. In some instances, the pharmaceutically effective amount of tolebrutinib is at a dose of about 50 mg to about 130 mg. In some instances, the pharmaceutically effective amount of tolebrutinib is at a dose of 60 mg. In some instances, the pharmaceutically effective amount of tolebrutinib is at a dose of 120 mg. In some instances, the pharmaceutically effective amount of tolebrutinib is administered orally. In some instances, the pharmaceutically effective amount of tolebrutinib is administered daily. In some instances, the pharmaceutically effective amount of tolebrutinib is administered daily. In an embodiment, the method of treating a subject having MS further comprises administering a pharmaceutically effective amount of an anti-CD20 antibody. In some instances, the anti-CD20 antibody comprises ocrelizumab or rituximab. In some instances, the anti-CD20 antibody dose comprises one or more intravenous infusion (e.g., intravenous (IV) infusion) of an anti-CD20 antibody. The one or more IV infusions can be administered over a period of time (e.g., for at least six months). In some instances, the subject is administered the IV infusion at doses that are about a week to about three weeks (e.g., about 1 week, about 2 weeks, or about 3 weeks) apart. In some instances, the anti-CD20 antibody is administered during treatment with tolebrutinib. In some instances, the anti-CD20 antibody is administered separately from treatment with tolebrutinib. In some instances, the anti-CD20 antibody is administered before treatment with tolebrutinib. In some instances, the anti-CD20 antibody is administered after treatment with tolebrutinib.

In another embodiment, disclosed herein is a method of identifying a patient as being suitable for participating in a clinical trial for MS, the method comprising: (a) detecting CCL3 in a biological sample comprising CSF from the subject; and (b) identifying the subject expressing CCL3 in the biological sample, thereby identifying a patient as being suitable for participating in a clinical trial for MS.

In yet another embodiment, disclosed herein is a method of diagnosing a subject as having MS, the method comprising: (a) detecting CCL3 in a biological sample comprising CSF from the subject; and (b) identifying the subject expressing CCL3 in the biological sample as having MS.

In yet another embodiment, disclosed herein is a method of identifying a subject having MS as expressing CCL3 in a biological sample comprising CSF, the method comprising: (a) detecting CCL3 in the biological sample; and (b) identifying the subject having MS expressing CCL3 in the biological sample.

Also disclosed herein is a method of identifying a subject as having an increased likelihood of developing MS, the method comprising: (a) detecting CCL3 in a biological sample comprising CSF from a subject; and (b) identifying a subject expressing CCL3 in the biological sample, as having an increased likelihood of developing MS.

In another embodiment, disclosed herein is a method of identifying a subject as likely to develop MS, the method comprising: (a) detecting CCL3 in a biological sample comprising CSF from a subject; and (b) identifying a subject expressing CCL3 in the biological sample, as having an increased likelihood of developing MS.

In some instances, the CCL3 is increased compared to CCL3 in a reference sample. In some instances, the reference sample is from a second subject. In some instances, the second subject does not have MS. In some instances, the reference sample comprises CSF. In some instances, the method further includes obtaining the biological sample from the subject.

In some instances, the disclosure also provides a method of monitoring progression of MS in a subject over time, the method comprising: (a) detecting CCL3 in a first biological sample obtained from a subject at a first time point; (b) detecting CCL3 in a second biological sample obtained from the subject at a second time point; and (c) identifying: (i) a subject having increased CCL3 at the second time point, as compared to CCL3 at the first time point, as having progressing MS, or (ii) a subject having about the same or a decreased CCL3 at the second time point, as compared to CCL3 at the first time point, as having static or regressing MS.

In some instances, the method includes administering a pharmaceutically effective amount of tolebrutinib to the subject. In some instances, the pharmaceutically effective amount of tolebrutinib is at a dose of about 50 mg to about 130 mg. In some instances, the pharmaceutically effective amount of tolebrutinib is at a dose of 60 mg. In some instances, the pharmaceutically effective amount of tolebrutinib is at a dose of 120 mg. In some instances, the pharmaceutically effective amount of tolebrutinib is administered orally. In some instances, the pharmaceutically effective amount of tolebrutinib is administered daily. In an embodiment, the method further comprises administering a pharmaceutically effective amount of an anti-CD20 antibody to the subject. In some instances, the anti-CD20 antibody dose comprises one or more intravenous infusion (e.g., intravenous (IV) infusion) of an anti-CD20 antibody. The one or more IV infusions can be administered over a period of time (e.g., for at least six months). In some instances the subject is administered the IV infusion at doses that are about a week to about three weeks (e.g., about 1 week, about 2 weeks, or about 3 weeks) apart. In some instances, the anti-CD20 antibody is administered during treatment with tolebrutinib. In some instances, the anti-CD20 antibody is administered separately from treatment with tolebrutinib. In some instances, the anti-CD20 antibody is administered before treatment with tolebrutinib. In some instances, the anti-CD20 antibody is administered after treatment with tolebrutinib.

In some instances, the methods disclosed herein include a method of assessing the efficacy of a treatment of a pharmaceutically effective amount of tolebrutinib in a subject having MS, the method comprising: (a) detecting (i) CCL3 in a first biological sample comprising CSF obtained from the subject at a first time point and (ii) CCL3 in a second biological sample comprising CSF obtained from the subject at a second time point, wherein the subject is administered one or more doses of the treatment between the first and second time points; and (b) determining a correlation between efficacy of the treatment and CCL3 in the second biological sample as compared to CCL3 in a sample obtained from an untreated patient, wherein the CCL3 in the second biological sample is about the same or decreased as compared to the abundance in the sample from the untreated patient, thereby indicating that the treatment is effective for MS in the subject. In an embodiment, the method further comprises administering a pharmaceutically effective amount of an anti-CD20 antibody to the subject and assessing the efficacy of a treatment of a pharmaceutically effective amount of tolebrutinib and anti-CD20 antibody.

In some instances, the difference between the first time point and the second time point is about 1 month to about two years. In some instances, the pharmaceutically effective amount of tolebrutinib is administered at a dose of about 50 mg to about 130 mg. In some instances, the pharmaceutically effective amount of tolebrutinib is at a dose of 60 mg. In some instances, the pharmaceutically effective amount of tolebrutinib is at a dose of 120 mg. In some instances, the pharmaceutically effective amount of tolebrutinib is administered orally. In some instances, the pharmaceutically effective amount of tolebrutinib is administered daily. In some instances, the anti-CD20 antibody dose comprises one or more intravenous infusion (e.g., intravenous (IV) infusion) of an anti-CD20 antibody. The one or more IV infusions can be administered over a period of time (e.g., for at least six months). In some instances, the subject is administered the IV infusion at doses that are about a week to about three weeks (e.g., about 1 week, about 2 weeks, or about 3 weeks) apart. In some instances, the CCL3 comprises CCL3 RNA. In some instances, the CCL3 RNA is determined by polymerase chain reaction, quantitative polymerase chain reaction, or Northern blot. In some instances, the CCL3 comprises CCL3 protein. In some instances, the CCL3 is determined by flow cytometry or Western blot. In some instances, the method further comprises monitoring the subject for the development of symptoms of MS. In some instances, the method further comprises administering to the subject a treatment for decreasing the rate of progression or decreasing the likelihood or susceptibility of developing MS.

In some instances, the preceding methods include administering additional or increased doses of the pharmaceutically effective amount of tolebrutinib for the subject. In some instances, the preceding methods include administering additional or increased doses of the pharmaceutically effective amount of tolebrutinib and an anti-CD20 antibody for the subject.

In some instances, the subject has received a treatment for MS prior to detecting CCL3. In some instances, the subject has been treated previously with an anti-CD20 therapy.

In some instances, the anti-CD20 therapy is ocrelizumab or rituximab.

In some instances, the subject comprises one or more brain lesions.

In some instances, the subject is administered one or more doses of an anti-CD20 therapy and/or a booster of an anti-CD20 therapy in addition to the pharmaceutically effective amount of tolebrutinib.

In some instances, administering of the pharmaceutically effective amount of tolebrutinib (and potentially with one or more additional therapies, such as anti-CD20 therapy) results in one or more of: reduced brain lesions; a reduced amount of iron in brain tissue of the subject; and/or a decrease in synaptic density. In some instances, reduced or decrease(d) is compared to a previous amount from the same subject.

In some instances, the MS is relapsing-remitting multiple sclerosis. In some instances, the MS is secondary progressive multiple sclerosis.

The present disclosure also relates to detection of CXCL10, CXCL13, CD27, NEFL, CCL4, and/or CCL3 as a biomarker to predict or confirm a treatment response for MS. The present disclosure has also identified that certain treatments can result in changes of CXCL10, CXCL13, CD27, NEFL, CCL4, and/or CCL3 biomarker expression. In particular, one embodiment disclosed herein includes a treatment change (e.g., from ocrelizumab (Ocrevus®) to tolebrutinib). The present disclosure has identified that CXCL10, CXCL13, CD27, NEFL, CCL4, and/or CCL3 can be used as a biomarker of prognosis, development, and therapeutic efficacy in the cerebrospinal fluid (CSF) of a subject with MS. Modulation of CXCL10, CXCL13, CD27, NEFL, CCL4, and/or CCL3 (e.g., a decrease in CXCL10, CXCL13, CD27, NEFL, CCL4, and/or CCL3) protein expression is associated with more favorable outcomes of MS. Thus, provided herein are methods of detecting CXCL10, CXCL13, CD27, NEFL, CCL4, and/or CCL3 (both RNA and protein) as a biomarker that indicates severity of MS, including the progression, regression, or static presence of lesions (e.g., active lesions) in the brain of a subject with MS.

Thus, in one aspect, provided herein a method of treating a subject having MS, the method comprising: (a) detecting CXCL10, CXCL13, CD27, NEFL, CCL4, and/or CCL3 in cerebrospinal fluid (CSF) in the subject; and (b) administering a pharmaceutically effective amount of tolebrutinib to the subject. In a second aspect, disclosed herein is a method of treating a subject having MS, the method comprising: (a) detecting CXCL10, CXCL13, CD27, NEFL, CCL4, and/or CCL3 in a biological sample comprising CSF from the subject; (b) identifying the subject expressing CXCL10, CXCL13, CD27, NEFL, CCL4, and/or CCL3 in the biological sample as having MS; and (c) administering a pharmaceutically effective amount of tolebrutinib to the subject. In some instances, the pharmaceutically effective amount of tolebrutinib is at a dose of about 50 mg to about 130 mg. In some instances, the pharmaceutically effective amount of tolebrutinib is at a dose of 60 mg. In some instances, the pharmaceutically effective amount of tolebrutinib is at a dose of 120 mg. In some instances, the pharmaceutically effective amount of tolebrutinib is administered orally. In some instances, the pharmaceutically effective amount of tolebrutinib is administered daily.

In an embodiment, the method of treating a subject having MS further comprises administering a pharmaceutically effective amount of an anti-CD20 antibody. In some instances, the anti-CD20 antibody comprises ocrelizumab or rituximab. In some instances, the anti-CD20 antibody dose comprises one or more intravenous infusion (e.g., intravenous (IV) infusion) of an anti-CD20 antibody. The one or more IV infusions can be administered over a period of time (e.g., for at least six months). In some instances the subject is administered the IV infusion at doses that are about a week to about three weeks (e.g., about 1 week, about 2 weeks, or about 3 weeks) apart. In some instances, the anti-CD20 antibody is administered during treatment with tolebrutinib. In some instances, the anti-CD20 antibody is administered separately from treatment with tolebrutinib. In some instances, the anti-CD20 antibody is administered before treatment with tolebrutinib. In some instances, the anti-CD20 antibody is administered after treatment with tolebrutinib.

In another embodiment, disclosed herein is a method of identifying a patient as being suitable for participating in a clinical trial for MS, the method comprising: (a) detecting CXCL10, CXCL13, CD27, NEFL, CCL4, and/or CCL3 in a biological sample comprising CSF from the subject; and (b) identifying the subject expressing CXCL10, CXCL13, CD27, NEFL, CCL4, and/or CCL3 in the biological sample, thereby identifying a patient as being suitable for participating in a clinical trial for MS.

In yet another embodiment, disclosed herein is a method of diagnosing a subject as having MS, the method comprising: (a) detecting CXCL10, CXCL13, CD27, NEFL, CCL4, and/or CCL3 in a biological sample comprising CSF from the subject; and (b) identifying the subject expressing CXCL10, CXCL13, CD27, NEFL, CCL4, and/or CCL3 in the biological sample as having MS.

In yet another embodiment, disclosed herein is a method of identifying a subject having MS as expressing CXCL10, CXCL13, CD27, NEFL, CCL4, and/or CCL3 in a biological sample comprising CSF, the method comprising: (a) detecting CXCL10, CXCL13, CD27, NEFL, CCL4, and/or CCL3 in the biological sample; and (b) identifying the subject having MS expressing CXCL10, CXCL13, CD27, NEFL, CCL4, and/or CCL3 in the biological sample.

Also disclosed herein is a method of identifying a subject as having an increased likelihood of developing MS, the method comprising: (a) detecting CXCL10, CXCL13, CD27, NEFL, CCL4, and/or CCL3 in a biological sample comprising CSF from a subject; and (b) identifying a subject expressing CXCL10, CXCL13, CD27, NEFL, CCL4, and/or CCL3 in the biological sample, as having an increased likelihood of developing MS.

In another embodiment, disclosed herein is a method of identifying a subject as likely to develop MS, the method comprising: (a) detecting CXCL10, CXCL13, CD27, NEFL, CCL4, and/or CCL3 in a biological sample comprising CSF from a subject; and (b) identifying a subject expressing CXCL10, CXCL13, CD27, NEFL, CCL4, and/or CCL3 in the biological sample, as having an increased likelihood of developing MS.

In some instances, the CXCL10, CXCL13, CD27, NEFL, CCL4, and/or CCL3 is increased compared to CXCL10, CXCL13, CD27, NEFL, CCL4, and/or CCL3 in a reference sample. In some instances, the reference sample is from a second subject. In some instances, the second subject does not have MS. In some instances, the reference sample comprises CSF. In some instances, the method further includes obtaining the biological sample from the subject.

In some instances, the disclosure also provides a method of monitoring progression of MS in a subject over time, the method comprising: (a) detecting CXCL10, CXCL13, CD27, NEFL, CCL4, and/or CCL3 in a first biological sample obtained from a subject at a first time point; (b) detecting CXCL10, CXCL13, CD27, NEFL, CCL4, and/or CCL3 in a second biological sample obtained from the subject at a second time point; and (c) identifying: (i) a subject having increased CXCL10, CXCL13, CD27, NEFL, CCL4, and/or CCL3 at the second time point, as compared to CXCL10, CXCL13, CD27, NEFL, CCL4, and/or CCL3 at the first time point, as having progressing MS, or (ii) a subject having about the same or a decreased CXCL10, CXCL13, CD27, NEFL, CCL4, and/or CCL3 at the second time point, as compared to CXCL10, CXCL13, CD27, NEFL, CCL4, and/or CCL3 at the first time point, as having static or regressing MS.

In some instances, the method includes administering a pharmaceutically effective amount of tolebrutinib to the subject. In some instances, the pharmaceutically effective amount of tolebrutinib is at a dose of about 50 mg to about 130 mg. In some instances, the pharmaceutically effective amount of tolebrutinib is at a dose of 60 mg. In some instances, the pharmaceutically effective amount of tolebrutinib is at a dose of 120 mg. In some instances, the pharmaceutically effective amount of tolebrutinib is administered orally. In some instances, the pharmaceutically effective amount of tolebrutinib is administered daily. In an embodiment, the method further comprises administering a pharmaceutically effective amount of an anti-CD20 antibody to the subject. In some instances, the anti-CD20 antibody dose comprises one or more intravenous infusion (e.g., intravenous (IV) infusion) of an anti-CD20 antibody. The one or more IV infusions can be administered over a period of time (e.g., for at least six months). In some instances the subject is administered the IV infusion at doses that are about a week to about three weeks (e.g., about 1 week, about 2 weeks, or about 3 weeks) apart. In some instances, the anti-CD20 antibody is administered during treatment with tolebrutinib. In some instances, the anti-CD20 antibody is administered separately from treatment with tolebrutinib. In some instances, the anti-CD20 antibody is administered before treatment with tolebrutinib. In some instances, the anti-CD20 antibody is administered after treatment with tolebrutinib.

In some instances, the methods disclosed herein include a method of assessing the efficacy of a treatment of a pharmaceutically effective amount of tolebrutinib in a subject having MS, the method comprising: (a) detecting (i) CXCL10, CXCL13, CD27, NEFL, CCL4, and/or CCL3 in a first biological sample comprising CSF obtained from the subject at a first time point and (ii) CXCL10, CXCL13, CD27, NEFL, CCL4, and/or CCL3 in a second biological sample comprising CSF obtained from the subject at a second time point, wherein the subject is administered one or more doses of the treatment between the first and second time points; and (b) determining a correlation between efficacy of the treatment and CXCL10, CXCL13, CD27, NEFL, CCL4, and/or CCL3 in the second biological sample as compared to CXCL10, CXCL13, CD27, NEFL, CCL4, and/or CCL3 in a sample obtained from an untreated patient, wherein the CXCL10, CXCL13, CD27, NEFL, CCL4, and/or CCL3 in the second biological sample is about the same or decreased as compared to the abundance in the sample from the untreated patient, thereby indicating that the treatment is effective for MS in the subject. In an embodiment, the method further comprises administering a pharmaceutically effective amount of an anti-CD20 antibody to the subject and assessing the efficacy of a treatment of a pharmaceutically effective amount of tolebrutinib and anti-CD20 antibody.

In some instances, the difference between the first time point and the second time point is about 1 month to about two years. In some instances, the pharmaceutically effective amount of tolebrutinib is administered at a dose of about 50 mg to about 130 mg. In some instances, the pharmaceutically effective amount of tolebrutinib is at a dose of 60 mg. In some instances, the pharmaceutically effective amount of tolebrutinib is at a dose of 120 mg. In some instances, the pharmaceutically effective amount of tolebrutinib is administered orally. In some instances, the pharmaceutically effective amount of tolebrutinib is administered daily. In some instances, the anti-CD20 antibody dose comprises one or more intravenous infusion (e.g., intravenous (IV) infusion) of an anti-CD20 antibody. The one or more IV infusions can be administered over a period of time (e.g., for at least six months). In some instances, the subject is administered the IV infusion at doses that are about a week to about three weeks (e.g., about 1 week, about 2 weeks, or about 3 weeks) apart. In some instances, the CXCL10, CXCL13, CD27, NEFL, CCL4, and/or CCL3 comprises CXCL10, CXCL13 RNA, CD27, NEFL, CCL4, and/or CCL3. In some instances, the CXCL10, CXCL13, CD27, NEFL, CCL4, and/or CCL3 RNA is determined by polymerase chain reaction, quantitative polymerase chain reaction, or Northern blot. In some instances, the CXCL10, CXCL13, CD27, NEFL, CCL4, and/or CCL3 comprises CXCL10, CXCL13, CD27, NEFL, CCL4, and/or CCL3 protein. In some instances, the CXCL10, CXCL13, CD27, NEFL, CCL4, and/or CCL3 is determined by flow cytometry or Western blot. In some instances, the method further comprises monitoring the subject for the development of symptoms of MS. In some instances, the method further comprises administering to the subject a treatment for decreasing the rate of progression or decreasing the likelihood or susceptibility of developing MS.

In some instances, the preceding methods include administering additional or increased doses of the pharmaceutically effective amount of tolebrutinib for the subject. In some instances, the preceding methods include administering additional or increased doses of the pharmaceutically effective amount of tolebrutinib and an anti-CD20 therapy for the subject.

In some instances, the subject has received a treatment for MS prior to detecting CXCL10, CXCL13, CD27, NEFL, CCL4, and/or CCL3. In some instances, the subject has been treated previously with an anti-CD20 therapy.

In some instances, the anti-CD20 therapy is ocrelizumab. In some instances, the anti-CD20 therapy is rituximab.

In some instances, the subject comprises one or more brain lesions.

In some instances, the subject is administered an anti-CD20 therapy and/or one or more doses of booster of an anti-CD20 therapy in addition to the pharmaceutically effective amount of tolebrutinib.

In some instances, administering of the pharmaceutically effective amount of tolebrutinib (and potentially with one or more additional therapies, such as an anti-CD20 therapy) results in one or more of: reduced brain lesions; a reduced amount of iron in brain tissue of the subject; and/or a decrease in synaptic density. In some instances, reduced or decrease(d) is compared to a previous amount from the same subject.

In some instances, the MS is relapsing-remitting multiple sclerosis. In some instances, the MS is secondary progressive multiple sclerosis.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims.)

The terms “or a combination thereof” and “or combinations thereof” as used herein refers to any and all permutations and combinations of the listed terms preceding the term. For example, “A, B, C, or combinations thereof” is intended to include at least one of A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

“Or” is used in the inclusive sense, i.e., equivalent to “and/or,” unless the context requires otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

Embodiment 1. A method of treating a subject having multiple sclerosis (MS), the method comprising:
(a) detecting CXCL13 in cerebrospinal fluid (CSF) in the subject; and
(b) administering a pharmaceutically effective amount of tolebrutinib to the subject.
   Embodiment 2. A method of treating a subject having MS, the method comprising:
(a) detecting CXCL13 in a biological sample comprising CSF from the subject;
(b) identifying the subject expressing CXCL13 in the biological sample as having MS; and
(c) administering a pharmaceutically effective amount of tolebrutinib to the subject.
   Embodiment 3. The method of embodiment 1 or 2, wherein the pharmaceutically effective amount of tolebrutinib is at a dose of about 50 mg to about 130 mg.
   Embodiment 4. The method of embodiment 3, wherein the pharmaceutically effective amount of tolebrutinib is at a dose of 60 mg.
   Embodiment 5. The method of any one of the preceding embodiments, wherein the pharmaceutically effective amount of tolebrutinib is administered orally.
   Embodiment 6. The method of any one of the preceding embodiments, wherein the pharmaceutically effective amount of tolebrutinib is administered daily.
   Embodiment 7. A method of identifying a patient as being suitable for participating in a clinical trial for MS, the method comprising:
(a) detecting CXCL13 in a biological sample comprising CSF from the subject; and
(b) identifying the subject expressing CXCL13 in the biological sample, thereby identifying a patient as being suitable for participating in a clinical trial for MS.
   Embodiment 8. A method of diagnosing a subject as having MS, the method comprising:
(a) detecting CXCL13 in a biological sample comprising CSF from the subject; and
(b) identifying the subject expressing CXCL13 in the biological sample as having MS.
   Embodiment 9. A method of identifying a subject having MS as expressing CXCL13 in a biological sample comprising CSF, the method comprising:
(a) detecting CXCL13 in the biological sample; and
(b) identifying the subject having MS expressing CXCL13 in the biological sample.
   Embodiment 10. A method of identifying a subject as having an increased likelihood of developing MS, the method comprising:
(a) detecting CXCL13 in a biological sample comprising CSF from a subject; and
(b) identifying a subject expressing CXCL13 in the biological sample, as having an increased likelihood of developing MS.
   Embodiment 11. A method of identifying a subject as likely to develop MS, the method comprising:
(a) detecting CXCL13 in a biological sample comprising CSF from a subject; and
(b) identifying a subject expressing CXCL13 in the biological sample, as having an increased likelihood of developing MS.
   Embodiment 12. The method of any one of the preceding embodiments, wherein the CXCL13 is increased compared to CXCL13 in a reference sample.
   Embodiment 13. The method of embodiment 12, wherein the reference sample is from a second subject.
   Embodiment 14. The method of embodiment 13, wherein the second subject does not have MS.
   Embodiment 15. The method of any one of embodiments 12-14, wherein the reference sample comprises CSF.
   Embodiment 16. The method of any one of the preceding embodiments, further comprising obtaining the biological sample from the subject.
   Embodiment 17. A method of monitoring progression of MS in a subject over time, the method comprising:
(a) detecting CXCL13 in a first biological sample obtained from a subject at a first time point;
(b) detecting CXCL13 in a second biological sample obtained from the subject at a second time point; and
(c) identifying:
   (i) a subject having increased CXCL13 at the second time point, as compared to CXCL13 at the first time point, as having progressing MS, or
   (ii) a subject having about the same or a decreased CXCL13 at the second time point, as compared to CXCL13 at the first time point, as having static or regressing MS.
      Embodiment 18. The method of embodiments 7-17, further comprising administering a pharmaceutically effective amount of tolebrutinib to the subject.
      Embodiment 19. The method of embodiment 18, wherein the pharmaceutically effective amount of tolebrutinib is at a dose of about 50 mg to about 130 mg.
      Embodiment 20. The method of embodiment 18 or 19, wherein the pharmaceutically effective amount of tolebrutinib is at a dose of 60 mg.
      Embodiment 21. The method of any one of embodiments 7-20, wherein the pharmaceutically effective amount of tolebrutinib is administered orally.
      Embodiment 22. The method of any one of embodiments 7-21, wherein the pharmaceutically effective amount of tolebrutinib is administered daily.
      Embodiment 23. A method of assessing the efficacy of a treatment of a pharmaceutically effective amount of tolebrutinib in a subject having MS, the method comprising:
(a) detecting (i) CXCL13 in a first biological sample comprising CSF obtained from the subject at a first time point and (ii) CXCL13 in a second biological sample comprising CSF obtained from the subject at a second time point, wherein the subject is administered one or more doses of the treatment between the first and second time points; and
(b) determining a correlation between efficacy of the treatment and CXCL13 in the second biological sample as compared to CXCL13 in a sample obtained from an untreated patient, wherein the CXCL13 in the second biological sample is about the same or decreased as compared to the abundance in the sample from the untreated patient, thereby indicating that the treatment is effective for MS in the subject.
   Embodiment 24. The method of embodiment 23, wherein the different between the first time point and the second time point is about 1 month to about two years.
   Embodiment 25. The method of embodiment 23 or 24, wherein the pharmaceutically effective amount of tolebrutinib is administered at a dose of about 50 mg to about 130 mg.
   Embodiment 26. The method of embodiment 25, wherein the pharmaceutically effective amount of tolebrutinib is at a dose of 60 mg.
   Embodiment 27. The method of any one of embodiments 23-26, wherein the pharmaceutically effective amount of tolebrutinib is administered orally.
   Embodiment 28. The method of any one of embodiments 23-27, wherein the pharmaceutically effective amount of tolebrutinib is administered daily.
   Embodiment 29. The method of any one of the preceding embodiments, wherein the CXCL13 comprises CXCL13 RNA.
   Embodiment 30. The method of embodiment 29, wherein CXCL13 RNA is determined by polymerase chain reaction, quantitative polymerase chain reaction, or Northern blot.
   Embodiment 31. The method of any one of the preceding embodiments, wherein the CXCL13 comprises CXCL13 protein.
   Embodiment 32. The method of embodiment 31, wherein the CXCL13 is determined by flow cytometry or Western blot.
   Embodiment 33. The method of any one of the preceding embodiments, wherein the method further comprises monitoring the subject for the development of symptoms of MS.
   Embodiment 34. The method of any one of the preceding embodiments, wherein the method further comprises administering to the subject a treatment for decreasing the rate of progression or decreasing the likelihood or susceptibility of developing MS.
   Embodiment 35. The method of any one of the preceding embodiments, further comprising administering additional or increased doses of the pharmaceutically effective amount of tolebrutinib for the subject.
   Embodiment 36. The method of any one of the preceding embodiments, wherein the subject has received a treatment for MS prior to detecting CXCL13.
   Embodiment 37. The method of embodiment 36, wherein the subject has been treated previously with an anti-CD20 therapy.
   Embodiment 38. The method of embodiment 37, wherein the anti-CD20 therapy is ocrelizumab.
   Embodiment 39. The method of any one of the preceding embodiments, wherein the subject comprises one or more brain lesions.
   Embodiment 40. The method of any one of embodiments 1-6 or 18-39, wherein the subject is administered one or more doses of booster of an anti-CD20 therapy administering the pharmaceutically effective amount of tolebrutinib.
   Embodiment 41. The method of any one of embodiments 1-6 or 18-40, wherein administering of the pharmaceutically effective amount of tolebrutinib results in one or more of:
reduced brain lesions;
a reduced amount of iron in brain tissue of the subject; and/or a decrease in synaptic density.
   Embodiment 42. The method of any one of the preceding embodiments, wherein the MS is relapsing-remitting multiple sclerosis.
   Embodiment 43. The method of any one of embodiments 1-41, wherein the MS is secondary progressive multiple sclerosis.
   Embodiment 44. A method of treating a subject having multiple sclerosis (MS), the method comprising:
(a) detecting CXCL10 in cerebrospinal fluid (CSF) in the subject; and
(b) administering a pharmaceutically effective amount of tolebrutinib to the subject.
   Embodiment 45. A method of treating a subject having MS, the method comprising:
(a) detecting CXCL10 in a biological sample comprising CSF from the subject;
(b) identifying the subject expressing CXCL10 in the biological sample as having MS; and
(c) administering a pharmaceutically effective amount of tolebrutinib to the subject.
   Embodiment 46. The method of embodiment 44 or 45, wherein the pharmaceutically effective amount of tolebrutinib is at a dose of about 50 mg to about 130 mg.
   Embodiment 47. The method of embodiment 46, wherein the pharmaceutically effective amount of tolebrutinib is at a dose of 60 mg.
   Embodiment 48. The method of any one of claims 44-47, wherein the pharmaceutically effective amount of tolebrutinib is administered orally.
   Embodiment 49. The method of any one of claims 44-48, wherein the pharmaceutically effective amount of tolebrutinib is administered daily.
   Embodiment 50. A method of identifying a patient as being suitable for participating in a clinical trial for MS, the method comprising:
(a) detecting CXCL10 in a biological sample comprising CSF from the subject; and
(b) identifying the subject expressing CXCL10 in the biological sample, thereby identifying a patient as being suitable for participating in a clinical trial for MS.
   Embodiment 51. A method of diagnosing a subject as having MS, the method comprising:
(a) detecting CXCL10 in a biological sample comprising CSF from the subject; and
(b) identifying the subject expressing CXCL10 in the biological sample as having MS.
   Embodiment 52. A method of identifying a subject having MS as expressing CXCL10 in a biological sample comprising CSF, the method comprising:
(a) detecting CXCL10 in the biological sample; and
(b) identifying the subject having MS expressing CXCL10 in the biological sample.
   Embodiment 53. A method of identifying a subject as having an increased likelihood of developing MS, the method comprising:
(a) detecting CXCL10 in a biological sample comprising CSF from a subject; and
(b) identifying a subject expressing CXCL10 in the biological sample, as having an increased likelihood of developing MS.
   Embodiment 54. A method of identifying a subject as likely to develop MS, the method comprising:
(a) detecting CXCL10 in a biological sample comprising CSF from a subject; and
(b) identifying a subject expressing CXCL10 in the biological sample, as having an increased likelihood of developing MS.
   Embodiment 55. The method of any one of embodiments 44-55, wherein the CXCL10 is increased compared to CXCL10 in a reference sample.
   Embodiment 56. The method of embodiment 55, wherein the reference sample is from a second subject.
   Embodiment 57. The method of embodiment 56, wherein the second subject does not have MS.
   Embodiment 58. The method of any one of embodiments 55-57, wherein the reference sample comprises CSF.
   Embodiment 59. The method of any one of embodiments 44-58, further comprising obtaining the biological sample from the subject.
   Embodiment 60. A method of monitoring progression of MS in a subject over time, the method comprising:
(a) detecting CXCL10 in a first biological sample obtained from a subject at a first time point;
(b) detecting CXCL10 in a second biological sample obtained from the subject at a second time point; and
(c) identifying:
   (i) a subject having increased CXCL10 at the second time point, as compared to CXCL10 at the first time point, as having progressing MS, or
   (ii) a subject having about the same or a decreased CXCL10 at the second time point, as compared to CXCL10 at the first time point, as having static or regressing MS.
      Embodiment 61. The method of embodiments 50-60, further comprising administering a pharmaceutically effective amount of tolebrutinib to the subject.
      Embodiment 62. The method of embodiment 60, wherein the pharmaceutically effective amount of tolebrutinib is at a dose of about 50 mg to about 130 mg.
      Embodiment 63. The method of embodiment 61 or 62, wherein the pharmaceutically effective amount of tolebrutinib is at a dose of 60 mg.
      Embodiment 64. The method of any one of embodiments 50-63, wherein the pharmaceutically effective amount of tolebrutinib is administered orally.
      Embodiment 65. The method of any one of embodiments 50-64, wherein the pharmaceutically effective amount of tolebrutinib is administered daily.
      Embodiment 66. A method of assessing the efficacy of a treatment of a pharmaceutically effective amount of tolebrutinib in a subject having MS, the method comprising:
(a) detecting (i) CXCL10 in a first biological sample comprising CSF obtained from the subject at a first time point and (ii) CXCL10 in a second biological sample comprising CSF obtained from the subject at a second time point, wherein the subject is administered one or more doses of the treatment between the first and second time points; and
(b) determining a correlation between efficacy of the treatment and CXCL10 in the second biological sample as compared to CXCL10 in a sample obtained from an untreated patient, wherein the CXCL10 in the second biological sample is about the same or decreased as compared to the abundance in the sample from the untreated patient, thereby indicating that the treatment is effective for MS in the subject.
   Embodiment 67. The method of embodiment 66, wherein the different between the first time point and the second time point is about 1 month to about two years.
   Embodiment 68. The method of embodiment 66 or 67, wherein the pharmaceutically effective amount of tolebrutinib is administered at a dose of about 50 mg to about 130 mg.
   Embodiment 69. The method of embodiment 68, wherein the pharmaceutically effective amount of tolebrutinib is at a dose of 60 mg.
   Embodiment 70. The method of any one of embodiments 66-69, wherein the pharmaceutically effective amount of tolebrutinib is administered orally.
   Embodiment 71. The method of any one of embodiments 66-70 wherein the pharmaceutically effective amount of tolebrutinib is administered daily.
   Embodiment 72. The method of any one of embodiments 44-71, wherein the CXCL10 comprises CXCL10 RNA.
   Embodiment 73. The method of embodiment 72, wherein CXCL10 RNA is determined by polymerase chain reaction, quantitative polymerase chain reaction, or Northern blot.
   Embodiment 74. The method of any one of embodiments 44-73, wherein the CXCL10 comprises CXCL10 protein.
   Embodiment 75. The method of embodiment 74, wherein the CXCL10 is determined by flow cytometry or Western blot.
   Embodiment 76. The method of any one of embodiments 44-75, wherein the method further comprises monitoring the subject for the development of symptoms of MS.
   Embodiment 77. The method of any one of embodiments 44-76, wherein the method further comprises administering to the subject a treatment for decreasing the rate of progression or decreasing the likelihood or susceptibility of developing MS.
   Embodiment 78. The method of any one of embodiments 44-77, further comprising administering additional or increased doses of the pharmaceutically effective amount of tolebrutinib for the subject.
   Embodiment 79. The method of any one of embodiments 44-78, wherein the subject has received a treatment for MS prior to detecting CXCL10.
   Embodiment 80. The method of embodiment 79, wherein the subject has been treated previously with an anti-CD20 therapy.
   Embodiment 81. The method of embodiment 80, wherein the anti-CD20 therapy is ocrelizumab.
   Embodiment 82. The method of any one of embodiments 44-81, wherein the subject comprises one or more brain lesions.
   Embodiment 83. The method of any one of embodiments 44-49 or 61-83, wherein the subject is administered one or more doses of booster of an anti-CD20 therapy administering the pharmaceutically effective amount of tolebrutinib.
   Embodiment 84. The method of any one of embodiments 44-49 or 61-83, wherein administering of the pharmaceutically effective amount of tolebrutinib results in one or more of:
reduced brain lesions;
a reduced amount of iron in brain tissue of the subject; and/or
a decrease in synaptic density.
   Embodiment 85. The method of any one of embodiments 44-84, wherein the MS is relapsing-remitting multiple sclerosis.
   Embodiment 86. The method of any one of embodiments 44-85, wherein the MS is secondary progressive multiple sclerosis.
   Embodiment 87. A method of treating a subject having multiple sclerosis (MS), the method comprising:
(a) detecting at least one biomarker in cerebrospinal fluid (CSF) in the subject; and
(b) administering a pharmaceutically effective amount of tolebrutinib to the subject, wherein the at least one biomarker is chosen from CXCL13, CXCL10, CD27, NEFL, CCL4, and CCL3.
   Embodiment 88. A method of treating a subject having MS, the method comprising:
(a) detecting at least one biomarker in a biological sample comprising CSF from the subject;
(b) identifying the subject expressing the at least one biomarker in the biological sample as having MS; and
(c) administering a pharmaceutically effective amount of tolebrutinib to the subject; wherein the at least one biomarker is chosen from CXCL13, CXCL10, CD27, NEFL, CCL4, and CCL3.
   Embodiment 89. The method of embodiment 87 or 88, wherein the pharmaceutically effective amount of tolebrutinib is at a dose of about 50 mg to about 130 mg.
   Embodiment 90. The method of embodiment 89, wherein the pharmaceutically effective amount of tolebrutinib is at a dose of 60 mg.
   Embodiment 91. The method of any one of embodiments 87-90, wherein the pharmaceutically effective amount of tolebrutinib is administered orally.
   Embodiment 92. The method of any one of embodiments 87-91, wherein the pharmaceutically effective amount of tolebrutinib is administered daily.
   Embodiment 93. A method of identifying a patient as being suitable for participating in a clinical trial for MS, the method comprising:
(a) detecting at least one biomarker in a biological sample comprising CSF from the subject; and
(b) identifying the subject expressing the at least one biomarker in the biological sample, thereby identifying a patient as being suitable for participating in a clinical trial for MS;
   wherein the at least one biomarker is chosen from CXCL13, CXCL10, CD27, NEFL, CCL4, and CCL3.
   Embodiment 94. A method of diagnosing a subject as having MS, the method comprising:
(a) detecting at least one biomarker in a biological sample comprising CSF from the subject; and
(b) identifying the subject expressing the at least one biomarker in the biological sample as having MS;
   wherein the at least one biomarker is chosen from CXCL13, CXCL10, CD27, NEFL, CCL4, and CCL3.
   Embodiment 95. A method of identifying a subject having MS as expressing at least one biomarker in a biological sample comprising CSF, the method comprising:
(a) detecting the at least one biomarker in the biological sample; and
(b) identifying the subject having MS expressing the at least one biomarker in the biological sample;
   wherein the at least one biomarker is chosen from CXCL13, CXCL10, CD27, NEFL, CCL4, and CCL3.
   Embodiment 96. A method of identifying a subject as having an increased likelihood of developing MS, the method comprising:
(a) detecting at least one biomarker in a biological sample comprising CSF from a subject; and
(b) identifying a subject expressing the at least one biomarker in the biological sample, as having an increased likelihood of developing MS;
   wherein the at least one biomarker is chosen from CXCL13, CXCL10, CD27, NEFL, CCL4, and CCL3.
   Embodiment 97. A method of identifying a subject as likely to develop MS, the method comprising:
(a) detecting at least one biomarker in a biological sample comprising CSF from a subject; and
(b) identifying a subject expressing the at least one biomarker in the biological sample, as having an increased likelihood of developing MS;
   wherein the at least one biomarker is chosen from CXCL13, CXCL10, CD27, NEFL, CCL4, and CCL3.
   Embodiment 98. The method of any one of embodiments 87-97, wherein the at least one biomarker is increased compared to the at least one biomarker in a reference sample.
   Embodiment 99. The method of embodiment 98, wherein the reference sample is from a second subject.
   Embodiment 100. The method of embodiment 99, wherein the second subject does not have MS.
   Embodiment 101. The method of any one of embodiments 98-100, wherein the reference sample comprises CSF.
   Embodiment 102. The method of any one of embodiments 87-101, further comprising obtaining the biological sample from the subject.
   Embodiment 103. A method of monitoring progression of MS in a subject over time, the method comprising:
(a) detecting at least one biomarker in a first biological sample obtained from a subject at a first time point;
(b) detecting the at least one biomarker in a second biological sample obtained from the subject at a second time point; and
(c) identifying:
   (i) a subject having increased at least one biomarker at the second time point, as compared to the at least one biomarker at the first time point, as having progressing MS, or
   (ii) a subject having about the same or a decreased at least one biomarker at the second time point, as compared to the at least one biomarker at the first time point, as having static or regressing MS;
      wherein the at least one biomarker is chosen from CXCL13, CXCL10, CD27, NEFL, CCL4, and CCL3.
      Embodiment 104. The method of embodiments 93-105, further comprising administering a pharmaceutically effective amount of tolebrutinib to the subject.
      Embodiment 105. The method of embodiment 104, wherein the pharmaceutically effective amount of tolebrutinib is at a dose of about 50 mg to about 130 mg.
      Embodiment 106. The method of embodiment 104 or 105, wherein the pharmaceutically effective amount of tolebrutinib is at a dose of 60 mg.
      Embodiment 107. The method of any one of embodiments 93-106, wherein the pharmaceutically effective amount of tolebrutinib is administered orally.
      Embodiment 108. The method of any one of embodiments 93-107, wherein the pharmaceutically effective amount of tolebrutinib is administered daily.
      Embodiment 109. A method of assessing the efficacy of a treatment of a pharmaceutically effective amount of tolebrutinib in a subject having MS, the method comprising:
(a) detecting (i) at least one biomarker in a first biological sample comprising CSF obtained from the subject at a first time point and (ii) the at least one biomarker in a second biological sample comprising CSF obtained from the subject at a second time point, wherein the subject is administered one or more doses of the treatment between the first and second time points; and
(b) determining a correlation between efficacy of the treatment and the at least one biomarker in the second biological sample as compared to the at least one biomarker in a sample obtained from an untreated patient, wherein the at least one biomarker in the second biological sample is about the same or decreased as compared to the abundance in the sample from the untreated patient, thereby indicating that the treatment is effective for MS in the subject;
   wherein the at least one biomarker is chosen from CXCL13, CXCL10, CD27, NEFL, CCL4, and CCL3.
   Embodiment 110. The method of embodiment 109, wherein the difference between the first time point and the second time point is about 1 month to about two years.
   Embodiment 111. The method of embodiment 109 or 110, wherein the pharmaceutically effective amount of tolebrutinib is administered at a dose of about 50 mg to about 130 mg.
   Embodiment 112. The method of embodiment 111, wherein the pharmaceutically effective amount of tolebrutinib is at a dose of 60 mg.
   Embodiment 113. The method of any one of embodiments 109-112, wherein the pharmaceutically effective amount of tolebrutinib is administered orally.
   Embodiment 114. The method of any one of embodiments 109-113, wherein the pharmaceutically effective amount of tolebrutinib is administered daily.
   Embodiment 115. The method of any one of embodiments 87-114, wherein the at least one biomarker comprises RNA.
   Embodiment 116. The method of embodiment 115, wherein the at least one biomarker RNA is determined by polymerase chain reaction, quantitative polymerase chain reaction, or Northern blot.
   Embodiment 117. The method of any one of embodiments 87-114, wherein the at least one biomarker comprises protein.
   Embodiment 118. The method of embodiment 117, wherein the at least one biomarker is determined by flow cytometry or Western blot.
   Embodiment 119. The method of any one of embodiments 87-118, wherein the method further comprises monitoring the subject for the development of symptoms of MS.
   Embodiment 120. The method of any one of embodiments 87-119, wherein the method further comprises administering to the subject a treatment for decreasing the rate of progression or decreasing the likelihood or susceptibility of developing MS.
   Embodiment 121. The method of any one of embodiments 87-120, further comprising administering additional or increased doses of the pharmaceutically effective amount of tolebrutinib for the subject.
   Embodiment 122. The method of any one of embodiments 87-121, wherein the subject has received a treatment for MS prior to detecting the at least one biomarker.
   Embodiment 123. The method of embodiment 122, wherein the subject has been treated previously with an anti-CD20 therapy.
   Embodiment 124. The method of embodiment 123, wherein the anti-CD20 therapy is ocrelizumab.
   Embodiment 125. The method of any one of embodiments 87-124, wherein the subject comprises one or more brain lesions.
   Embodiment 126. The method of any one of embodiments 87-92 or 94-125, wherein the subject is administered one or more doses of an anti-CD20 therapy in addition to the pharmaceutically effective amount of tolebrutinib.
   Embodiment 127. The method of any one of embodiments 87-92 or 94-126, wherein administering of the pharmaceutically effective amount of tolebrutinib results in one or more of:
reduced brain lesions;
a reduced amount of iron in brain tissue of the subject; and/or
a decrease in synaptic density.
   Embodiment 128. The method of any one of embodiments 87-127, wherein the MS is relapsing-remitting multiple sclerosis.
   Embodiment 129. The method of any one of embodiments 87-128, wherein the MS is secondary progressive multiple sclerosis.
   Embodiment 130. A method of treating a subject having multiple sclerosis (MS), the method comprising:
(a) detecting CD27 in cerebrospinal fluid (CSF) in the subject; and
(b) administering a pharmaceutically effective amount of tolebrutinib to the subject.
   Embodiment 131. A method of treating a subject having MS, the method comprising:
(a) detecting CD27 in a biological sample comprising CSF from the subject;
(b) identifying the subject expressing CD27 in the biological sample as having MS; and
(c) administering a pharmaceutically effective amount of tolebrutinib to the subject.
   Embodiment 132. The method of embodiment 130 or 131, wherein the pharmaceutically effective amount of tolebrutinib is at a dose of about 50 mg to about 130 mg.
   Embodiment 133. The method of embodiment 132, wherein the pharmaceutically effective amount of tolebrutinib is at a dose of 60 mg.
   Embodiment 134. The method of any one of embodiments 130-133, wherein the pharmaceutically effective amount of tolebrutinib is administered orally.
   Embodiment 135. The method of any one of embodiments 130-134, wherein the pharmaceutically effective amount of tolebrutinib is administered daily.
   Embodiment 136. A method of identifying a patient as being suitable for participating in a clinical trial for MS, the method comprising:
(a) detecting CD27 in a biological sample comprising CSF from the subject; and
(b) identifying the subject expressing CD27 in the biological sample, thereby identifying a patient as being suitable for participating in a clinical trial for MS.
   Embodiment 137. A method of diagnosing a subject as having MS, the method comprising:
(a) detecting CD27 in a biological sample comprising CSF from the subject; and
(b) identifying the subject expressing CD27 in the biological sample as having MS.
   Embodiment 138. A method of identifying a subject having MS as expressing CD27 in a biological sample comprising CSF, the method comprising:
(a) detecting CD27 in the biological sample; and
(b) identifying the subject having MS expressing CD27 in the biological sample.
   Embodiment 139. A method of identifying a subject as having an increased likelihood of developing MS, the method comprising:
(a) detecting CD27 in a biological sample comprising CSF from a subject; and
(b) identifying a subject expressing CD27 in the biological sample, as having an increased likelihood of developing MS.
   Embodiment 140. A method of identifying a subject as likely to develop MS, the method comprising:
(a) detecting CD27 in a biological sample comprising CSF from a subject; and
(b) identifying a subject expressing CD27 in the biological sample, as having an increased likelihood of developing MS.
   Embodiment 141. The method of any one of embodiments 130-140, wherein the CD27 is increased compared to CD27 in a reference sample.
   Embodiment 142. The method of embodiment 141, wherein the reference sample is from a second subject.
   Embodiment 143. The method of embodiment 142, wherein the second subject does not have MS.
   Embodiment 144. The method of any one of embodiments 141-143, wherein the reference sample comprises CSF.
   Embodiment 145. The method of any one of embodiments 130-144, further comprising obtaining the biological sample from the subject.
   Embodiment 146. A method of monitoring progression of MS in a subject over time, the method comprising:
(a) detecting CD27 in a first biological sample obtained from a subject at a first time point;
(b) detecting CD27 in a second biological sample obtained from the subject at a second time point; and
(c) identifying:
   (i) a subject having increased CD27 at the second time point, as compared to CD27 at the first time point, as having progressing MS, or
   (ii) a subject having about the same or a decreased CD27 at the second time point, as compared to CD27 at the first time point, as having static or regressing MS.
      Embodiment 147. The method of embodiments 136-146, further comprising administering a pharmaceutically effective amount of tolebrutinib to the subject.
      Embodiment 148. The method of embodiment 147, wherein the pharmaceutically effective amount of tolebrutinib is at a dose of about 50 mg to about 130 mg.
      Embodiment 149. The method of embodiment 147 or 148, wherein the pharmaceutically effective amount of tolebrutinib is at a dose of 60 mg.
      Embodiment 150. The method of any one of embodiments 136-149, wherein the pharmaceutically effective amount of tolebrutinib is administered orally.
      Embodiment 151. The method of any one of embodiments 136-150, wherein the pharmaceutically effective amount of tolebrutinib is administered daily.
      Embodiment 152. A method of assessing the efficacy of a treatment of a pharmaceutically effective amount of tolebrutinib in a subject having MS, the method comprising:
(a) detecting (i) CD27 in a first biological sample comprising CSF obtained from the subject at a first time point and (ii) CD27 in a second biological sample comprising CSF obtained from the subject at a second time point, wherein the subject is administered one or more doses of the treatment between the first and second time points; and
(b) determining a correlation between efficacy of the treatment and CD27 in the second biological sample as compared to CD27 in a sample obtained from an untreated patient, wherein the CD27 in the second biological sample is about the same or decreased as compared to the abundance in the sample from the untreated patient, thereby indicating that the treatment is effective for MS in the subject.
   Embodiment 153. The method of embodiment 152, wherein the difference between the first time point and the second time point is about 1 month to about two years.
   Embodiment 154. The method of embodiment 152 or 153, wherein the pharmaceutically effective amount of tolebrutinib is administered at a dose of about 50 mg to about 130 mg.
   Embodiment 155. The method of embodiment 154, wherein the pharmaceutically effective amount of tolebrutinib is at a dose of 60 mg.
   Embodiment 156. The method of any one of embodiments 152-156, wherein the pharmaceutically effective amount of tolebrutinib is administered orally.
   Embodiment 157. The method of any one of embodiments 152-156, wherein the pharmaceutically effective amount of tolebrutinib is administered daily.
   Embodiment 158. The method of any one of embodiments 130-157, wherein the CD27 comprises CD27 RNA.
   Embodiment 159. The method of embodiment 158, wherein CD27 RNA is determined by polymerase chain reaction, quantitative polymerase chain reaction, or Northern blot.
   Embodiment 160. The method of any one of embodiments 130-157, wherein the CD27 comprises CD27 protein.
   Embodiment 161. The method of embodiment 160, wherein the CD27 is determined by flow cytometry or Western blot.
   Embodiment 162. The method of any one of embodiments 130-161, wherein the method further comprises monitoring the subject for the development of symptoms of MS.
   Embodiment 163. The method of any one of embodiments 130-162, wherein the method further comprises administering to the subject a treatment for decreasing the rate of progression or decreasing the likelihood or susceptibility of developing MS.
   Embodiment 164. The method of any one of embodiments 130-163, further comprising administering additional or increased doses of the pharmaceutically effective amount of tolebrutinib for the subject.
   Embodiment 165. The method of any one of embodiments 130-164, wherein the subject has received a treatment for MS prior to detecting CD27.
   Embodiment 166. The method of embodiment 165, wherein the subject has been treated previously with an anti-CD20 therapy.
   Embodiment 167. The method of embodiment 166, wherein the anti-CD20 therapy is ocrelizumab.
   Embodiment 168. The method of any one of embodiments 130-167, wherein the subject comprises one or more brain lesions.
   Embodiment 169. The method of any one of embodiments 130-135 or 137-168, wherein the subject is administered one or more doses of an anti-CD20 therapy in addition to the pharmaceutically effective amount of tolebrutinib.
   Embodiment 170. The method of any one of embodiments 130-135 or 137-168, wherein administering of the pharmaceutically effective amount of tolebrutinib results in one or more of:
reduced brain lesions;
a reduced amount of iron in brain tissue of the subject; and/or
a decrease in synaptic density.
   Embodiment 171. The method of any one of embodiments 130-170, wherein the MS is relapsing-remitting multiple sclerosis.
   Embodiment 172. The method of any one of embodiments 130-171, wherein the MS is secondary progressive multiple sclerosis.
   Embodiment 173. A method of treating a subject having multiple sclerosis (MS), the method comprising:
(a) detecting NEFL in cerebrospinal fluid (CSF) in the subject; and
(b) administering a pharmaceutically effective amount of tolebrutinib to the subject.
   Embodiment 174. A method of treating a subject having MS, the method comprising:
(a) detecting NEFL in a biological sample comprising CSF from the subject;
(b) identifying the subject expressing NEFL in the biological sample as having MS; and
(c) administering a pharmaceutically effective amount of tolebrutinib to the subject.
   Embodiment 175. The method of embodiment 173 or 174, wherein the pharmaceutically effective amount of tolebrutinib is at a dose of about 50 mg to about 130 mg.
   Embodiment 176. The method of embodiment 175, wherein the pharmaceutically effective amount of tolebrutinib is at a dose of 60 mg.
   Embodiment 177. The method of any one of embodiments 173-176, wherein the pharmaceutically effective amount of tolebrutinib is administered orally.
   Embodiment 178. The method of any one of embodiments 173-177, wherein the pharmaceutically effective amount of tolebrutinib is administered daily.
   Embodiment 179. A method of identifying a patient as being suitable for participating in a clinical trial for MS, the method comprising:
(a) detecting NEFL in a biological sample comprising CSF from the subject; and
(b) identifying the subject expressing NEFL in the biological sample, thereby identifying a patient as being suitable for participating in a clinical trial for MS.
   Embodiment 180. A method of diagnosing a subject as having MS, the method comprising:
(a) detecting NEFL in a biological sample comprising CSF from the subject; and
(b) identifying the subject expressing NEFL in the biological sample as having MS.
   Embodiment 181. A method of identifying a subject having MS as expressing NEFL in a biological sample comprising CSF, the method comprising:
(a) detecting NEFL in the biological sample; and
(b) identifying the subject having MS expressing NEFL in the biological sample.
   Embodiment 182. A method of identifying a subject as having an increased likelihood of developing MS, the method comprising:
(a) detecting NEFL in a biological sample comprising CSF from a subject; and
(b) identifying a subject expressing NEFL in the biological sample, as having an increased likelihood of developing MS.
   Embodiment 183. A method of identifying a subject as likely to develop MS, the method comprising:
(a) detecting NEFL in a biological sample comprising CSF from a subject; and
(b) identifying a subject expressing NEFL in the biological sample, as having an increased likelihood of developing MS.
   Embodiment 184. The method of any one of embodiments 173-183, wherein the NEFL is increased compared to NEFL in a reference sample.
   Embodiment 185. The method of embodiment 184, wherein the reference sample is from a second subject.
   Embodiment 186. The method of embodiment 185, wherein the second subject does not have MS.
   Embodiment 187. The method of any one of embodiments 184-186, wherein the reference sample comprises CSF.
   Embodiment 188. The method of any one of embodiments 173-187, further comprising obtaining the biological sample from the subject.
   Embodiment 189. A method of monitoring progression of MS in a subject over time, the method comprising:
(a) detecting NEFL in a first biological sample obtained from a subject at a first time point;
(b) detecting NEFL in a second biological sample obtained from the subject at a second time point; and
(c) identifying:
   (i) a subject having increased NEFL at the second time point, as compared to NEFL at the first time point, as having progressing MS, or
   (ii) a subject having about the same or a decreased NEFL at the second time point, as compared to NEFL at the first time point, as having static or regressing MS.
      Embodiment 190. The method of embodiments 173-189, further comprising administering a pharmaceutically effective amount of tolebrutinib to the subject.
      Embodiment 191. The method of embodiment 190, wherein the pharmaceutically effective amount of tolebrutinib is at a dose of about 50 mg to about 130 mg.
      Embodiment 192. The method of embodiment 190 or 191, wherein the pharmaceutically effective amount of tolebrutinib is at a dose of 60 mg.
      Embodiment 193. The method of any one of embodiments 179-192, wherein the pharmaceutically effective amount of tolebrutinib is administered orally.
      Embodiment 194. The method of any one of embodiments 179-193, wherein the pharmaceutically effective amount of tolebrutinib is administered daily.
      Embodiment 195. A method of assessing the efficacy of a treatment of a pharmaceutically effective amount of tolebrutinib in a subject having MS, the method comprising:
(a) detecting (i) NEFL in a first biological sample comprising CSF obtained from the subject at a first time point and (ii) NEFL in a second biological sample comprising CSF obtained from the subject at a second time point, wherein the subject is administered one or more doses of the treatment between the first and second time points; and
(b) determining a correlation between efficacy of the treatment and NEFL in the second biological sample as compared to NEFL in a sample obtained from an untreated patient, wherein the NEFL in the second biological sample is about the same or decreased as compared to the abundance in the sample from the untreated patient, thereby indicating that the treatment is effective for MS in the subject.
   Embodiment 196. The method of embodiment 195, wherein the difference between the first time point and the second time point is about 1 month to about two years.
   Embodiment 197. The method of embodiment 195 or 196, wherein the pharmaceutically effective amount of tolebrutinib is administered at a dose of about 50 mg to about 130 mg.
   Embodiment 198. The method of embodiment 197, wherein the pharmaceutically effective amount of tolebrutinib is at a dose of 60 mg.
   Embodiment 199. The method of any one of embodiments 195-198, wherein the pharmaceutically effective amount of tolebrutinib is administered orally.
   Embodiment 200. The method of any one of embodiments 195-199, wherein the pharmaceutically effective amount of tolebrutinib is administered daily.
   Embodiment 201. The method of any one of embodiments 173-200, wherein the NEFL comprises NEFL RNA.
   Embodiment 202. The method of embodiment 201, wherein NEFL RNA is determined by polymerase chain reaction, quantitative polymerase chain reaction, or Northern blot.
   Embodiment 203. The method of any one of embodiments 173-200, wherein the NEFL comprises NEFL protein.
   Embodiment 204. The method of embodiment 203, wherein the NEFL is determined by flow cytometry or Western blot.
   Embodiment 205. The method of any one of embodiments 173-204, wherein the method further comprises monitoring the subject for the development of symptoms of MS.
   Embodiment 206. The method of any one of embodiments 173-205, wherein the method further comprises administering to the subject a treatment for decreasing the rate of progression or decreasing the likelihood or susceptibility of developing MS.
   Embodiment 207. The method of any one of embodiments 173-206, further comprising administering additional or increased doses of the pharmaceutically effective amount of tolebrutinib for the subject.
   Embodiment 208. The method of any one of embodiments 173-207, wherein the subject has received a treatment for MS prior to detecting NEFL.
   Embodiment 209. The method of embodiment 208, wherein the subject has been treated previously with an anti-CD20 therapy.
   Embodiment 210. The method of embodiment 209, wherein the anti-CD20 therapy is ocrelizumab.
   Embodiment 211. The method of any one of embodiments 173-210, wherein the subject comprises one or more brain lesions.
   Embodiment 212. The method of any one of embodiments 173-178 or 180-211, wherein the subject is administered one or more doses of an anti-CD20 therapy in addition to the pharmaceutically effective amount of tolebrutinib.
   Embodiment 213. The method of any one of embodiments 173-178 or 180-212, wherein administering of the pharmaceutically effective amount of tolebrutinib results in one or more of:
reduced brain lesions;
a reduced amount of iron in brain tissue of the subject; and/or
a decrease in synaptic density.
   Embodiment 214. The method of any one of embodiments 173-213, wherein the MS is relapsing-remitting multiple sclerosis.
   Embodiment 215. The method of any one of embodiments 173-214, wherein the MS is secondary progressive multiple sclerosis.
   Embodiment 216. A method of treating a subject having multiple sclerosis (MS), the method comprising:
(a) detecting CCL4 in cerebrospinal fluid (CSF) in the subject; and
(b) administering a pharmaceutically effective amount of tolebrutinib to the subject.
   Embodiment 217. A method of treating a subject having MS, the method comprising:
(a) detecting CCL4 in a biological sample comprising CSF from the subject;
(b) identifying the subject expressing CCL4 in the biological sample as having MS; and
(c) administering a pharmaceutically effective amount of tolebrutinib to the subject.
   Embodiment 218. The method of embodiment 216 or 217, wherein the pharmaceutically effective amount of tolebrutinib is at a dose of about 50 mg to about 130 mg.
   Embodiment 219. The method of embodiment 218, wherein the pharmaceutically effective amount of tolebrutinib is at a dose of 60 mg.
   Embodiment 220. The method of any one of embodiments 216-219, wherein the pharmaceutically effective amount of tolebrutinib is administered orally.
   Embodiment 221. The method of any one of embodiments 216-220, wherein the pharmaceutically effective amount of tolebrutinib is administered daily.
   Embodiment 222. A method of identifying a patient as being suitable for participating in a clinical trial for MS, the method comprising:
(a) detecting CCL4 in a biological sample comprising CSF from the subject; and
(b) identifying the subject expressing CCL4 in the biological sample, thereby identifying a patient as being suitable for participating in a clinical trial for MS.
   Embodiment 223. A method of diagnosing a subject as having MS, the method comprising:
(a) detecting CCL4 in a biological sample comprising CSF from the subject; and
(b) identifying the subject expressing CCL4 in the biological sample as having MS.
   Embodiment 224. A method of identifying a subject having MS as expressing CCL4 in a biological sample comprising CSF, the method comprising:
(a) detecting CCL4 in the biological sample; and
(b) identifying the subject having MS expressing CCL4 in the biological sample.
   Embodiment 225. A method of identifying a subject as having an increased likelihood of developing MS, the method comprising:
(a) detecting CCL4 in a biological sample comprising CSF from a subject; and
(b) identifying a subject expressing CCL4 in the biological sample, as having an increased likelihood of developing MS.
   Embodiment 226. A method of identifying a subject as likely to develop MS, the method comprising:
(a) detecting CCL4 in a biological sample comprising CSF from a subject; and
(b) identifying a subject expressing CCL4 in the biological sample, as having an increased likelihood of developing MS.
   Embodiment 227. The method of any one of embodiments 216-226, wherein the CCL4 is increased compared to CCL4 in a reference sample.
   Embodiment 228. The method of embodiment 227, wherein the reference sample is from a second subject.
   Embodiment 229. The method of embodiment 228, wherein the second subject does not have MS.
   Embodiment 230. The method of any one of embodiments 216-229, wherein the reference sample comprises CSF.
   Embodiment 231. The method of any one of embodiments 216-230, further comprising obtaining the biological sample from the subject.
   Embodiment 232. A method of monitoring progression of MS in a subject over time, the method comprising:
(a) detecting CCL4 in a first biological sample obtained from a subject at a first time point;
(b) detecting CCL4 in a second biological sample obtained from the subject at a second time point; and
(c) identifying:
   (i) a subject having increased CCL4 at the second time point, as compared to CCL4 at the first time point, as having progressing MS, or
   (ii) a subject having about the same or a decreased CCL4 at the second time point, as compared to CCL4 at the first time point, as having static or regressing MS.
      Embodiment 233. The method of embodiments 222-232, further comprising administering a pharmaceutically effective amount of tolebrutinib to the subject.
      Embodiment 234. The method of embodiment 233, wherein the pharmaceutically effective amount of tolebrutinib is at a dose of about 50 mg to about 130 mg.
      Embodiment 235. The method of embodiment 233 or 234, wherein the pharmaceutically effective amount of tolebrutinib is at a dose of 60 mg.
      Embodiment 236. The method of any one of embodiments 222-235, wherein the pharmaceutically effective amount of tolebrutinib is administered orally.
      Embodiment 237. The method of any one of embodiments 222-236, wherein the pharmaceutically effective amount of tolebrutinib is administered daily.
      Embodiment 238. A method of assessing the efficacy of a treatment of a pharmaceutically effective amount of tolebrutinib in a subject having MS, the method comprising:
(a) detecting (i) CCL4 in a first biological sample comprising CSF obtained from the subject at a first time point and (ii) CCL4 in a second biological sample comprising CSF obtained from the subject at a second time point, wherein the subject is administered one or more doses of the treatment between the first and second time points; and
(b) determining a correlation between efficacy of the treatment and CCL4 in the second biological sample as compared to CCL4 in a sample obtained from an untreated patient, wherein the CCL4 in the second biological sample is about the same or decreased as compared to the abundance in the sample from the untreated patient, thereby indicating that the treatment is effective for MS in the subject.
   Embodiment 239. The method of embodiment 238, wherein the difference between the first time point and the second time point is about 1 month to about two years.
   Embodiment 240. The method of embodiment 238 or 239, wherein the pharmaceutically effective amount of tolebrutinib is administered at a dose of about 50 mg to about 130 mg.
   Embodiment 241. The method of embodiment 240, wherein the pharmaceutically effective amount of tolebrutinib is at a dose of 60 mg.
   Embodiment 242. The method of any one of embodiments 238-241, wherein the pharmaceutically effective amount of tolebrutinib is administered orally.
   Embodiment 243. The method of any one of embodiments 238-242, wherein the pharmaceutically effective amount of tolebrutinib is administered daily.
   Embodiment 244. The method of any one of embodiments 216-243, wherein the CCL4 comprises CCL4 RNA.
   Embodiment 245. The method of embodiment 244, wherein CCL4 RNA is determined by polymerase chain reaction, quantitative polymerase chain reaction, or Northern blot.
   Embodiment 246. The method of any one of embodiments 216-243, wherein the CCL4 comprises CCL4 protein.
   Embodiment 247. The method of embodiment 246, wherein the CCL4 is determined by flow cytometry or Western blot.
   Embodiment 248. The method of any one of embodiments 216-247, wherein the method further comprises monitoring the subject for the development of symptoms of MS.
   Embodiment 249. The method of any one of embodiments 216-248, wherein the method further comprises administering to the subject a treatment for decreasing the rate of progression or decreasing the likelihood or susceptibility of developing MS.
   Embodiment 250. The method of any one of embodiments 216-249, further comprising administering additional or increased doses of the pharmaceutically effective amount of tolebrutinib for the subject.
   Embodiment 251. The method of any one of embodiments 216-250, wherein the subject has received a treatment for MS prior to detecting CCL4.
   Embodiment 252. The method of embodiment 251, wherein the subject has been treated previously with an anti-CD20 therapy.
   Embodiment 253. The method of embodiment 252, wherein the anti-CD20 therapy is ocrelizumab.
   Embodiment 254. The method of any one of embodiments 216-253, wherein the subject comprises one or more brain lesions.
   Embodiment 255. The method of any one of embodiments 216-221 or 223-254, wherein the subject is administered one or more doses of an anti-CD20 therapy in addition to the pharmaceutically effective amount of tolebrutinib.
   Embodiment 256. The method of any one of embodiments 216-221 or 223-255, wherein administering of the pharmaceutically effective amount of tolebrutinib results in one or more of:
reduced brain lesions;
a reduced amount of iron in brain tissue of the subject; and/or
a decrease in synaptic density.
   Embodiment 257. The method of any one of embodiments 216-256, wherein the MS is relapsing-remitting multiple sclerosis.
   Embodiment 258. The method of any one of embodiments 216-257, wherein the MS is secondary progressive multiple sclerosis.
   Embodiment 259. A method of treating a subject having multiple sclerosis (MS), the method comprising:
(a) detecting CCL3 in cerebrospinal fluid (CSF) in the subject; and
(b) administering a pharmaceutically effective amount of tolebrutinib to the subject.
   Embodiment 260. A method of treating a subject having MS, the method comprising:
(a) detecting CCL3 in a biological sample comprising CSF from the subject;
(b) identifying the subject expressing CCL3 in the biological sample as having MS; and
(c) administering a pharmaceutically effective amount of tolebrutinib to the subject.
   Embodiment 261. The method of embodiment 259 or 260, wherein the pharmaceutically effective amount of tolebrutinib is at a dose of about 50 mg to about 130 mg.
   Embodiment 262. The method of embodiment 261, wherein the pharmaceutically effective amount of tolebrutinib is at a dose of 60 mg.
   Embodiment 263. The method of any one of embodiments 259-262, wherein the pharmaceutically effective amount of tolebrutinib is administered orally.
   Embodiment 264. The method of any one of embodiments 259-263, wherein the pharmaceutically effective amount of tolebrutinib is administered daily.
   Embodiment 265. A method of identifying a patient as being suitable for participating in a clinical trial for MS, the method comprising:
(a) detecting CCL3 in a biological sample comprising CSF from the subject; and
(b) identifying the subject expressing CCL3 in the biological sample, thereby identifying a patient as being suitable for participating in a clinical trial for MS.
   Embodiment 266. A method of diagnosing a subject as having MS, the method comprising:
(a) detecting CCL3 in a biological sample comprising CSF from the subject; and
(b) identifying the subject expressing CCL3 in the biological sample as having MS.
   Embodiment 267. A method of identifying a subject having MS as expressing CCL3 in a biological sample comprising CSF, the method comprising:
(a) detecting CCL3 in the biological sample; and
(b) identifying the subject having MS expressing CCL3 in the biological sample.
   Embodiment 268. A method of identifying a subject as having an increased likelihood of developing MS, the method comprising:
(a) detecting CCL3 in a biological sample comprising CSF from a subject; and
(b) identifying a subject expressing CCL3 in the biological sample, as having an increased likelihood of developing MS.
   Embodiment 269. A method of identifying a subject as likely to develop MS, the method comprising:
(a) detecting CCL3 in a biological sample comprising CSF from a subject; and
(b) identifying a subject expressing CCL3 in the biological sample, as having an increased likelihood of developing MS.
   Embodiment 270. The method of any one of embodiments 259-269, wherein the CCL4 is increased compared to CCL4 in a reference sample.
   Embodiment 271. The method of embodiment 270, wherein the reference sample is from a second subject.
   Embodiment 272. The method of embodiment 271, wherein the second subject does not have MS.
   Embodiment 273. The method of any one of embodiments 270-272, wherein the reference sample comprises CSF.
   Embodiment 274. The method of any one of embodiments 259-273, further comprising obtaining the biological sample from the subject.
   Embodiment 275. A method of monitoring progression of MS in a subject over time, the method comprising:
(a) detecting CCL3 in a first biological sample obtained from a subject at a first time point;
(b) detecting CCL3 in a second biological sample obtained from the subject at a second time point; and
(c) identifying:
   (i) a subject having increased CCL3 at the second time point, as compared to CCL3 at the first time point, as having progressing MS, or
   (ii) a subject having about the same or a decreased CCL3 at the second time point, as compared to CCL3 at the first time point, as having static or regressing MS.
      Embodiment 276. The method of embodiments 265-275, further comprising administering a pharmaceutically effective amount of tolebrutinib to the subject.
      Embodiment 277. The method of embodiment 276, wherein the pharmaceutically effective amount of tolebrutinib is at a dose of about 50 mg to about 130 mg.
      Embodiment 278. The method of embodiment 276 or 277, wherein the pharmaceutically effective amount of tolebrutinib is at a dose of 60 mg.
      Embodiment 279. The method of any one of embodiments 265-278, wherein the pharmaceutically effective amount of tolebrutinib is administered orally.
      Embodiment 280. The method of any one of embodiments 265-279, wherein the pharmaceutically effective amount of tolebrutinib is administered daily.
      Embodiment 281. A method of assessing the efficacy of a treatment of a pharmaceutically effective amount of tolebrutinib in a subject having MS, the method comprising:
(a) detecting (i) CCL3 in a first biological sample comprising CSF obtained from the subject at a first time point and (ii) CCL3 in a second biological sample comprising CSF obtained from the subject at a second time point, wherein the subject is administered one or more doses of the treatment between the first and second time points; and
(b) determining a correlation between efficacy of the treatment and CCL3 in the second biological sample as compared to CCL3 in a sample obtained from an untreated patient, wherein the CCL3 in the second biological sample is about the same or decreased as compared to the abundance in the sample from the untreated patient, thereby indicating that the treatment is effective for MS in the subject.
   Embodiment 282. The method of embodiment 281, wherein the difference between the first time point and the second time point is about 1 month to about two years.
   Embodiment 283. The method of embodiment 281 or 282, wherein the pharmaceutically effective amount of tolebrutinib is administered at a dose of about 50 mg to about 130 mg.
   Embodiment 284. The method of embodiment 283, wherein the pharmaceutically effective amount of tolebrutinib is at a dose of 60 mg.
   Embodiment 285. The method of any one of embodiments 281-284, wherein the pharmaceutically effective amount of tolebrutinib is administered orally.
   Embodiment 286. The method of any one of embodiments 281-285, wherein the pharmaceutically effective amount of tolebrutinib is administered daily.
   Embodiment 287. The method of any one of embodiments 259-286, wherein the CCL3 comprises CCL3 RNA.
   Embodiment 288. The method of embodiment 287, wherein CCL3 RNA is determined by polymerase chain reaction, quantitative polymerase chain reaction, or Northern blot.
   Embodiment 289. The method of any one of embodiments 259-286, wherein the CCL3 comprises CCL3 protein.
   Embodiment 290. The method of embodiment 289, wherein the CCL3 is determined by flow cytometry or Western blot.
   Embodiment 291. The method of any one of embodiments 259-290, wherein the method further comprises monitoring the subject for the development of symptoms of MS.
   Embodiment 292. The method of any one of embodiments 259-291, wherein the method further comprises administering to the subject a treatment for decreasing the rate of progression or decreasing the likelihood or susceptibility of developing MS.
   Embodiment 293. The method of any one of embodiments 259-292, further comprising administering additional or increased doses of the pharmaceutically effective amount of tolebrutinib for the subject.
   Embodiment 294. The method of any one of embodiments 259-293, wherein the subject has received a treatment for MS prior to detecting CCL3.
   Embodiment 295. The method of embodiment 294, wherein the subject has been treated previously with an anti-CD20 therapy.
   Embodiment 296. The method of embodiment 295, wherein the anti-CD20 therapy is ocrelizumab.
   Embodiment 297. The method of any one of embodiments 259-296, wherein the subject comprises one or more brain lesions.
   Embodiment 298. The method of any one of embodiments 259-264 or 266-297, wherein the subject is administered one or more doses of an anti-CD20 therapy in addition to the pharmaceutically effective amount of tolebrutinib.
   Embodiment 299. The method of any one of embodiments 259-264 or 266-298, wherein administering of the pharmaceutically effective amount of tolebrutinib results in one or more of:
reduced brain lesions;
a reduced amount of iron in brain tissue of the subject; and/or
a decrease in synaptic density.
   Embodiment 300. The method of any one of embodiments 259-299, wherein the MS is relapsing-remitting multiple sclerosis.
   Embodiment 301. The method of any one of embodiments 259-300, wherein the MS is secondary progressive multiple sclerosis.

### BRIEF DESCRIPTION OF THE DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 shows a heatmap of Olink Proteomics dataset for each sample.

FIG. 2A shows differential abundance analysis of untreated multiple sclerosis (MS) subjects (n=71) (left) compared to healthy volunteers (n=31) (right). The wheel-shaped dots are proteins with decreased abundance in untreated MS subjects compared to healthy volunteers. The solid dots are proteins with increased abundance in untreated MS subjects compared to healthy volunteers.

FIG. 2B shows a pathway analysis of proteins differentially abundant in untreated MS subjects. Circle size indicates ratio of genes in dataset relative to pathway size.

FIG. 2C-2E shows examples of proteins elevated in the CSF of subjects with untreated MS (MZB1 (FIG. 2C), CD79B (FIG. 2D), and TNFRSF13B (FIG. 2E)).

FIG. 3 shows differential abundance analysis of MS subjects treated with a B-cell depleting agent compared to untreated MS subjects. The wheel-shaped dots are proteins with decreased abundance in subjects treated with B-cell depleting agent compared to untreated subjects. The solid dots are proteins with increased abundance in subjects treated with B-cell depleting agent compared to untreated subjects.

FIG. 4A shows differential abundance analysis of MS subjects treated with a BTK inhibitor for 48 weeks after transitioning from a B-cell depleting agent (anti-CD20 antibody) (n=6) (left) compared to MS subjects treated with a B-cell depleting agent (anti-CD20 antibody) (n=7) (right). The wheel-shaped dots are proteins with decreased abundance in subjects treated with BTK inhibitor after transitioning from a B-cell depleting agent compared to subjects treated with a B-cell depleting agent.

FIG. 4B shows principal component analysis of Olink Proteomics dataset showing treatment. HV: healthy volunteer; Untreated: untreated MS subjects; anti-CD20: MS subjects treated with anti-CD20 antibody; 12wk BTKi: MS subjects treated with BTK inhibitor for 12 weeks after transitioning from an anti-CD20 antibody; 48wk BTKi: MS subjects treated with BTK inhibitor for 48 weeks after transitioning from an anti-CD20 antibody.

FIG. 4C-4H shows examples of disease-reversed proteins 12 weeks and 48 weeks after transitioning from anti-CD20 therapy to tolebrutinib (NEFL (FIG. 4C), CXCL13 (FIG. 4D), CXCL10 (FIG. 4E), CD27 (FIG. 4F), CCL4 (FIG. 4G), CCL3 (FIG. 4H)).

FIG. 5 shows CXCL13 protein expression in various subjects. HV: health volunteer; HAM/TSP: HTLV-1 associated myelopathy/tropical spastic paraparesis without treatment; MS_untreated: subjects with MS but have not been treated; MS_ocrelizumab: subjects treated only with an anti-CD20 antibody; MS_tolebrutinib: subjects treated with daily doses of tolebrutinib; NPX: protein expression.

FIG. 6 shows CXCL13 protein expression in various subjects. HV: health volunteer; HAM/TSP: HTLV-1 associated myelopathy/tropical spastic paraparesis without treatment; MS_untreated: subjects with MS but have not been treated; MS_ocrelizumab: subjects treated only with an anti-CD20 antibody; MS_tolebrutinib: subjects treated with daily doses of tolebrutinib. This data is measured via MSD.

FIG. 7 shows CXCL10 was downregulated in subjects who switched treatment from ocrelizumab to tolebrutinib and such data suggest that treatment with tolebrutinib, particularly after a course of treatment with ocrelizumab, can lead to decreased protein levels of CXCL10, which is associated with more favorable clinical outcomes, including active lesions. MS_ocrelizumab_baseline: subjects treated only with ocrelizumab for at least 6 months; MS_tolebrutinib_t1: subjects treated with 12 weeks of tolebrutinib; MS_tolebrutinib_t2: subjects treated with 48 weeks of tolebrutinib.

### DETAILED DESCRIPTION

The present disclosure is predicated on the discovery that at least one biomarker is associated with poor prognosis and development of MS. In some instances, the at least one biomarker is chosen from the proteins listed in Tables 1-3. In some instances, the at least one biomarker is chosen from the proteins listed in Table 3. In some instances, the at least one biomarker is chosen from CXCL13, CXCL10, CD27, NEFL, CCL4, and CCL3. In some instances, at least 2 biomarkers, at least 3 biomarkers, at least 4 biomarkers, at least 5 biomarkers, at least 6 biomarkers, or more, are associated with poor prognosis and development of MS. Herein, the methods provided include identification of at least one biomarker. In some instances, the methods include treating a subject having MS by (a) detecting at least one biomarker in CSF in the subject; and (b) administering a pharmaceutically effective amount of tolebrutinib to the subject. In some instances, the methods include detecting at least two biomarkers, at least three biomarkers, at least four biomarkers, at least five biomarkers, at least six biomarkers, or more. In some instances, the methods further include administering one or more additional therapies, such as an anti-CD20 antibody.

In some embodiments, CXCL13 is associated with poor prognosis and development of MS. Chemokine (C-X-C motif) ligand 13 (CXCL13), also known as B lymphocyte chemoattractant (BLC) or B cell-attracting chemokine 1 (BCA-1), is a protein ligand that in humans is encoded by the CXCL13 gene. Legler D F et al., J. Exp. Med. 187 (4): 655-60 (February 1998); Gunn et al., Nature. 391 (6669): 799-803 (February 1998). Herein, the methods provided include identification of CXCL13. In some instances, the methods include treating a subject having MS by (a) detecting CXCL13 in CSF in the subject; and (b) administering a pharmaceutically effective amount of tolebrutinib to the subject. In some instances, the methods further include administering one or more additional therapies, such as an anti-CD20 antibody.

In some embodiments, CXCL10 is associated with poor prognosis and development of MS. C-X-C motif chemokine ligand 10 (CXCL10) also known as Interferon gamma-induced protein 10 (IP-10) or small-inducible cytokine B10 is an 8.7 kDa protein that in humans is encoded by the CXCL10 gene. Luster et al., Nature. 315 (6021): 672-6 (1985); Luster et al., Proceedings of the National Academy of Sciences of the United States of America. 84 (9): 2868-71 (May 1987). Herein, the methods provided include identification of CXCL10. In some instances, the methods include treating a subject having MS by (a) detecting CXCL10 in CSF in the subject; and (b) administering a pharmaceutically effective amount of tolebrutinib to the subject. In some instances, the methods further include administering one or more additional therapies, such as an anti-CD20 antibody.

In some instances, CD27 is associated with poor prognosis and development of MS. Herein, the methods provided include identification of CD27. In some instances, the methods include treating a subject having MS by (a) detecting CD27 in CSF in the subject; and (b) administering a pharmaceutically effective amount of tolebrutinib to the subject. In some instances, the methods further include administering one or more additional therapies, such as an anti-CD20 antibody.

In some instances, NEFL is associated with poor prognosis and development of MS. Herein, the methods provided include identification of NEFL. In some instances, the methods include treating a subject having MS by (a) detecting NEFL in CSF in the subject; and (b) administering a pharmaceutically effective amount of tolebrutinib to the subject. In some instances, the methods further include administering one or more additional therapies, such as an anti-CD20 antibody.

In some instances, CCL4 is associated with poor prognosis and development of MS. Herein, the methods provided include identification of CCL4. In some instances, the methods include treating a subject having MS by (a) detecting CCL4 in CSF in the subject; and (b) administering a pharmaceutically effective amount of tolebrutinib to the subject. In some instances, the methods further include administering one or more additional therapies, such as an anti-CD20 antibody.

In some instances, CCL3 is associated with poor prognosis and development of MS. Herein, the methods provided include identification of CCL3. In some instances, the methods include treating a subject having MS by (a) detecting CCL3 in CSF in the subject; and (b) administering a pharmaceutically effective amount of tolebrutinib to the subject. In some instances, the methods further include administering one or more additional therapies, such as an anti-CD20 antibody.

In some instances, CXCL13, CXCL10, CD27, NEFL, CCL4, and/or CCL3 are associated with poor prognosis and development. In some instances, CXCL13, CXCL10, CD27, NEFL, CCL4, or CCL3 is associated with poor prognosis and development. In some instances, CXCL13, CXCL10, CD27, NEFL, CCL4, and CCL3 are associated with poor prognosis and development. Herein, the methods provided include identification of CXCL13, CXCL10, CD27, NEFL, CCL4, and/or CCL3. In some instances, the methods include treating a subject having MS by (a) detecting CXCL13, CXCL10, CD27, NEFL, CCL4, and/or CCL3 in CSF in the subject; and (b) administering a pharmaceutically effective amount of tolebrutinib to the subject. In some instances, the methods further include administering one or more additional therapies, such as an anti-CD20 antibody.

Herein, the methods provided also include identification of at least one biomarker. In some instances, the at least one biomarker is chosen from the proteins listed in Tables 1-3. In some instances, the at least one biomarker is chosen from the proteins listed in Table 3. In some instances, the at least one biomarker is chosen from CXCL13, CXCL10, CD27, NEFL, CCL4, and CCL3. In some instances, the methods provided also include identification of at least two biomarkers, at least three biomarkers, at least four biomarkers, at least five biomarkers, or at least six biomarkers, or more. Herein, the methods provided also include identification of CXCL13, CXCL10, CD27, NEFL, CCL4, and/or CCL3.

In some instances, the methods include treating a subject having MS by (a) detecting at least one biomarker in CSF in the subject; and (b) administering a pharmaceutically effective amount of tolebrutinib to the subject. In some instances, the biomarker is chosen from Tables 1-3. In some instances, the at least one biomarker is chosen from the proteins listed in Table 3. In some embodiments, the at least one biomarker is chosen from CXCL1, CXCL10 CD27, NEFL, CCL4, and CCL3. In some instances, the methods include detecting at least two biomarkers, at least three biomarkers, at least four biomarkers, at least five biomarkers, at least six biomarkers, or more, in CSF in the subject. In some instances, the methods include treating a subject having MS by (a) detecting CXCL13, CXCL10, CD27, NEFL, CCL4, and/or CCL3 in CSF in the subject; and (b) administering a pharmaceutically effective amount of tolebrutinib to the subject. In some instances, the methods further include administering one or more additional therapies, such as an anti-CD20 antibody.

MS is classified into three clinical phenotypes: relapsing remitting (RRMS), secondary progressive (SPMS), and primary progressive (PPMS) (Lublin et al. (2014) Neurology. 83:278-86). These three phenotypes are further subdivided into active and non-active forms based on the presence or absence of disease activity, defined by the presence of clinical relapses and/or so-called active lesions on a MRI scan. Active MRI lesions are gadolinium-enhancing lesions on T1-weighted scan (T1Gd+) or new T2-weighted lesions/enlarging T2-weighted lesions. Relapsing MS (RMS) forms encompass RRMS and active SPMS, and progressive MS (PMS) forms constitute non-active SPMS and PPMS. The methods disclosed herein evaluate at least one biomarker in each MS subgroup. In some instances, the at least one biomarker is chosen from the proteins listed in Tables 1-3. In some instances, the at least one biomarker is chosen from the proteins listed in Table 3. In some instances, the at least one biomarker is chosen from CXCL13, CXCL10, CD27, NEFL, CCL4, and CCL3. In some instances, the methods disclosed herein evaluate at least two biomarkers, at least three biomarkers, at least four biomarkers, at least five biomarkers, at least six biomarkers, or more, in CSF in the subject. The methods disclosed herein evaluate CXCL13 in each MS subgroup. The methods disclosed herein evaluate CXCL10 in each MS subgroup. The methods disclosed herein evaluate CD27 in each MS subgroup. The methods disclosed herein evaluate NEFL in each MS subgroup. The methods disclosed herein evaluate CCL4 in each MS subgroup. The methods disclosed herein evaluate CCL3 in each MS subgroup. The methods disclosed herein evaluate CXCL13, CXCL10, CD27, NEFL, CCL4, and/or CCL3 in each MS subgroup.

### I. Definitions

As used herein, “the BTK inhibitor,” “the BTK inhibitor compound,” and “the compound”, can refer to tolebrutinib. Tolebrutinib can also be viewed as (R)-1-(1-acryloylpiperidin-3-yl)-4-amino-3-(4-phenoxyphenyl)-1H-imidazo[4,5-c]pyridin-2(3H)-one having the following structure:

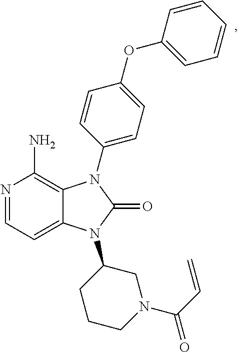 [SMILES: C=CC(=O)N1CCC[C@@H](n2c(=O)n(-c3ccc(Oc4ccccc4)cc3)c3c(N)nccc32)C1]

which is also known as 4-amino-3-(4-phenoxyphenyl)-1-[(3R)-1-(prop-2-enoyl)piperidin-3-yl]-1,3-dihydro-2H-imidazo[4,5-c]pyridin-2-one having the following structure:

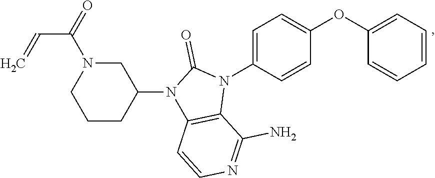 [SMILES: C=CC(=O)N1CCCC(n2c(=O)n(-c3ccc(Oc4ccccc4)cc3)c3c(N)nccc32)C1]

and/or a pharmaceutically acceptable salt thereof.

A “pharmaceutically acceptable carrier” or a “pharmaceutically acceptable excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. A pharmaceutically acceptable carrier/excipient includes both one and more than one such excipient. The pharmaceutically acceptable carrier or adjuvant does not destroy the pharmacological activity thereof and is nontoxic when administered in doses sufficient to deliver a therapeutic amount of the treatment/drug.

In some instances, the pharmaceutical compositions of this disclosure include one or more of acetate, citrate and/or maleate. In some instances, the pharmaceutical compositions can include water or phosphate buffer saline (PBS). In some instance, the pharmaceutical compositions can include chitosan. The pharmaceutical compositions disclosed herein can include one or more pharmaceutically acceptable salts. In some instances, the pharmaceutically acceptable salts include salts comprising hydrochloride, sodium, sulfate, acetate, phosphate or diphosphate, chloride, potassium, maleate, calcium, citrate, mesylate, nitrate, tartrate, aluminum, gluconate, and any combination thereof.

The pharmaceutical compositions of this disclosure may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intra-cutaneous, intra-venous, intra-muscular, intra-articular, intra-arterial, intra-synovial, intra-sternal, intra-thecal, intra-lesional and intra-cranial injection or infusion techniques. In some instances, the pharmaceutical composition is administered orally.

“Treating” or “treatment” of a disease includes: (1) preventing the disease, e.g., causing the clinical symptoms of the disease not to develop in a mammal that may be exposed to or predisposed to the disease but does not yet experience or display symptoms of the disease; (2) inhibiting the disease, e.g., arresting or reducing the development of the disease or its clinical symptoms; and/or (3) relieving the disease, e.g., causing regression of the disease or its clinical symptoms.

“Optional” or “optionally” means that the subsequently described event or circumstance may but need not occur, and that the description includes instances where the event or circumstance occurs and instances in which it does not.

A “therapeutically effective amount” means the amount of the BTK inhibitor compound, that, when administered to a mammal for treating a disease, is sufficient to effect such treatment for the disease. The “therapeutically effective amount” will vary depending on the compound, the disease and its severity and the age, weight, etc., of the mammal to be treated.

A “subject” or “patient” herein is a human subject or patient. Generally, the subject or patient is eligible for treatment for multiple sclerosis. For the purposes herein, such eligible subject or patient is one who is experiencing, has experienced, or is likely to experience, one or more signs, symptoms or other indicators of multiple sclerosis; has been diagnosed with multiple sclerosis, whether, for example, newly diagnosed (with “new onset” MS), previously diagnosed with a new relapse or exacerbation, previously diagnosed and in remission, etc.; and/or is at risk for developing multiple sclerosis. One suffering from or at risk for suffering from multiple sclerosis may optionally be identified as one who has been screened for elevated levels of CD20-positive B cells in serum, cerebrospinal fluid (CSF) and/or MS lesion(s) and/or is screened for using an assay to detect autoantibodies, assessed qualitatively, and preferably quantitatively. Exemplary such autoantibodies associated with multiple sclerosis include anti-myelin basic protein (MBP), anti-myelin oligodendrocytic glycoprotein (MOG), anti-ganglioside and/or anti-neurofilament antibodies. Such autoantibodies may be detected in the subject's serum, cerebrospinal fluid (CSF) and/or MS lesion. By “elevated” autoantibody or B cell level(s) herein is meant level(s) of such autoantibodies or B cells which significantly exceed the level(s) in an individual without MS.

In some embodiments, a subject with MS has at least one documented relapse within the previous year, and/or greater than two documented relapses within the previous two years, and/or greater than one active brain lesion on an MRI scan in the past six months and prior to screening.

In some instances, the methods disclosed herein delay or slow the progression of MS. As used herein, “delaying” or “slowing” the progression of multiple sclerosis means to prevent, defer, hinder, slow, retard, stabilize, and/or postpone development of the disease. This delay can be of varying lengths of time, depending on the history of the disease and/or individual being treated.

In some instances, the subject has one or more symptoms of MS. A “symptom” of MS is any morbid phenomenon or departure from the normal in structure, function, or sensation, experienced by the subject and indicative of MS.

A subject as described herein can have one of a variety of forms of MS. “Multiple sclerosis” (MS) refers to the chronic inflammatory, often disabling disease of the central nervous system characterized by demyelination and neurodegeneration. There are three internationally recognized forms of MS, namely, primary progressive multiple sclerosis (PPMS), relapsing-remitting multiple sclerosis (RRMS), and secondary progressive multiple sclerosis (SPMS).

Within the scope of the present disclosure, “relapsing multiple sclerosis,” “relapsing MS,” or “RMS” may include clinically isolated syndrome (“CIS”), relapsing remitting multiple sclerosis (“RRMS”), and relapsing secondary progressive multiple sclerosis (“R-SPMS.”) See, e.g., Lublin et al., Defining the clinical course of multiple sclerosis; the 2013 revisions, Neurology 2014; 83:278-286.

“Progressive multiple sclerosis” as used herein refers to primary progressive multiple sclerosis (PPMS), and secondary progressive multiple sclerosis (SPMS). In some embodiments, progressive multiple sclerosis is characterized by documented, irreversible loss of neurological function persisting for >6 months that cannot be attributed to clinical relapse.

“Primary progressive multiple sclerosis” or “PPMS” is characterized by a gradual progression of the disease from its onset with rare, superimposed relapses and remissions. There may be periods of a leveling off of disease activity and there may be good and bad days or weeks. PPMS differs from RRMS and SPMS in that onset is typically in the late thirties or early forties, men are as likely women to develop it, and initial disease activity is often in the spinal cord and not in the brain. PPMS disease activity can also be observed (or found) in the brain. PPMS is the sub-type of MS that is least likely to show inflammatory (gadolinium enhancing) lesions on MRI scans. The Primary Progressive form of the disease affects about 15% of all people with multiple sclerosis. PPMS may be defined according to the criteria in Thompson et al. (2018) Lancet 7(2):162-173. The subject with PPMS treated herein is usually one with probable or definitive diagnosis of PPMS. In some embodiments, the multiple sclerosis is primary progressive multiple sclerosis (PPMS). In some embodiments, the patient has been diagnosed PPMS according to the criteria described in Thompson et al. (2018) Lancet Neurol. 17:162-73. In some embodiments, the patient has PPMS, and treatment results in a reduced risk of 12-week composite disability progression (cCDP).

“Relapsing-remitting multiple sclerosis” or “RRMS” is characterized by relapses (also known as exacerbations) during which time new symptoms can appear and old ones resurface or worsen. The relapses are followed by periods of remission, during which time the person fully or partially recovers from the deficits acquired during the relapse. Relapses can last for days, weeks or months and recovery can be slow and gradual or almost instantaneous. The vast majority (about 85%) of people presenting with MS are first diagnosed with RRMS. This is typically when they are in their twenties or thirties, though diagnoses much earlier or later are known. Twice as many women as men present with this sub-type of MS. During relapses, myelin, a protective insulating sheath around the nerve fibers (neurons) in the white matter regions of the central nervous system (CNS), may be damaged in an inflammatory response by the body's own immune system. This causes a wide variety of neurological symptoms that vary considerably depending on which areas of the CNS are damaged. Immediately after a relapse, the inflammatory response dies down and a special type of glial cell in the CNS (called an oligodendrocyte) sponsors remyelination-a process whereby the myelin sheath around the axon may be repaired. It is this remyelination that may be responsible for the remission. Approximately 50% of patients with RRMS convert to SPMS within 10 years of disease onset. After 30 years, this figure rises to 90%. At any one time, the relapsing-remitting form of the disease accounts around 55% of all people with MS.

In some embodiments, the MS is relapsing multiple sclerosis (RMS). In some embodiments, the patient has been diagnosed RMS according to the criteria described in Thompson et al. (2018) Lancet Neurol. 17:162-73. In some embodiments, the patient has RMS, and treatment results in a reduced risk of 12-week composite disability progression (cCDP). In some embodiments, a reduced risk of 12-week cCDP is measured as an increase in the time to onset of cCDP sustained for at least 12 weeks. In some embodiments, time to onset of cCDP refers to the first occurrence of a confirmed progression event according to one of the following three criteria: (i) confirmed disability progression (CDP); (ii) a sustained increase of 20% in Timed 25-Foot Walk Test (T25FWT) score as compared to the T25FWT score at the start of treatment or just prior to the start of treatment (e.g., within any one of 6, 5, 4, 3, 2, or 1 months or any one of 4, 3, 2, or 1 weeks or within 7, 6, 5, 4, 3, 2, or 1 days before the start of treatment); or (iii) a sustained increase of 20% in 9-Hole Peg Test (9-HPT) score as compared to the 9-HPT score at or just prior to the start of treatment (e.g., within any one of 6, 5, 4, 3, 2, or 1 months or any one of 4, 3, 2, or 1 weeks or within 7, 6, 5, 4, 3, 2, or 1 days before the start of treatment). In some embodiment CDP refers to a sustained increase in EDSS score of 1.0 point in a patient with an EDSS score of 5.5 at or just prior to the start of treatment, or a sustained increase in 0.5 points in a patient with an EDSS score of >5.5 at or just prior to the start of treatment.

Also disclosed herein are methods of detecting one or more proteins in a sample (e.g., in a sample comprising CSF). In some instances, the protein is chosen from the proteins listed in Tables 1-3. In some instances, the at least one biomarker is chosen from the proteins listed in Table 3. In some instances, the protein is chosen from CXCL13, CXCL10, CD27, NEFL, CCL4, and CCL3. In some instances, disclosed herein are methods of detecting two or more, three or more, four or more, five or more, or six or more proteins in a sample (e.g., in a sample comprising CSF).

In some instances, the methods include detection of CXCL13. Homeostatic B Cell-Attracting chemokine 1 (BCA-1), otherwise known as CXCL13 (or ANGIE, BLC, BLR1L, ANGIE2, or Scyb13), is constitutively expressed in secondary lymphoid organs (e.g., spleen, lymph nodes, and Peyer's patches) by follicular dendritic cells (FDCs) and macrophages. See Gunn et al., Nature 391:799-803 (1998) and Carlsen et al., Blood 104(10):3021-3027 (2004). CXCL13 primarily acts through G-protein-coupled CXCR5 receptor (Burkitt's lymphoma receptor 1). CXCR5 is expressed, e.g., on mature B lymphocytes, CD4+ follicular helper T cells (Thf cells), a minor subset of CD8+ T cells, and activated tonsillar Treg cells. See Legler et al., J. Exp. Med. 187:655-660 (1998); Forster et al., Blood 84:830-840 (1994); Fazilleau et al., Immunity 30:324-335 (2009); Ansel et al., J. Exp. Med. 190:1123-1134 (1999); Lim et al., J. Clin. Invest. 114(11):1640-1649 (2004); and R. Forster, Chapter in Academic Press Cytokine Reference, August 2000.

CXCL13 is a potent B cell chemoattractant, which directs naïve B cells into the follicles of secondary lymphoid organs and is constitutively expressed by follicular dendritic cells (FDCs) and stromal cells in the B cell rich areas of secondary lymphoid organs. CXCL13 is also known as B cell-attracting chemokine 1 (BCA-1). CXCL13 signals through its receptor CXCR5. CXCR5 is a seven-transmembrane spanning G protein coupled receptor and is a member of the CXC-chemokine receptor subfamily of the class 1 GPCR family. CXCR5 is expressed at high levels on naïve and activated B cells, including peripheral blood and tonsillar B cells. It is also expressed on a subset of activated peripheral blood CD4+ T cells and the majority of CD4+ cells in secondary lymphoid tissue. CXCL13 is the only known ligand for CXCR5.

CXCL13 plays a role in the development of peripheral lymphoid organs; for example, Ansel et al have shown that mice deficient in CXCL13 have severe defects in peripheral lymph node development. CXCL13 induces membrane lymphotoxin α1β2 expression on naïve B cells recruited into follicles, which promotes the maturation of FDCs and further enhances CXCL13 production. CXCL13-deficient mice, immunized with a T cell-dependent antigen, form germinal centers in lymph nodes and spleen but these are small and have an irregular architecture suggesting CXCL13 is required for the recruitment and correct positioning of B cells within follicles.

CXCL13 also has a role in innate immunity; CXCL13-deficient mice lack both peritoneal and pleural cavity B1 cells and are defective in the production of natural antibodies to body cavity bacterial antigens (Ansel, K M. et al. Immunity, 16: 67-76, 2002).

In some instances, the methods include detection of CXCL10. C-X-C motif chemokine ligand 10 (CXCL10) also known as Interferon gamma-induced protein 10 (IP-10) or small-inducible cytokine B10 is an 8.7 kDa protein that in humans is encoded by the CXCL10 gene. Luster et al., Nature. 315 (6021): 672-6 (1985); Luster et al., Proceedings of the National Academy of Sciences of the United States of America. 84 (9): 2868-71 (May 1987).

In some instances, the methods include detection of CD27. In some instances, the methods include detection of NEFL. In some instances, the methods include detection of CCL4. In some instances, the methods include detection of CCL3.

In some instances, the methods include detection of CXCL13, CXCL10, CD27, NEFL, CCL4, and/or CCL3.

### II. Methods of Detection

The methods provided herein include detecting levels of at least one biomarker (e.g., RNA or protein) in the CSF of a subject. In some instances, the at least one biomarker is chosen from the proteins listed in Tables 1-3. In some instances, the at least one biomarker is chosen from the proteins listed in Table 3. In some instances, the at least one biomarker is chosen from CXCL13, CXCL10, CD27, NEFL, CCL4, and CCL3. In some instances, the methods include detecting levels of at least two biomarkers, at least three biomarkers, at least four biomarkers, at least five biomarkers, at least six biomarkers, or more, in CSF in the subject. The methods provided herein include detecting levels of CXCL13 (e.g., RNA or protein) in the CSF of a subject. The methods provided herein include detecting levels of CXCL10 (e.g., RNA or protein) in the CSF of a subject. The methods provided herein include detecting levels of CD27 (e.g., RNA or protein) in the CSF of a subject. The methods provided herein include detecting levels of NEFL (e.g., RNA or protein) in the CSF of a subject. The methods provided herein include detecting levels of CCL4 (e.g., RNA or protein) in the CSF of a subject. The methods provided herein include detecting levels of CCL33 (e.g., RNA or protein) in the CSF of a subject. The methods provided herein include detecting levels of CXCL13, CXCL10, CD27, NEFL, CCL4, and/or CCL3 (e.g., RNA or protein) in the CSF of a subject.

In some instances, the methods include identifying a patient as being suitable for participating in a clinical trial for MS. In some instances, the methods include (a) detecting at least one biomarker, in a biological sample comprising CSF from the subject; and (b) identifying the subject expressing the at least one biomarker, in the biological sample, thereby identifying a patient as being suitable for participating in a clinical trial for MS. In some instances, the methods include detecting at least two biomarkers, at least three biomarkers, at least four biomarkers, at least five biomarkers, at least six, or more biomarkers.

In some instances, the methods include diagnosing a subject as having MS. In some instances, the methods include (a) detecting levels of at least one biomarker, in a biological sample comprising CSF from the subject, and (b) identifying the subject expressing the at least one biomarker in the biological sample, as having MS. In some instances, the methods include detecting at least two biomarkers, at least three biomarkers, at least four biomarkers, at least five biomarkers, at least six, or more, biomarkers.

In some instances, the methods include identifying a subject having MS as expressing at least one biomarker in a biological sample comprising CSF. In some instances, the methods include (a) detecting at least one biomarker, in the biological sample; and (b) identifying the subject having MS expressing the at least one biomarker in the biological sample. In some instances, the methods include detecting at least two biomarkers, at least three biomarkers, at least four biomarkers, at least five biomarkers, at least six, or more, biomarkers.

In some instances, the methods include identifying a subject as having an increased likelihood of developing MS. In some instances, the methods include (a) detecting at least one biomarker in a biological sample comprising CSF from a subject; and (b) identifying a subject expressing the at least one biomarker in the biological sample, as having an increased likelihood of developing MS. In some instances, the methods include detecting at least two biomarkers, at least three biomarkers, at least four biomarkers, at least five biomarkers, at least six, or more biomarkers.

In some instances, the methods include identifying a subject as likely to develop MS. In some instances, the methods include (a) detecting at least one biomarker in a biological sample comprising CSF from a subject; and (b) identifying a subject expressing the at least one biomarker in the biological sample, as having an increased likelihood of developing MS. In some instances, the methods include detecting at least two biomarkers, at least three biomarkers, at least four biomarkers, at least five biomarkers, at least six, or more, biomarkers.

In some instances, the methods include monitoring progression of MS in a subject over time. In some instances, the methods include (a) detecting at least one biomarker in a first biological sample obtained from a subject at a first time point; (b) detecting the at least one biomarker in a second biological sample obtained from the subject at a second time point; and (c) identifying: (i) a subject having increased at least one biomarker at the second time point, as compared to the at least biomarker at the first time point, as having progressing MS, or (ii) a subject having about the same or a decreased at least one biomarker at the second time point, as compared to the at least one biomarker at the first time point, as having static or regressing MS. In some instances, the methods include detecting at least two biomarkers, at least three biomarkers, at least four biomarkers, at least five biomarkers, at least six, or more biomarkers.

In some instances, the methods include assessing the efficacy of a treatment in a subject having MS. In some instances, the treatment comprises a pharmaceutically effective amount of tolebrutinib. In some instances, the treatment comprises a pharmaceutically effective amount of tolebrutinib and a pharmaceutically effective amount of an anti-CD20 antibody. In some instances, the methods include (a) detecting (i) at least one biomarker in a first biological sample comprising CSF obtained from the subject at a first time point and (ii) the at least one biomarker in a second biological sample comprising CSF obtained from the subject at a second time point, wherein the subject is administered one or more doses of the treatment between the first and second time points; and (b) determining a correlation between efficacy of the treatment and the at least one biomarker in the second biological sample as compared to the at least one biomarker in a sample obtained from an untreated patient, wherein the at least one biomarker in the second biological sample is about the same or decreased as compared to the abundance in the sample from the untreated patient, thereby indicating that the treatment is effective for MS in the subject. In some instances, the methods include detecting at least two biomarkers, at least three biomarkers, at least four biomarkers, at least five biomarkers, at least six biomarkers, or more, and determining the correlation between efficacy of the treatment and the biomarker(s).

In some instances, the at least one biomarker is chosen from the proteins listed in Tables 1-3. In some instances, the at least one biomarker is chosen from the proteins listed in Table 3. In some instances, the at least one biomarker is chosen from CXCL13, CXCL10, CD27, NEFL, CCL4, and CCL3.

In some instances, the methods include identifying a patient as being suitable for participating in a clinical trial for MS. In some instances, the methods include (a) detecting CXCL13 in a biological sample comprising CSF from the subject; and (b) identifying the subject expressing CXCL13 in the biological sample, thereby identifying a patient as being suitable for participating in a clinical trial for MS. In some instances, the methods include diagnosing a subject as having MS. In some instances, the methods include (a) detecting CXCL13 in a biological sample comprising CSF from the subject; and (b) identifying the subject expressing CXCL13 in the biological sample as having MS.

In some instances, the methods include identifying a subject having MS as expressing CXCL13 in a biological sample comprising CSF. In some instances, the methods include (a) detecting CXCL13 in the biological sample; and (b) identifying the subject having MS expressing CXCL13 in the biological sample. In some instances, the methods include identifying a subject as having an increased likelihood of developing MS. In some instances, the methods include (a) detecting CXCL13 in a biological sample comprising CSF from a subject; and (b) identifying a subject expressing CXCL13 in the biological sample, as having an increased likelihood of developing MS.

In some instances, the methods include identifying a subject as likely to develop MS. In some instances, the methods include (a) detecting CXCL13 in a biological sample comprising CSF from a subject; and (b) identifying a subject expressing CXCL13 in the biological sample, as having an increased likelihood of developing MS.

In some instances, the methods include monitoring progression of MS in a subject over time. In some instances, the methods include (a) detecting CXCL13 in a first biological sample obtained from a subject at a first time point; (b) detecting CXCL13 in a second biological sample obtained from the subject at a second time point; and (c) identifying: (i) a subject having increased CXCL13 at the second time point, as compared to CXCL13 at the first time point, as having progressing MS, or (ii) a subject having about the same or a decreased CXCL13 at the second time point, as compared to CXCL13 at the first time point, as having static or regressing MS.

In some instances, the methods include assessing the efficacy of a treatment in a subject having MS. In some instances, the treatment comprises a pharmaceutically effective amount of tolebrutinib. In some instances, the treatment comprises a pharmaceutically effective amount of tolebrutinib and a pharmaceutically effective amount of an anti-CD20 antibody. In some instances, the methods include (a) detecting (i) CXCL13 in a first biological sample comprising CSF obtained from the subject at a first time point and (ii) CXCL13 in a second biological sample comprising CSF obtained from the subject at a second time point, wherein the subject is administered one or more doses of the treatment between the first and second time points; and (b) determining a correlation between efficacy of the treatment and CXCL13 in the second biological sample as compared to CXCL13 in a sample obtained from an untreated patient, wherein the CXCL13 in the second biological sample is about the same or decreased as compared to the abundance in the sample from the untreated patient, thereby indicating that the treatment is effective for MS in the subject.

In some instances, the methods include identifying a patient as being suitable for participating in a clinical trial for MS. In some instances, the methods include (a) detecting CXCL10 in a biological sample comprising CSF from the subject; and (b) identifying the subject expressing CXCL10 in the biological sample, thereby identifying a patient as being suitable for participating in a clinical trial for MS. In some instances, the methods include diagnosing a subject as having MS. In some instances, the methods include (a) detecting CXCL10 in a biological sample comprising CSF from the subject; and (b) identifying the subject expressing CXCL10 in the biological sample as having MS.

In some instances, the methods include identifying a subject having MS as expressing CXCL10 in a biological sample comprising CSF. In some instances, the methods include (a) detecting CXCL10 in the biological sample; and (b) identifying the subject having MS expressing CXCL10 in the biological sample. In some instances, the methods include identifying a subject as having an increased likelihood of developing MS. In some instances, the methods include (a) detecting CXCL10 in a biological sample comprising CSF from a subject; and (b) identifying a subject expressing CXCL10 in the biological sample, as having an increased likelihood of developing MS.

In some instances, the methods include identifying a subject as likely to develop MS. In some instances, the methods include (a) detecting CXCL10 in a biological sample comprising CSF from a subject; and (b) identifying a subject expressing CXCL10 in the biological sample, as having an increased likelihood of developing MS.

In some instances, the methods include monitoring progression of MS in a subject over time. In some instances, the methods include (a) detecting CXCL10 in a first biological sample obtained from a subject at a first time point; (b) detecting CXCL10 in a second biological sample obtained from the subject at a second time point; and (c) identifying: (i) a subject having increased CXCL13 at the second time point, as compared to CXCL10 at the first time point, as having progressing MS, or (ii) a subject having about the same or a decreased CXCL13 at the second time point, as compared to CXCL10 at the first time point, as having static or regressing MS.

In some instances, the methods include assessing the efficacy of a treatment in a subject having MS. In some instances, the treatment comprises a pharmaceutically effective amount of tolebrutinib. In some instances, the treatment comprises a pharmaceutically effective amount of tolebrutinib and a pharmaceutically effective amount of an anti-CD20 antibody. In some instances, the methods include (a) detecting (i) CXCL10 in a first biological sample comprising CSF obtained from the subject at a first time point and (ii) CXCL10 in a second biological sample comprising CSF obtained from the subject at a second time point, wherein the subject is administered one or more doses of the treatment between the first and second time points; and (b) determining a correlation between efficacy of the treatment and CXCL10 in the second biological sample as compared to CXCL10 in a sample obtained from an untreated patient, wherein the CXCL10 in the second biological sample is about the same or decreased as compared to the abundance in the sample from the untreated patient, thereby indicating that the treatment is effective for MS in the subject.

In some instances, the methods include identifying a patient as being suitable for participating in a clinical trial for MS. In some instances, the methods include (a) detecting CD27 in a biological sample comprising CSF from the subject; and (b) identifying the subject expressing CD27 in the biological sample, thereby identifying a patient as being suitable for participating in a clinical trial for MS. In some instances, the methods include diagnosing a subject as having MS. In some instances, the methods include (a) detecting CD27 in a biological sample comprising CSF from the subject; and (b) identifying the subject expressing CD27 in the biological sample as having MS.

In some instances, the methods include identifying a subject having MS as expressing CD27 in a biological sample comprising CSF. In some instances, the methods include (a) detecting CD27 in the biological sample; and (b) identifying the subject having MS expressing CD27 in the biological sample. In some instances, the methods include identifying a subject as having an increased likelihood of developing MS. In some instances, the methods include (a) detecting CD27 in a biological sample comprising CSF from a subject; and (b) identifying a subject expressing CD27 in the biological sample, as having an increased likelihood of developing MS.

In some instances, the methods include identifying a subject as likely to develop MS. In some instances, the methods include (a) detecting CD27 in a biological sample comprising CSF from a subject; and (b) identifying a subject expressing CD27 in the biological sample, as having an increased likelihood of developing MS.

In some instances, the methods include monitoring progression of MS in a subject over time. In some instances, the methods include (a) detecting CD27 in a first biological sample obtained from a subject at a first time point; (b) detecting CD27 in a second biological sample obtained from the subject at a second time point; and (c) identifying: (i) a subject having increased CD27 at the second time point, as compared to CD27 at the first time point, as having progressing MS, or (ii) a subject having about the same or a decreased CD27 at the second time point, as compared to CD27 at the first time point, as having static or regressing MS.

In some instances, the methods include assessing the efficacy of a treatment in a subject having MS. In some instances, the treatment comprises a pharmaceutically effective amount of tolebrutinib. In some instances, the treatment comprises a pharmaceutically effective amount of tolebrutinib and a pharmaceutically effective amount of an anti-CD20 antibody. In some instances, the methods include (a) detecting (i) CD27 in a first biological sample comprising CSF obtained from the subject at a first time point and (ii) CD27 in a second biological sample comprising CSF obtained from the subject at a second time point, wherein the subject is administered one or more doses of the treatment between the first and second time points; and (b) determining a correlation between efficacy of the treatment and CD27 in the second biological sample as compared to CD27 in a sample obtained from an untreated patient, wherein the CD27 in the second biological sample is about the same or decreased as compared to the abundance in the sample from the untreated patient, thereby indicating that the treatment is effective for MS in the subject.

In some instances, the methods include identifying a patient as being suitable for participating in a clinical trial for MS. In some instances, the methods include (a) detecting NEFL in a biological sample comprising CSF from the subject; and (b) identifying the subject expressing NEFL in the biological sample, thereby identifying a patient as being suitable for participating in a clinical trial for MS. In some instances, the methods include diagnosing a subject as having MS. In some instances, the methods include (a) detecting NEFL in a biological sample comprising CSF from the subject; and (b) identifying the subject expressing NEFL in the biological sample as having MS.

In some instances, the methods include identifying a subject having MS as expressing NEFL in a biological sample comprising CSF. In some instances, the methods include (a) detecting NEFL in the biological sample; and (b) identifying the subject having MS expressing NEFL in the biological sample. In some instances, the methods include identifying a subject as having an increased likelihood of developing MS. In some instances, the methods include (a) detecting NEFL in a biological sample comprising CSF from a subject; and (b) identifying a subject expressing NEFL in the biological sample, as having an increased likelihood of developing MS.

In some instances, the methods include identifying a subject as likely to develop MS. In some instances, the methods include (a) detecting NEFL in a biological sample comprising CSF from a subject; and (b) identifying a subject expressing NEFL in the biological sample, as having an increased likelihood of developing MS.

In some instances, the methods include monitoring progression of MS in a subject over time. In some instances, the methods include (a) detecting NEFL in a first biological sample obtained from a subject at a first time point; (b) detecting NEFL in a second biological sample obtained from the subject at a second time point; and (c) identifying: (i) a subject having increased NEFL at the second time point, as compared to NEFL at the first time point, as having progressing MS, or (ii) a subject having about the same or a decreased NEFL at the second time point, as compared to NEFL at the first time point, as having static or regressing MS.

In some instances, the methods include assessing the efficacy of a treatment in a subject having MS. In some instances, the treatment comprises a pharmaceutically effective amount of tolebrutinib. In some instances, the treatment comprises a pharmaceutically effective amount of tolebrutinib and a pharmaceutically effective amount of an anti-CD20 antibody. In some instances, the methods include (a) detecting (i) NEFL in a first biological sample comprising CSF obtained from the subject at a first time point and (ii) NEFL in a second biological sample comprising CSF obtained from the subject at a second time point, wherein the subject is administered one or more doses of the treatment between the first and second time points; and (b) determining a correlation between efficacy of the treatment and NEFL in the second biological sample as compared to NEFL in a sample obtained from an untreated patient, wherein the NEFL in the second biological sample is about the same or decreased as compared to the abundance in the sample from the untreated patient, thereby indicating that the treatment is effective for MS in the subject.

In some instances, the methods include identifying a patient as being suitable for participating in a clinical trial for MS. In some instances, the methods include (a) detecting CCL4 in a biological sample comprising CSF from the subject; and (b) identifying the subject expressing CCL4 in the biological sample, thereby identifying a patient as being suitable for participating in a clinical trial for MS. In some instances, the methods include diagnosing a subject as having MS. In some instances, the methods include (a) detecting CCL4 in a biological sample comprising CSF from the subject; and (b) identifying the subject expressing CCL4 in the biological sample as having MS.

In some instances, the methods include identifying a subject having MS as expressing CCL4 in a biological sample comprising CSF. In some instances, the methods include (a) detecting CCL4 in the biological sample; and (b) identifying the subject having MS expressing CCL4 in the biological sample. In some instances, the methods include identifying a subject as having an increased likelihood of developing MS. In some instances, the methods include (a) detecting CCL4 in a biological sample comprising CSF from a subject; and (b) identifying a subject expressing CCL4 in the biological sample, as having an increased likelihood of developing MS.

In some instances, the methods include identifying a subject as likely to develop MS. In some instances, the methods include (a) detecting CCL4 in a biological sample comprising CSF from a subject; and (b) identifying a subject expressing CCL4 in the biological sample, as having an increased likelihood of developing MS.

In some instances, the methods include monitoring progression of MS in a subject over time. In some instances, the methods include (a) detecting CCL4 in a first biological sample obtained from a subject at a first time point; (b) detecting CCL4 in a second biological sample obtained from the subject at a second time point; and (c) identifying: (i) a subject having increased CCL4 at the second time point, as compared to CCL4 at the first time point, as having progressing MS, or (ii) a subject having about the same or a decreased CCL4 at the second time point, as compared to CCL4 at the first time point, as having static or regressing MS.

In some instances, the methods include assessing the efficacy of a treatment in a subject having MS. In some instances, the treatment comprises a pharmaceutically effective amount of tolebrutinib. In some instances, the treatment comprises a pharmaceutically effective amount of tolebrutinib and a pharmaceutically effective amount of an anti-CD20 antibody. In some instances, the methods include (a) detecting (i) CCL4 in a first biological sample comprising CSF obtained from the subject at a first time point and (ii) CCL4 in a second biological sample comprising CSF obtained from the subject at a second time point, wherein the subject is administered one or more doses of the treatment between the first and second time points; and (b) determining a correlation between efficacy of the treatment and CCL4 in the second biological sample as compared to CCL4 in a sample obtained from an untreated patient, wherein the CCL4 in the second biological sample is about the same or decreased as compared to the abundance in the sample from the untreated patient, thereby indicating that the treatment is effective for MS in the subject.

In some instances, the methods include identifying a patient as being suitable for participating in a clinical trial for MS. In some instances, the methods include (a) detecting CCL3 in a biological sample comprising CSF from the subject; and (b) identifying the subject expressing CCL3 in the biological sample, thereby identifying a patient as being suitable for participating in a clinical trial for MS. In some instances, the methods include diagnosing a subject as having MS. In some instances, the methods include (a) detecting CCL3 in a biological sample comprising CSF from the subject; and (b) identifying the subject expressing CCL3 in the biological sample as having MS.

In some instances, the methods include identifying a subject having MS as expressing CCL3 in a biological sample comprising CSF. In some instances, the methods include (a) detecting CCL3 in the biological sample; and (b) identifying the subject having MS expressing CCL3 in the biological sample. In some instances, the methods include identifying a subject as having an increased likelihood of developing MS. In some instances, the methods include (a) detecting CCL3 in a biological sample comprising CSF from a subject; and (b) identifying a subject expressing CCL3 in the biological sample, as having an increased likelihood of developing MS.

In some instances, the methods include identifying a subject as likely to develop MS. In some instances, the methods include (a) detecting CCL3 in a biological sample comprising CSF from a subject; and (b) identifying a subject expressing CCL3 in the biological sample, as having an increased likelihood of developing MS.

In some instances, the methods include monitoring progression of MS in a subject over time. In some instances, the methods include (a) detecting CCL3 in a first biological sample obtained from a subject at a first time point; (b) detecting CCL3 in a second biological sample obtained from the subject at a second time point; and (c) identifying: (i) a subject having increased CCL3 at the second time point, as compared to CCL3 at the first time point, as having progressing MS, or (ii) a subject having about the same or a decreased CCL3 at the second time point, as compared to CCL3 at the first time point, as having static or regressing MS.

In some instances, the methods include assessing the efficacy of a treatment in a subject having MS. In some instances, the treatment comprises a pharmaceutically effective amount of tolebrutinib. In some instances, the treatment comprises a pharmaceutically effective amount of tolebrutinib and a pharmaceutically effective amount of an anti-CD20 antibody. In some instances, the methods include (a) detecting (i) CCL3 in a first biological sample comprising CSF obtained from the subject at a first time point and (ii) CCL3 in a second biological sample comprising CSF obtained from the subject at a second time point, wherein the subject is administered one or more doses of the treatment between the first and second time points; and (b) determining a correlation between efficacy of the treatment and CCL3 in the second biological sample as compared to CCL3 in a sample obtained from an untreated patient, wherein the CCL3 in the second biological sample is about the same or decreased as compared to the abundance in the sample from the untreated patient, thereby indicating that the treatment is effective for MS in the subject.

In some instances, the methods include identifying a patient as being suitable for participating in a clinical trial for MS. In some instances, the methods include (a) detecting CXCL13, CXCL10, CD27, NEFL, CCL4, and/or CCL3 in a biological sample comprising CSF from the subject; and (b) identifying the subject expressing CXCL13, CXCL10, CD27, CCL4, AND/OR CCL3 in the biological sample, thereby identifying a patient as being suitable for participating in a clinical trial for MS. In some instances, the methods include diagnosing a subject as having MS. In some instances, the methods include (a) detecting CXCL13, CXCL10, CD27, CCL4, AND/OR CCL3 in a biological sample comprising CSF from the subject; and (b) identifying the subject expressing CXCL13, CXCL10, CD27, CCL4, AND/OR CCL3 in the biological sample as having MS.

In some instances, the methods include identifying a subject having MS as expressing CXCL13, CXCL10, CD27, CCL4, AND/OR CCL3 in a biological sample comprising CSF. In some instances, the methods include (a) detecting CXCL13, CXCL10, CD27, CCL4, AND/OR CCL3 in the biological sample; and (b) identifying the subject having MS expressing CXCL13, CXCL10, CD27, CCL4, AND/OR CCL3 in the biological sample. In some instances, the methods include identifying a subject as having an increased likelihood of developing MS. In some instances, the methods include (a) detecting CXCL13, CXCL10, CD27, CCL4, AND/OR CCL3 in a biological sample comprising CSF from a subject; and (b) identifying a subject expressing CXCL13, CXCL10, CD27, CCL4, AND/OR CCL3 in the biological sample, as having an increased likelihood of developing MS.

In some instances, the methods include identifying a subject as likely to develop MS. In some instances, the methods include (a) detecting CXCL13, CXCL10, CD27, CCL4, AND/OR CCL3 in a biological sample comprising CSF from a subject; and (b) identifying a subject expressing CXCL13, CXCL10, CD27, CCL4, AND/OR CCL3 in the biological sample, as having an increased likelihood of developing MS.

In some instances, the methods include monitoring progression of MS in a subject over time. In some instances, the methods include (a) detecting CXCL13, CXCL10, CD27, CCL4, AND/OR CCL3 in a first biological sample obtained from a subject at a first time point; (b) detecting CXCL13, CXCL10, CD27, CCL4, AND/OR CCL3 in a second biological sample obtained from the subject at a second time point; and (c) identifying: (i) a subject having increased CXCL13, CXCL10, CD27, CCL4, AND/OR CCL3 at the second time point, as compared to CXCL13, CXCL10, CD27, CCL4, AND/OR CCL3 at the first time point, as having progressing MS, or (ii) a subject having about the same or a decreased CXCL13, CXCL10, CD27, CCL4, AND/OR CCL3 at the second time point, as compared to CXCL13, CXCL10, CD27, CCL4, AND/OR CCL3 at the first time point, as having static or regressing MS.

In some instances, the methods include assessing the efficacy of a treatment in a subject having MS. In some instances, the treatment comprises a pharmaceutically effective amount of tolebrutinib. In some instances, the treatment comprises a pharmaceutically effective amount of tolebrutinib and a pharmaceutically effective amount of an anti-CD20 antibody. In some instances, the methods include (a) detecting (i) CXCL13, CXCL10, CD27, CCL4, AND/OR CCL3 in a first biological sample comprising CSF obtained from the subject at a first time point and (ii) CXCL13, CXCL10, CD27, CCL4, AND/OR CCL3 in a second biological sample comprising CSF obtained from the subject at a second time point, wherein the subject is administered one or more doses of the treatment between the first and second time points; and (b) determining a correlation between efficacy of the treatment and CXCL13, CXCL10, CD27, CCL4, AND/OR CCL3 in the second biological sample as compared to CXCL13, CXCL10, CD27, CCL4, AND/OR CCL3 in a sample obtained from an untreated patient, wherein the CXCL13, CXCL10, CD27, CCL4, AND/OR CCL3 in the second biological sample is about the same or decreased as compared to the abundance in the sample from the untreated patient, thereby indicating that the treatment is effective for MS in the subject.

The methods provided herein include methods of extracting the sample from the subject. In some instances, the methods include extracting CSF from a subject. Methods of extracting CSF from a subject include a lumbar puncture (i.e., a spinal tap) procedure, in which a needle is inserted into the spinal canal to collect cerebrospinal fluid for testing.

In some instances, the methods detect protein (e.g., CXCL13 protein, CXCL10 protein, CD27 protein, NEFL protein, CCL4 protein, and/or CCL3 protein) abundance. In some instances, the methods detect RNA (e.g., CXCL13 RNA, CXCL10 RNA, CD27 RNA, NEFL RNA, CCL4 RNA, and/or CCL3 RNA) abundance. Collectively, proteins and nucleic acids (e.g., RNA) can be called analytes. The present disclosure and methods described herein can be used to detect and analyze a wide variety of different analytes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte can be an organelle (e.g., nuclei or mitochondria). In some instances, the non-nucleic acid analyte is a CXCL13 protein. In some instances, the non-nucleic acid analyte is a CXCL10 protein. In some instances, the non-nucleic acid analyte is a CD27 protein. In some instances, the non-nucleic acid analyte is a NEFL protein. In some instances, the non-nucleic acid analyte is a CCL4 protein. In some instances, the non-nucleic acid analyte is a CCL3 protein. In some instances, the non-nucleic acid analyte is a CXCL10 protein, CXCL13 protein, CD27 protein, NEFL protein, CCL4 protein, and/or CCL4 protein.

Cell surface features corresponding to analytes can include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis.

Proteomics has recently emerged and has been developed for the large-scale study of protein patterns in organisms. Typical goals for proteomic analysis are identification and quantification of proteins present in a specific tissue under specific circumstances. Proteomic technologies, in combination with bioinformatics, are powerful tools for proteins identification and characterization. Commonly, two-dimensional (2D) electrophoresis is used for proteins separation and Mass Spectrometry followed by databank searching are used for protein identification. Up to 10000 proteins can be studied simultaneously. In some instances, Olink Proteomics (www.olink.com) is used to evaluate the proteome. See also Deutsch et al., J Proteome Res. 2021 Dec. 3; 20(12):5241-5263; and Cui et al., Lab Invest, 2022 November; 102(11):1170-1181, each of which is incorporated by reference in its entirety.

The detection and quantitation of individual proteins is one of the fundamental aspects of proteomics. Immunological-based methods such as quantitative enzyme-linked immunosorbent assays (ELISA), Western blotting and dot blotting are very common and sensitive assays for protein detection, and they use antibodies that react specifically with entire proteins or specific epitopes (e.g., fusion tags) after cell lysis. Detection techniques are typically based on chemiluminescence or fluorescence.

Examples of the measurement methods to evaluate protein expression (e.g., CXCL13 protein expression) include LC-MS, immunoassay, enzymatic activity assay, and capillary electrophoresis. In some instances, a qualitative or quantitative approach can be used, which includes: LC-MS; and enzyme immunoassay, two-antibody sandwich ELISA, gold colloid method, radioimmunoassay, latex agglutination immunoassay, fluorescent immunoassay, Western blot, immunohistochemical method, surface plasmon resonance spectroscopy (SPR method), and quartz crystal microbalance (QCM) method, using a monoclonal or polyclonal antibody specific for CXCL13, CXCL10, CD27, NEFL, CCL4, and/or CCL3.

As another example, in some embodiments, one or more immunofluorescence stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for staining and destaining are described, for example, in Bolognesi et al., J. Histochem. Cytochem. 2017; 65(8): 431-444, Lin et al., Nat Commun. 2015; 6:8390, Pirici et al., J Histochem. Cytochem. 2009; 57:567-75, and Glass et al., J. Histochem. Cytochem. 2009; 57:899-905, the entire contents of each of which are incorporated herein by reference.

In some embodiments, immunofluorescence or immunohistochemistry protocols (direct and indirect staining techniques) can be performed as a part of, or in addition to, the exemplary workflows presented herein. For example, samples can be fixed, and analytes (e.g., proteins) are probed using immunofluorescence protocols. For example, the sample can be rehydrated, blocked, and permeabilized (3×SSC, 2% BSA, 0.1% Triton X, 1 U/μl RNAse inhibitor for 10 min at 4° C.) before being stained with fluorescent primary antibodies (1:100 in 3×SSC, 2% BSA, 0.1% Triton X, 1 U/pl RNAse inhibitor for 30 min at 4° C.). The biological sample can be washed, coverslipped (in glycerol+1 U/μl RNAse inhibitor), imaged (e.g., using a confocal microscope or other apparatus capable of fluorescent detection), washed, and processed.

Any variety of staining and imaging techniques as described herein or known in the art can be used in accordance with methods described herein. In some embodiments, the staining includes optical labels as described herein, including, but not limited to, fluorescent, radioactive, chemiluminescent, calorimetric, or colorimetric detectable labels. In some embodiments, the staining includes a fluorescent antibody directed to a target analyte (e.g., cell surface or intracellular proteins) in the biological sample. In some embodiments, the staining includes an immunohistochemistry stain directed to a target analyte (e.g., cell surface or intracellular proteins) in the biological sample. In some embodiments, the staining includes a chemical stain such as hematoxylin and eosin (H&E) or periodic acid-schiff (PAS).

Examples of nucleic acid analytes include DNA analytes such as genomic DNA, methylated DNA, specific methylated DNA sequences, fragmented DNA, mitochondrial DNA, in situ synthesized PCR products, and RNA/DNA hybrids. In some instances, the nucleic acid analyte is a CXCL13 DNA molecule. In some instances, the nucleic acid analyte is a CXCL13 RNA molecule. In some instances, the nucleic acid analyte is a CXCL10 DNA molecule. In some instances, the nucleic acid analyte is a CXCL10 RNA molecule. In some instances, the nucleic acid analyte is a CD27 DNA molecule. In some instances, the nucleic acid analyte is a CD27 RNA molecule. In some instances, the nucleic acid analyte is a NEFL DNA molecule. In some instances, the nucleic acid analyte is a NEFL RNA molecule. In some instances, the nucleic acid analyte is a CCL4 DNA molecule. In some instances, the nucleic acid analyte is a CCL4 RNA molecule. In some instances, the nucleic acid analyte is a CCL3 DNA molecule. In some instances, the nucleic acid analyte is a CCL3 RNA molecule.

Examples of nucleic acid analytes also include RNA analytes such as various types of coding and non-coding RNA. Examples of the different types of RNA analytes include messenger RNA (mRNA), ribosomal RNA (rRNA), transfer RNA (tRNA), microRNA (miRNA), and viral RNA. The RNA can be a transcript (e.g., present in a tissue section). The RNA can be small (e.g., less than 200 nucleic acid bases in length) or large (e.g., RNA greater than 200 nucleic acid bases in length). Small RNAs mainly include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

Detection of at least one biomarker, such as CXCL13 RNA, CXCL10 RNA, CD27 RNA, NEFL RNA, CCL4 RNA, and/or CCL3 RNA, can be performed by methods known in the art. For instance, CXCL13 RNA, CXCL10 RNA, CD27 RNA, NEFL RNA, CCL4 RNA, and/or CCL3 RNA abundance can be determined using qPCR. In some embodiments, the quantification of RNA and/or DNA is carried out by real-time PCR (also known as quantitative PCR or qPCR), using techniques well known in the art, such as but not limited to “TAQMAN™”, or dyes such as “SYBR®”, or on capillaries (“LightCycler® Capillaries”). In some embodiments, the quantification of genetic material is determined by optical absorbance and with real-time PCR. In some embodiments, the quantification of genetic material is determined by digital PCR. In some embodiments, the genes analyzed can be compared to a reference nucleic acid extract (DNA and RNA) corresponding to the expression (mRNA) and quantity (DNA) in order to compare expression levels of the target nucleic acids.

A “PCR amplification” refers to the use of a polymerase chain reaction (PCR) to generate copies of genetic material, including DNA and RNA sequences. Suitable reagents and conditions for implementing PCR are described, for example, in U.S. Pat. Nos. 4,683,202, 4,683,195, 4,800,159, 4,965,188, and 5,512,462, the entire contents of each of which are incorporated herein by reference. In a typical PCR amplification, the reaction mixture includes the genetic material to be amplified, an enzyme, one or more primers that are employed in a primer extension reaction, and reagents for the reaction. The oligonucleotide primers are of sufficient length to provide for hybridization to complementary genetic material under annealing conditions. The length of the primers generally depends on the length of the amplification domains, but will typically be at least 4 bases, at least 5 bases, at least 6 bases, at least 8 bases, at least 9 bases, at least 10 base pairs (bp), at least 11 bp, at least 12 bp, at least 13 bp, at least 14 bp, at least 15 bp, at least 16 bp, at least 17 bp, at least 18 bp, at least 19 bp, at least 20 bp, at least 25 bp, at least 30 bp, at least 35 bp, and can be as long as 40 bp or longer, where the length of the primers will generally range from 18 to 50 bp. The genetic material can be contacted with a single primer or a set of two primers (forward and reverse primers), depending upon whether primer extension, linear or exponential amplification of the genetic material is desired.

In some embodiments, PCR amplification can include reactions such as, but not limited to, a strand-displacement amplification reaction, a rolling circle amplification reaction, a ligase chain reaction, a transcription-mediated amplification reaction, an isothermal amplification reaction, and/or a loop-mediated amplification reaction.

In some instances, sequencing can be performed to determine abundance of at least one biomarker RNA, such as CXCL13 RNA, CXCL10 RNA, CD27 RNA, NEFL RNA, CCL4 RNA, and/or CCL3 RNA. Sequencing of polynucleotides can be performed by various systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based single plex methods, emulsion PCR), and/or isothermal amplification. Non-limiting examples of methods for sequencing genetic material include, but are not limited to, DNA hybridization methods (e.g., Southern blotting), restriction enzyme digestion methods, Sanger sequencing methods, next-generation sequencing methods (e.g., single-molecule real-time sequencing, nanopore sequencing, and Polony sequencing), ligation methods, and microarray methods.

In some embodiments, genetic material is amplified by reverse transcription polymerase chain reaction (RT-PCR). The desired reverse transcriptase activity can be provided by one or more distinct reverse transcriptase enzymes (i.e., RNA dependent DNA polymerases), suitable examples of which include, but are not limited to. M-MLV, MuLV, AMV, HIV, ArrayScript™, MultiScribe™, ThermoScript™, and SuperScript® I, II, III, and IV enzymes. “Reverse transcriptase” includes not only naturally occurring enzymes, but all such modified derivatives thereof, including also derivatives of naturally-occurring reverse transcriptase enzymes.

In some instances, at least one biomarker expression, such as CXCL13, CXCL10, CD27, NEFL, CCL4, and/or CCL3 expression, (RNA or protein) is measured and compared to a reference sample or reference amount. In some instances, CXCL10, CXCL13, CD27, NEFL, CCL4, and/or CCL3 expression (RNA or protein) is measured and compared to a reference sample or reference amount. A reference amount of a nucleic acid/protein can be any appropriate reference amount. In some embodiments, a reference amount of a nucleic acid/protein can be determined based on an amount of the nucleic acid/protein in a corresponding sample (e.g., a reference sample such as a control subject not diagnosed with MS, not presenting with any of the symptoms of MS, not having a family history of MS, and not having any known risk factors of MS) at a corresponding position. In some embodiments, a reference amount of a nucleic acid/protein can be a composite or averaged amount (e.g., the averaged amount of a population of persons having or not having MS).

In some embodiments, a reference amount can be based on a reference amount as published by an appropriate body (e.g., a government agency (e.g., the United States Food and Drug Administration) or a professional organization (e.g., the American Medical Association or American Psychiatric Association)), for example, a reference amount that is a threshold amount for a nucleic acid/protein at the location in the tissue of a subject.

In some embodiments, a reference amount of a nucleic acid/protein can be determined based on any appropriate criteria. For example, in some embodiments, a reference amount of a nucleic acid/protein can come from an age-matched healthy subject. In some embodiments, a reference amount of a nucleic acid/protein can come from a sex-matched healthy subject or a sex-matched healthy subject population. In some embodiments, a reference amount of a nucleic acid/protein can come from an age-matched, sex-matched healthy subject or an age-matched, sex-matched healthy subject population. In some embodiments, a reference amount of a nucleic acid/protein can come from an aggregate sample (e.g., an average of 2 or more individual) of healthy subjects (e.g., that are age-matched and/or sex-matched).

A healthy subject can be any appropriate healthy subject. In some embodiments, a healthy subject does not have MS, does not have symptoms of MS, does not have a genetic mutation associated with MS, does not have a family medical history of MS, no behavior risk factors of MS, or combinations thereof. For example, in some embodiments, a healthy subject has one or more of no known brain disorder, no presentation of symptoms, or no more than three (e.g., no more than two, or no more than one) of: a brain disorder, no known genetic mutations associated with risk of a brain disorder, no family medical history of a brain disorder, and no behavioral risk factors of a brain disorder. Other non-limiting examples of healthy subjects are those that do not have a disorder of a biological system of interest (e.g., circulatory system, digestive and excretory system, endocrine system, integumentary or exocrine system, immune and lymphatic system, muscular system, nervous system, see the brain example above, renal and urinary system, reproductive system, respiratory system, skeletal system, or combinations thereof), does not have symptoms of the disorder, does not have a genetic mutation associated with the disorder of interest, does not have a family medical history of the disorder of interest, no behavior risk factors of the disorder of interest, or combinations thereof.

In some cases, an amount of a nucleic acid/protein can be elevated relative to a reference amount. For example, an amount of a nucleic acid/protein can be at least 0.2-fold (e.g., at least 0.4-fold, at least 0.6-fold, at least 0.8-fold, at least 1-fold, at least 1.3-fold, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, 30-fold, 40-fold, 50-fold, or more) greater than a reference amount (e.g., any of the exemplary reference amounts described herein or known in the art).

In some cases, an amount of a nucleic acid/protein can be decreased relative to a reference amount. For example, an amount of a nucleic acid/protein can be at least 0.2-fold (e.g., at least 0.4-fold, at least 0.6-fold, at least 0.8-fold, at least 1-fold, at least 1.3-fold, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, 30-fold, 40-fold, 50-fold, or more) less than a reference amount (e.g., any of the exemplary reference amounts described herein or known in the art).

In some cases, an amount of a nucleic acid/protein can be elevated relative to a reference amount. For example, an amount of a nucleic acid can be at least 5% more, at least 10% more, at least 15% more, at least 20% more, at least 25% more, at least 30% more, at least 35% more, at least 40% more, at least 45% more, at least 50% more, at least 55%, at least 60% more, at least 65% more, at least 70% more, at least 75% more, at least 80% more, at least 85% more, at least 90% more, at least 95% elevated (e.g., about a 5% to about a 99% increase, about a 5% increase to about a 80% increase, about a 5% increase to about a 60% increase, about a 5% increase to about a 40% increase, about a 5% increase to about a 20% increase, about a 20% increase to about a 95% increase, about a 20% increase to about a 80% increase, about a 20% increase to about a 60% increase, about a 20% increase to about a 40% increase, about a 40% increase to about a 99% increase, about a 40% increase to about a 80% increase, about a 40% increase to about a 60% increase, about a 60% increase to about a 99% increase, about a 60% increase to about a 80% increase, about a 80% increase to about a 99% increase) as compared to a reference amount (e.g., any of the exemplary reference amounts described herein).

In some cases, an amount of a nucleic acid can be decreased relative to a reference amount. For example, an amount of a nucleic acid/protein can be at least 5% less, at least 10% less, at least 15% less, at least 20% less, at least 25% less, at least 30% less, at least 35% less, at least 40% less, at least 45% less, at least 50% less, at least 55%, at least 60% less, at least 65% less, at least 70% less, at least 75% less, at least 80% less, at least 85% less, at least 90% less, at least 95% decreased (e.g., about a 5% to about a 99% decrease, about a 5% decrease to about a 80% decrease, about a 5% decrease to about a 60% decrease, about a 5% decrease to about a 40% decrease, about a 5% decrease to about a 20% decrease, about a 20% decrease to about a 95% decrease, about a 20% decrease to about a 80% decrease, about a 20% decrease to about a 60% decrease, about a 20% decrease to about a 40% decrease, about a 40% decrease to about a 99% decrease, about a 40% decrease to about a 80% decrease, about a 40% decrease to about a 60% decrease, about a 60% decrease to about a 99% decrease, about a 60% decrease to about a 80% decrease, about a 80% decrease to about a 99% decrease) as compared to a reference amount (e.g., any of the exemplary reference amounts described herein). Other suitable reference amounts and methods of determining the same will be apparent to those skilled in the field.

### III. Methods of Treatment

Disclosed herein are methods of treating a subject having multiple sclerosis (MS). In some instances, the methods include detecting at least one biomarker in cerebrospinal fluid (CSF) in the subject. In some instances, the biomarker is chosen from the proteins listed in Tables 1-3. In some instances, the at least one biomarker is chosen from the proteins listed in Table 3. In some instances, the biomarker is chosen from CXCL13, CXCL10, CD27, NEFL, CCL4, and CCL3. In some instances, the methods include (a) detecting at least one biomarker, at least two biomarkers, at least three biomarkers, at least four biomarkers, at least five biomarkers, at least six biomarkers, or more, in cerebrospinal fluid (CSF) in the subject; and (b) administering a pharmaceutically effective amount of tolebrutinib to the subject. In some instances, the methods include (a) detecting at least one biomarker in a biological sample comprising CSF from the subject; (b) identifying the subject expressing the at least one biomarker in the biological sample as having MS; and (c) administering a pharmaceutically effective amount of tolebrutinib to the subject. In some instances, the methods include detecting at least two biomarkers, at least three biomarkers, at least four biomarkers, at least five biomarkers, at least six biomarkers, or more. In some instances, the methods further include at least one or more additional therapies, such as a pharmaceutically effective amount of an anti-CD20 antibody.

Disclosed herein are methods of treating a subject having multiple sclerosis (MS). In some instances, the methods include (a) detecting CXCL13 in cerebrospinal fluid (CSF) in the subject; and (b) administering a pharmaceutically effective amount of tolebrutinib to the subject. In some instances, the methods include (a) detecting CXCL13 in a biological sample comprising CSF from the subject; (b) identifying the subject expressing CXCL13 in the biological sample as having MS; and (c) administering a pharmaceutically effective amount of tolebrutinib to the subject. In some instances, the methods further include one or more additional therapies, such as an anti-CD20 antibody.

In some instances, the methods include (a) detecting CXCL10 in cerebrospinal fluid (CSF) in the subject; and (b) administering a pharmaceutically effective amount of tolebrutinib to the subject. In some instances, the methods include (a) detecting CXCL10 in a biological sample comprising CSF from the subject; (b) identifying the subject expressing CXCL13 in the biological sample as having MS; and (c) administering a pharmaceutically effective amount of tolebrutinib to the subject. In some instances, the methods further include one or more additional therapies, such as an anti-CD20 antibody.

In some instances, the methods include (a) detecting CD27 in cerebrospinal fluid (CSF) in the subject; and (b) administering a pharmaceutically effective amount of tolebrutinib to the subject. In some instances, the methods include (a) detecting CD27 in a biological sample comprising CSF from the subject; (b) identifying the subject expressing CD27 in the biological sample as having MS; and (c) administering a pharmaceutically effective amount of tolebrutinib to the subject. In some instances, the methods further include one or more additional therapies, such as an anti-CD20 antibody.

In some instances, the methods include (a) detecting NEFL in cerebrospinal fluid (CSF) in the subject; and (b) administering a pharmaceutically effective amount of tolebrutinib to the subject. In some instances, the methods include (a) detecting NEFL in a biological sample comprising CSF from the subject; (b) identifying the subject expressing NEFL in the biological sample as having MS; and (c) administering a pharmaceutically effective amount of tolebrutinib to the subject. In some instances, the methods further include one or more additional therapies, such as an anti-CD20 antibody.

In some instances, the methods include (a) detecting CCL4 in cerebrospinal fluid (CSF) in the subject; and (b) administering a pharmaceutically effective amount of tolebrutinib to the subject. In some instances, the methods include (a) detecting CCL4 in a biological sample comprising CSF from the subject; (b) identifying the subject expressing CCL4 in the biological sample as having MS; and (c) administering a pharmaceutically effective amount of tolebrutinib to the subject. In some instances, the methods further include one or more additional therapies, such as an anti-CD20 antibody.

In some instances, the methods include (a) detecting CCL3 in cerebrospinal fluid (CSF) in the subject; and (b) administering a pharmaceutically effective amount of tolebrutinib to the subject. In some instances, the methods include (a) detecting CCL3 in a biological sample comprising CSF from the subject; (b) identifying the subject expressing CCL3 in the biological sample as having MS; and (c) administering a pharmaceutically effective amount of tolebrutinib to the subject. In some instances, the methods further include one or more additional therapies, such as an anti-CD20 antibody.

In some instances, the methods include (a) detecting CXCL13, CXCL10, CD27, NEFL, CCL4, and/or CCL3 in cerebrospinal fluid (CSF) in the subject; and (b) administering a pharmaceutically effective amount of tolebrutinib to the subject. In some instances, the methods include (a) detecting CXCL13, CXCL10, CD27, NEFL, CCL4, and/or CCL3 in a biological sample comprising CSF from the subject; (b) identifying the subject expressing CXCL13, CXCL10, CD27, NEFL, CCL4, and/or CCL3 in the biological sample as having MS; and (c) administering a pharmaceutically effective amount of tolebrutinib to the subject. In some instances, the methods further include one or more additional therapies, such as an anti-CD20 antibody.

In some instances, the subject has been treated with one or more additional therapies. For instance, a subject can be treated with an anti-CD20 antibody for some time (e.g., weeks, or months) prior to treatment with tolebrutinib. In some instances, it may be beneficial to treat the subject with one or more doses of an anti-CD20 antibody during treatment with tolebrutinib.

In some embodiments, the administration of tolebrutinib reduces the total number of lesions after 12 or 48 weeks of tolebrutinib treatment. In some embodiments, the administration of tolebrutinib maintains—but does not increase—the total number of lesions after 12 or 48 weeks of tolebrutinib treatment. In some instances, the administration of tolebrutinib and one or more additional therapies (such as an anti-CD20 antibody) reduces the total number of lesions after 12 or 48 weeks of treatment. In some instances, the administration of tolebrutinib and one or more additional therapies (such as an anti-CD20 antibody) maintains—but does not increase—the total number of lesions after 12 or 48 weeks of treatment.

In some embodiments, a BTK inhibitor compound, (R)-1-(1-acryloylpiperidin-3-yl)-4-amino-3-(4-phenoxyphenyl)-1H-imidazo[4,5-c]pyridin-2 (3H)-one is administered for treating relapsing multiple sclerosis (RMS) in a subject in need thereof. In some embodiments, the BTK inhibitor compound is a pharmaceutically acceptable salt of (R)-1-(1-acryloylpiperidin-3-yl)-4-amino-3-(4-phenoxyphenyl)-1H-imidazo[4,5-c]pyridin-2 (3H)-one. In some embodiments, a therapeutically effective amount of the BTK inhibitor (e.g., tolebrutinib) is administered. In some embodiments, a dose of 5 to 60 mg of the BTK inhibitor (e.g., tolebrutinib) is administered.

The BTK inhibitor (e.g., tolebrutinib) can be prepared according to the methods and schemes described in, e.g., U.S. Pat. No. 9,688,676 B2 and U.S. Publ. No. US20210244720A1, each of which is incorporated by reference in its entirety.

After detecting at least one biomarker in cerebrospinal fluid (CSF) in the subject (e.g., using any one of the methods described herein), the disclosure provides methods of treating MS in the subject by administering a therapeutically effective amount of a BTK inhibitor (e.g., tolebrutinib). In some instances, the at least one biomarker is chosen from the proteins listed in Tables 1-3. In some instances, the at least one biomarker is chosen from the proteins listed in Table 3. In some instances, the at least one biomarker is chosen from CXCL13, CXCL10, CD27, NEFL, CCL4, and CCL3. After detecting of at least one biomarker, such as CXCL13, CXCL10, CD27, NEFL, CCL4, and/or CCL3, in cerebrospinal fluid (CSF) in the subject (e.g., using any one of the methods described herein), the disclosure provides methods of treating MS in the subject by administering a therapeutically effective amount of a BTK inhibitor (e.g., tolebrutinib). Thus, after detection, provided herein are methods of treating multiple sclerosis comprising administering to a subject in need thereof a therapeutically effective amount of the BTK inhibitor comprising (R)-1-(1-acryloylpiperidin-3-yl)-4-amino-3-(4-phenoxyphenyl)-1H-imidazo[4,5-c]pyridin-2(3H)-one (e.g., tolebrutinib), and/or a pharmaceutically acceptable salt thereof.

In some embodiments the therapeutically effective amount is about 5 to about 60 mg. In some embodiments, a dose of about 5-10 mg, 10-15 mg, 15-20 mg, 20-25 mg, 25-30 mg, 30-35 mg, 35-40 mg, 40-45 mg, 45-50 mg, 50-55 mg, or 55-60 mg is administered. In some embodiments, the dose is 5 mg, 10 mg, 15 mg, 20 mg, 25 mg, 30 mg, 35 mg, 40 mg, 45 mg, 50 mg, 55 mg, or 60 mg. In some embodiments, the dose is 5 mg. In some embodiments, the dose is 15 mg. In some embodiments, the dose is 30 mg. In some embodiments, the dose is 60 mg.

In some embodiments, the dose is administered daily. The daily dose can be delivered as a single dose or split into multiple parts. For example, in some embodiments, the dose is administered once a day (e.g., about every 24 hours). In some embodiments, the dose is administered twice daily. In some embodiments, the dose is subdivided in two parts to be administered twice per day (e.g., about every 12 hours). In some embodiments, the dose is subdivided in three parts to be administered three times per day (e.g., about every 8 hours). In some embodiments, the dose is subdivided in four parts to be administered four times per day (e.g., about every 6 hours).

In some embodiments, the dose is administered orally. In some embodiments, the dose is administered in a form of tablets. In some embodiments, the dose is administered in the form of pills, capsules, semisolids, powders, sustained release formulations, solutions, suspensions, elixirs, aerosols, or any other appropriate compositions.

In some embodiments, the subject is administered the BTK inhibitor (e.g., tolebrutinib) for a period of about 4, 8, 12, 16, 20 weeks, or 48 weeks. In some embodiments, the subject is administered the BTK inhibitor (e.g., tolebrutinib) for a period of about 12 weeks. In some embodiments, the subject is administered the BTK inhibitor (e.g., tolebrutinib) for a period of about 48 weeks. In some embodiments, the dose is once daily.

In some embodiments, the dose is administered with food. In some embodiments, the dose is administered once daily with food. In some embodiments, the dose of 5 mg, 15 mg, 30 mg, or 60 mg is administered with food. In some embodiments, the dose of 5 mg, 15 mg, 30 mg, or 60 mg is administered once daily with food. In some embodiments, the dose of 60 mg is administered once daily with food. In some embodiments, the dose is administered in oral solution or tablets. In some embodiments, the dose is administered in oral solution or tablets with food. In some embodiments, the dose is administered once daily in oral solution or tablets. In some embodiments, the dose is administered once daily in oral solution or tablets with food. In some embodiments, the dose of 60 mg is administered in oral solution or tablets. In some embodiments, the dose of 60 mg is administered in oral solution or tablets with food. In some embodiments, the dose of 60 mg is administered once daily in oral solution or tablets. In some embodiments, the dose of 60 mg is administered once daily in oral solution or tablets with food

Subjects having multiple sclerosis, in some instances, can have conventional T1 and/or T2-weighted (T2w) lesions (e.g., as detected by magnetic resonance (MR) imaging).

In some embodiments, administration of the BTK inhibitor (e.g., tolebrutinib) reduces new active brain lesions. In some embodiments, administration of the BTK inhibitor reduces new active lesions. In some embodiments, administration of the BTK inhibitor (e.g., tolebrutinib) reduces new or enlarging lesions. In some instances, administration of the BTK inhibitor inhibits the formation of new active brain lesions as measured by MRI.

In some embodiments, administration of the BTK inhibitor (e.g., tolebrutinib) reduces the number of new T1 lesions as measured by MRI. In some embodiments, the number of new T1 lesions is less than 1. In some embodiments, the number of new T1 lesions is equal to or less than 0.77, 0.7, 0.6, 0.5, 0.4, 0.3, 0.2, or 0.1. In some embodiments, no new T1 lesions is formed after 12 weeks of BTK inhibitor (e.g., tolebrutinib) treatment.

In some embodiments, administration of the BTK inhibitor (e.g., tolebrutinib) reduces the number of new or enlarging T2 lesions as measured by MRI. In some embodiments, the number of new or enlarging T2 lesions is equal to or less than 2. In some embodiments, the number of new or enlarging T2 lesions is equal to or less than 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1. In some embodiments, no new or enlarging T2 lesion is formed after 12 weeks of BTK inhibitor (e.g., tolebrutinib) treatment.

In some instances, the BTK inhibitor compound is administered as monotherapy.

In some instances, the subject is treated with one or more additional therapies. For instances, the additional therapy can be an anti-CD20 antibody. The “CD20” antigen, or “CD20,” is an about 35-kDa, non-glycosylated phosphoprotein found on the surface of greater than 90% of B cells from peripheral blood or lymphoid organs. CD20 is present on both normal B cells as well as malignant B cells, but is not expressed on stem cells. Other names for CD20 in the literature include “B-lymphocyte-restricted antigen” and “Bp35”. The CD20 antigen is described in Clark et al. Proc. Natl. Acad. Sci. (USA) 82:1766 (1985), for example.

In some embodiments, the anti-CD20 antibody is ocrelizumab.

CDR L1 sequence: (SEQ ID NO: 1):
RASSSVSYMH
CDR L2 sequence (SEQ ID NO: 2):
APSNLAS
CDR L3 sequence (SEQ ID NO: 3):
QQWSFNPPT
CDR H1 sequence (SEQ ID NO: 4):
GYTFTSYNMH
CDR H2 sequence (SEQ ID NO: 5):
AIYPGNGDTSYNQKFKG
CDR H3 sequence (SEQ ID NO: 6):
VVYYSNSYWYFDV

Ocrelizumab comprises the variable light chain sequence:

(SEQ ID NO: 7)
DIQMTQSPSSLSASVGDRVTITCRASSSVSYMHWY
QQKPGKAPKPLIYAPSNLASGVPSRFSGSGSGTDF
TLTISSLQPEDFATYYCQQWSFNPPTFGQGTKVEI
KR;

and the variable heavy chain sequence:

(SEQ ID NO: 8)
EVQLVESGGGLVQPGGSLRLSCAASGYTFTSYNMH
WVRQAPGKGLEWVGAIYPGNGDTSYNQKFKGRFTI
SVDKSKNTLYLQMNSLRAEDTAVYYCARVVYYSNS
YWYFDVWGQGTLVTVSS.

Ocrelizumab comprises the light chain amino acid sequence:

(SEQ ID NO: 9)
DIQMTQSPSSLSASVGDRVTITCRASSSVSYMHWY
QQKPGKAPKPLIYAPSNLASGVPSRFSGSGSGTDF
TLTISSLQPEDFATYYCQQWSFNPPTFGQGTKVEI
KRTVAAPSVFIFPPSDEQLKSGTASVVCLLNNFYP
REAKVQWKVDNALQSGNSQESVTEQDSKDSTYSLS
STLTLSKADYEKHKVYACEVTHQGLSSPVTKSFNR
GEC;

and the heavy chain amino acid sequence:

(SEQ ID NO: 10)
EVQLVESGGGLVQPGGSLRLSCAASGYTFTSYNMH
WVRQAPGKGLEWVGAIYPGNGDTSYNQKFKGRFTI
SVDKSKNTLYLQMNSLRAEDTAVYYCARVVYYSNS
YWYFDVWGQGTLVTVSSASTKGPSVFPLAPSSKST
SGGTAALGCLVKDYFPEPVTVSWNSGALTSGVHTF
PAVLQSSGLYSLSSVVTVPSSSLGTQTYICNVNHK
PSNTKVDKKVEPKSCDKTHTCPPCPAPELLGGPSV
FLFPPKPKDTLMISRTPEVTCVVVDVSHEDPEVKF
NWYVDGVEVHNAKTKPREEQYNSTYRVVSVLTVLH
QDWLNGKEYKCKVSNKALPAPIEKTISKAKGQPRE
PQVYTLPPSREEMTKNQVSLTCLVKGFYPSDIAVE
WESNGQPENNYKTTPPVLDSDGSFFLYSKLTVDKS
RWQQGNVFSCSVMHEALHNHYTQKSLSLSPGK

or the heavy chain amino acid sequence:

(SEQ ID NO: 11)
EVQLVESGGGLVQPGGSLRLSCAASGYTFTSYNMH
WVRQAPGKGLEWVGAIYPGNGDTSYNQKFKGRFTI
SVDKSKNTLYLQMNSLRAEDTAVYYCARVVYYSNS
YWYFDVWGQGTLVTVSSASTKGPSVFPLAPSSKST
SGGTAALGCLVKDYFPEPVTVSWNSGALTSGVHTF
PAVLQSSGLYSLSSVVTVPSSSLGTQTYICNVNHK
PSNTKVDKKVEPKSCDKTHTCPPCPAPELLGGPSV
FLFPPKPKDTLMISRTPEVTCVVVDVSHEDPEVKF
NWYVDGVEVHNAKTKPREEQYNSTYRVVSVLTVLH
QDWLNGKEYKCKVSNKALPAPIEKTISKAKGQPRE
PQVYTLPPSREEMTKNQVSLTCLVKGFYPSDIAVE
WESNGQPENNYKTTPPVLDSDGSFFLYSKLTVDKS
RWQQGNVFSCSVMHEALHNHYTQKSLSLSPG.

In some instances, the anti-CD20 antibody is humanized. In some instances, the anti-CD20 is included in a pharmaceutical formulation or composition. Anti-CD20 antibodies, compositions, and methods regarding the same are described in US 2022/0064320 A1, which is incorporated by reference in its entirety.

In some embodiments, the amino acid K at C-terminus of the heavy chain is removed.

The term “ocrelizumab” (CAS Registration No. 637334-45-3) herein refers to the genetically engineered humanized monoclonal antibody directed against the CD20 antigen and comprising (a) a light chain comprising the amino acid sequence of SEQ ID NO: 9 and (b) a heavy chain comprising the amino acid sequence of SEQ ID NO: 11, including fragments thereof that retain the ability to bind CD20. Ocrelizumab is available from Genentech.

In some embodiments, the anti-CD20 antibody is rituximab (CAS Registry No. 174722-31-7). Rituximab refers to the genetically engineered humanized monoclonal antibody directed against the CD20 antigen and comprising (a) a light chain comprising the amino acid sequence of SEQ ID NO: 12 and (b) a heavy chain comprising the amino acid sequence of SEQ ID NO: 13, including fragments thereof that retain the ability to bind CD20.

Rituximab comprises the light chain amino acid sequence:

(SEQ ID NO: 12)
QIVLSQSPAILSASPGEKVTMTCRASSSVSYIHWFQQKPG
SSPKPWIYATSNLASGVPVRFSGSGSGTSYSLTISRVEAE
DAATYYCQQWTSNPPTFGGGTKLEIKRTVAAPSVFIFPP
SDEQLKSGTASVVCLLNNFYPREAKVQWKVDNALQSG
NSQESVTEQDSKDSTYSLSSTLTLSKADYEKHKVYACEV
THQGLSSPVTKSFNRGEC;

and the heavy chain amino acid sequence:

(SEQ ID NO: 13)
QVQLQQPGAELVKPGASVKMSCKASGYTFTSYNMHWV
KQTPGRGLEWIGAIYPGNGDTSYNQKFKGKATLTADKS
SSTAYMQLSSLTSEDSAVYYCARSTYYGGDWYFNVWG
AGTTVTVSAASTKGPSVFPLAPSSKSTSGGTAALGCLVK
DYFPEPVTVSWNSGALTSGVHTFPAVLQSSGLYSLSSVV
TVPSSSLGTQTYICNVNHKPSNTKVDKKAEPKSCDKTHT
CPPCPAPELLGGPSVFLFPPKPKDTLMISRTPEVTCVVVD
VSHEDPEVKFNWYVDGVEVHNAKTKPREEQYNSTYRV
VSVLTVLHQDWLNGKEYKCKVSNKALPAPIEKTISKAK
GQPREPQVYTLPPSRDELTKNQVSLTCLVKGFYPSDIAV
EWESNGQPENNYKTTPPVLDSDGSFFLYSKLTVDKSRW
QQGNVFSCSVMHEALHNHYTQKSLSLSPGK.

In certain embodiments, provided is a method of treating multiple sclerosis in a patient, comprising administering an effective amount of an anti-CD20 antibody to the patient, wherein the anti-CD20 antibody comprises a VH domain comprising the amino acid set forth in SEQ ID NO: 8, a VL domain comprising the amino acid sequence set forth in SEQ ID NO: 7. In some instances, the anti-CD20 antibody comprises a human IgG1 constant region. In some embodiments, the anti-CD20 antibody comprises a light chain comprising the amino acid sequence of SEQ ID NO: 9 and a heavy chain comprising the amino acid sequence of SEQ ID NO: 11. In some embodiments, the anti-CD20 antibody is ocrelizumab (CAS Registry No. 637334-45-3).

In certain embodiments, provided is a method of treating multiple sclerosis in a patient, comprising administering an effective amount of an anti-CD20 antibody to the patient, wherein the anti-CD20 antibody comprises a light chain comprising the amino acid sequence of SEQ ID NO: 12 and a heavy chain comprising the amino acid sequence of SEQ ID NO: 13. In some embodiments, the anti-CD20 antibody is rituximab (CAS Registry No. 174722-31-7).

In some embodiments, the initial anti-CD20 antibody dose comprises one or more intravenous infusion (e.g., intravenous (IV) infusion) of an anti-CD20 antibody. The one or more IV infusions can be administered over a period of time (e.g., for at least six months). In some embodiments the subject is administered the IV infusion at doses that are about a week to about three weeks (e.g., about 1 week, about 2 weeks, or about 3 weeks) apart.

In one embodiment, the patient has never been previously treated with drug(s), such as immunosuppressive agent(s), to treat the multiple sclerosis and/or has never been previously treated with an antibody to a B-cell surface marker (e.g. never previously treated with a CD20 antibody).

In certain embodiments, the patient is premedicated prior to infusion with the anti-CD20 antibody. In certain embodiments, the patient is premedicated with methylprednisolone (or an equivalent) approximately 30 minutes prior to each infusion of anti-CD20 antibody. In certain embodiments, the patient is premedicated with 100 mg IV methylprednisolone (or an equivalent) approximately 30 minutes prior to each infusion of anti-CD20 antibody. In certain embodiments, the patient is additionally (or alternatively) premedicated with an antihistaminic drug (e.g. diphenhydramine) approximately 30-60 minutes before each infusion of anti-CD20 antibody. In certain embodiments, the patient is additionally (or alternatively) premedicated with an antipyretic (e.g. acetaminophen/paracetamol).

While the CD20 antibody may be the only drug administered to the patient to treat the multiple sclerosis, one may optionally administer a second medicament, e.g., a second multiple sclerosis disease modifying agent (DMT), such as a cytotoxic agent, chemotherapeutic agent, immunosuppressive agent, cytokine, cytokine antagonist or antibody, growth factor, hormone, integrin, integrin antagonist or antibody (e.g. an LFA-1 antibody, or an alpha 4 integrin antibody such as natalizumab (TYSABRI®) available from Biogen Idec/Elan Pharmaceuticals, Inc.) etc., with the antibody that binds a B cell surface marker (e.g. with the CD20 antibody).

### EXAMPLES

### Example 1. Evaluating Large Scale Proteomic Changes in Cerebrospinal Fluid of Multiple Sclerosis Patients

### Example 1A. Background

Molecular biomarkers are needed to measure multiple sclerosis (MS) disease activity and to evaluate therapeutic efficacy. Often times, samples taken from a subject (e.g., blood samples) are not correlative to the bioactivity occurring in the brain of the subject. Thus, herein, proteins from the cerebrospinal fluid (CSF) were examined in order to examine biomarkers into certain pathophysiologies such as neuroinflammation. Such biomarkers, it was hypothesized, might serve as prognostic indicators of disease course and could help provide evidence of treatment response following therapy.

In the present study, a platform technology (Olink® Proteomics) was used. This test utilizes a high throughput, multiplex immunoassay technology that enables the measurement of over 1000 proteins from small sample volumes. See e.g., Assarsson E, et al. PLoS One 2014; 9(4): e95192; and Cui M, et al. Lab Invest 2022, each of which is incorporated by reference in its entirety. Two sets of subjects were analyzed. First, Olink proteomics technology was used to profile the baseline CSF proteome of untreated MS patients. Second, alterations to the MS CSF proteome upon therapeutic intervention with either a B cell depleting agent alone or after transitioning to tolebrutinib, a brain penetrant Bruton's tyrosine kinase (BTK) inhibitor, was evaluated.

### Example 1B. Baseline CSF Proteome of Untreated MS Patients

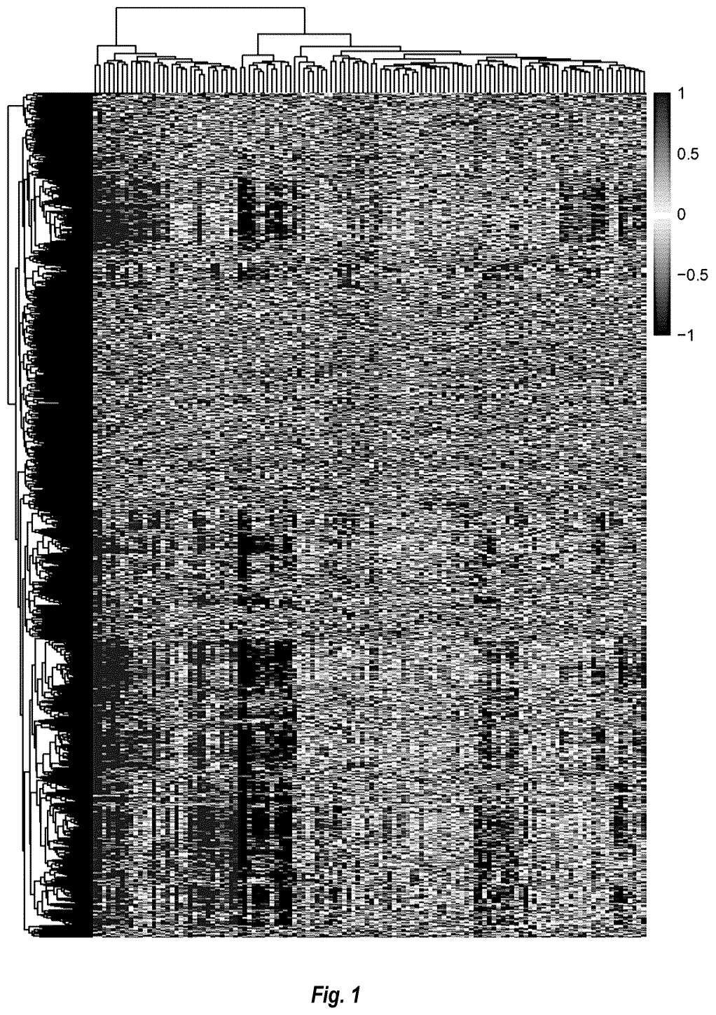

To establish a baseline of proteomic expression, an Olink proteomics assay was performed on the CSF of multiple groups of subjects, including healthy volunteers (HV), HTLV 1 associated myelopathy/tropical spastic paraparesis (HAM/TSP) patients, radiologically isolated syndrome (RIS) patients, clinically isolated syndrome (CIS) patients, and multiple sclerosis (MS) patients, including patients with relapsing remitting MS (RRMS), secondary progressive MS (SPMS), and primary progressive MS (PPMS). CSF samples were collected from 31 healthy volunteers and 71 treatment-naïve subjects with MS. The CSF proteome was examined using the Cardiometabolic, Inflammation, Neurology, and Oncology Olink Explore 384 panels (1463 analytes total). A heatmap is shown in FIG. 1.

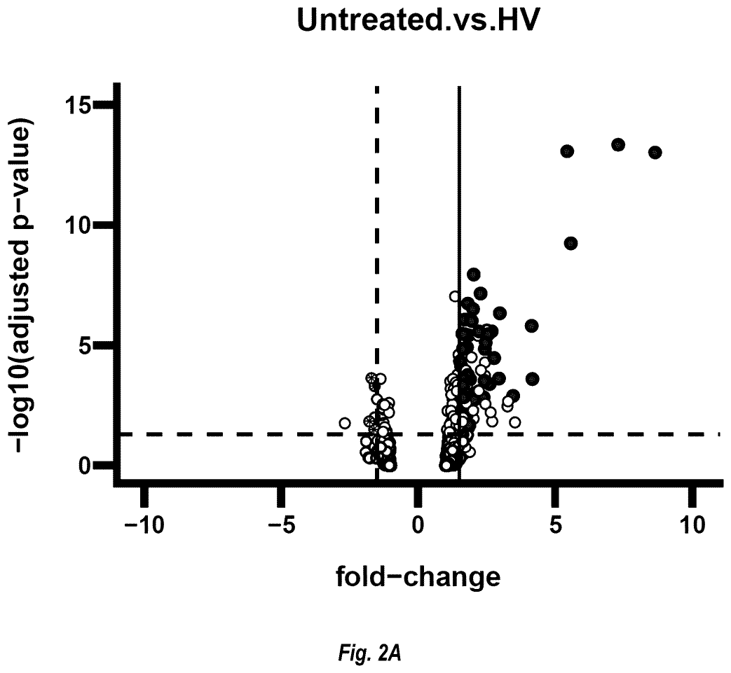

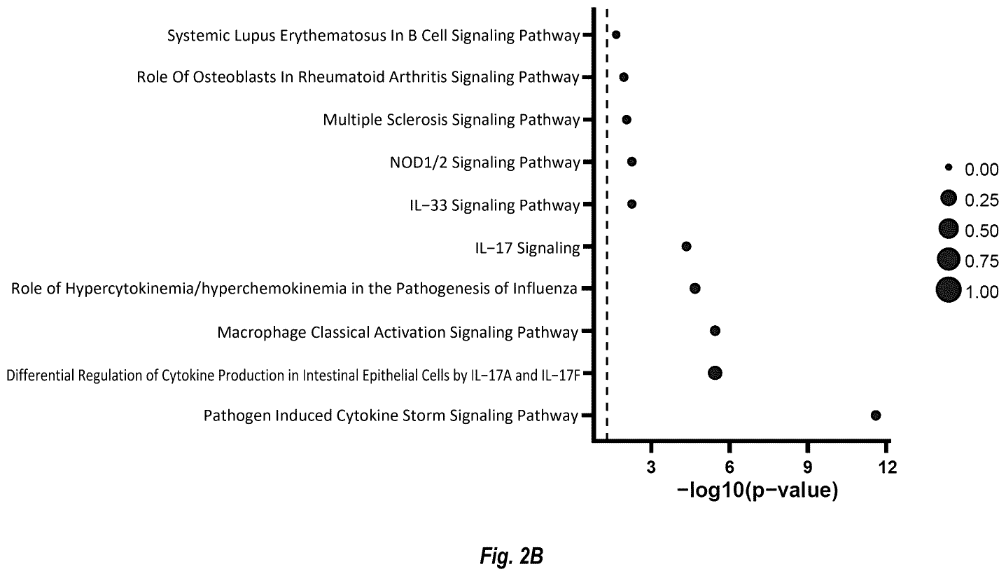

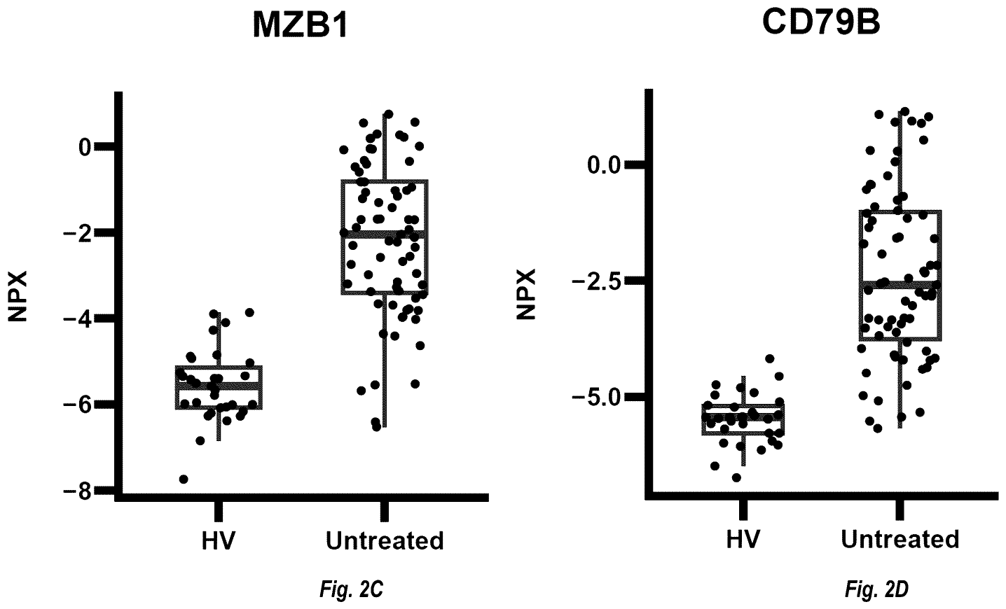

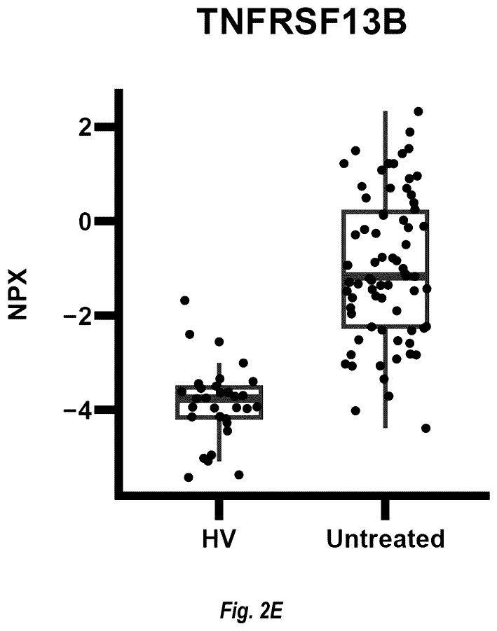

Pathway analysis was performed using Ingenuity Pathway Analysis. The data was sorted into a comparison of untreated MS subjects with healthy volunteers (FIG. 2A). Compared to healthy volunteers, the untreated MS patients showed differential abundance. As seen in Table 1, certain proteins were either underexpressed or overexpressed in untreated MS subjects compared to healthy volunteers.

TABLE 1
Proteins with decreased and increased abundance in untreated
MS subjects compared to healthy volunteers

| | P-value | Fold-change |
|---|---|---|
| Protein | (Untreated. vs. HV) | (Untreated. vs. HV) |
| NAMPT | 0.01455 | −1.78787 |
| SORD | 0.000234 | −1.70954 |
| CA2 | 0.031131 | −1.60579 |
| ANXA10 | 0.000496 | −1.59266 |
| CD22 | 0.010203 | 1.510682 |
| LAMP3 | 4.63E−05 | 1.522643 |
| CR2 | 6.83E−05 | 1.539462 |
| CRHBP | 0.011974 | 1.553993 |
| IL5RA | 7.47E−05 | 1.564972 |
| CLUL1 | 0.020819 | 1.57188 |
| STC1 | 0.00877 | 1.576267 |
| KRT18 | 0.007674 | 1.591121 |
| CD160 | 0.002116 | 1.59248 |
| ADA2 | 3.35E−06 | 1.608932 |
| SCG2 | 0.049844 | 1.609407 |
| MERTK | 0.008241 | 1.617093 |
| NTproBNP | 0.013547 | 1.6298 |
| TNFSF14 | 1.41E−05 | 1.632129 |
| FCRL2 | 0.002331 | 1.649645 |
| GZMH | 0.0002 | 1.656179 |
| CD8A | 0.009781 | 1.659076 |
| MSLN | 0.000435 | 1.659101 |
| CDH3 | 0.001596 | 1.661506 |
| NUCB2 | 0.001438 | 1.666178 |
| SLAMF7 | 0.000264 | 1.666521 |
| CLEC4C | 3.44E−06 | 1.675884 |
| CTRC | 0.003858 | 1.679733 |
| ICAM3 | 8.72E−07 | 1.691863 |
| RTBDN | 0.03664 | 1.700035 |
| CCL18 | 0.001382 | 1.728364 |
| PDCD5 | 0.008273 | 1.764349 |
| CST7 | 1.22E−05 | 1.776126 |
| CLEC4D | 0.001596 | 1.798385 |
| CD6 | 1.83E−07 | 1.806944 |
| CCL4 | 0.000169 | 1.807117 |
| LTA | 3.87E−06 | 1.836741 |
| CCL8 | 0.020137 | 1.870584 |
| FCRL1 | 0.000261 | 1.890455 |
| CCL11 | 0.002287 | 1.896543 |
| CXCL11 | 9.84E−07 | 1.955115 |
| CD5 | 3.17E−07 | 2.000341 |
| CD48 | 1.13E−08 | 2.025106 |
| TNFRSF6B | 0.001794 | 2.042263 |
| ANGPTL2 | 0.000992 | 2.050261 |
| FCRL5 | 2.69E−06 | 2.222341 |
| CCL3 | 6.94E−08 | 2.276603 |
| CCL17 | 0.001442 | 2.387059 |
| CCL19 | 0.0003 | 2.420397 |
| MMP7 | 1.43E−05 | 2.431335 |
| CD38 | 7.79E−06 | 2.461328 |
| CXCL13 | 3.42E−06 | 2.562375 |
| CXCL5 | 0.000405 | 2.606513 |
| MFAP5 | 2.65E−06 | 2.680125 |
| CXCL1 | 3.40E−05 | 2.769349 |
| TDGF1 | 0.00024 | 2.946666 |
| PRL | 4.72E−07 | 2.977637 |
| GBP2 | 0.001257 | 3.463697 |
| CXCL10 | 1.58E−06 | 4.140119 |
| IL10 | 0.000252 | 4.166611 |
| CD27 | 8.67E−14 | 5.436364 |
| NEFL | 5.76E−10 | 5.572494 |
| TNFRSF13B | 4.59E−14 | 7.300288 |
| CD79B | 9.69E−14 | 8.648081 |
| MZB1 | 9.00E−14 | 10.50348 |

As seen in Table 1, Olink analysis detected 64 proteins that had altered levels in the CSF in subjects with untreated MS (4 proteins showed decreased abundance in subjects with untreated MS and 60 proteins showed increased abundance in subjects with untreated MS).

The Olink data showed that MS patients displayed unique protein and pathways compared to healthy volunteers. FIG. 2B. The pathway analysis in FIG. 2B indicated increased markers of macrophage, B- and T-cell activation in MS CSF. As a particular example, MZB1, CD79B, and TNFRSF13B which showed increase from the Olink Proteomic data, are shown as individual biomarkers, demonstrating the capability to examine single proteins using these methods. See FIG. 2C-E. Taken together, these data demonstrate the ability to detect proteins in CSF and show that MS subjects and healthy individuals have distinguishing global proteome expression in CSF.

### Example 1C. Treatment Comprising Intervention with Either a B Cell Depleting Agent Alone or after Transitioning to Tolebrutinib

Next, it was examined whether treatment with different therapies for patients having MS would result in differential proteome expression. A group of subjects with MS were treated either with an anti-CD20 antibody alone or with an anti-CD20 antibody and then transitioning to tolebrutinib, and their proteome was examined using similar techniques described in Example 1B. More specifically, the cohort of MS patients in this example included untreated patients, patients treated with a B cell depleting agent for at least six months, and patients 12 weeks and 48 weeks after transitioning from a B cell depletion therapy to tolebrutinib in a clinical trial (NCT04742400).

Several participants (age [mean±standard deviation]: 48.2±7.9 years; sex: 2 female) who had been treated with ocrelizumab for >6 months (median: 3.2 years; range: 1.8-4.0 years) were included. Participants had no signs of acute focal inflammation based on magnetic resonance imaging. CSF was collected before baseline (within 6 months since last ocrelizumab infusion), and at 12 and 48 weeks after transitioning to tolebrutinib 60 mg.

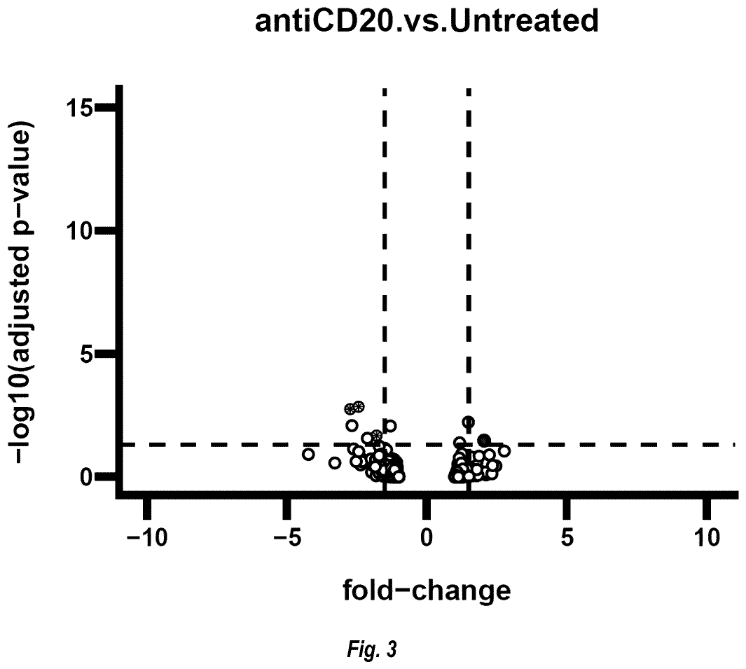

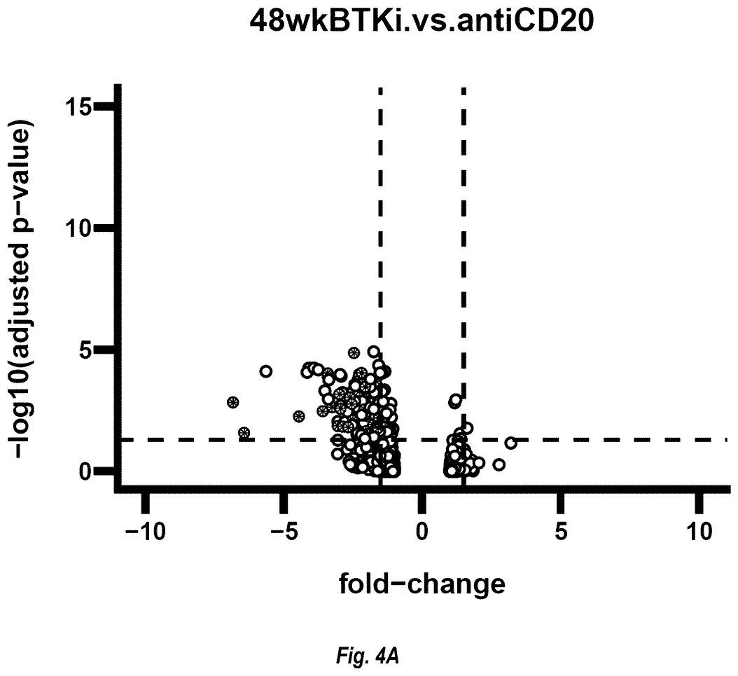

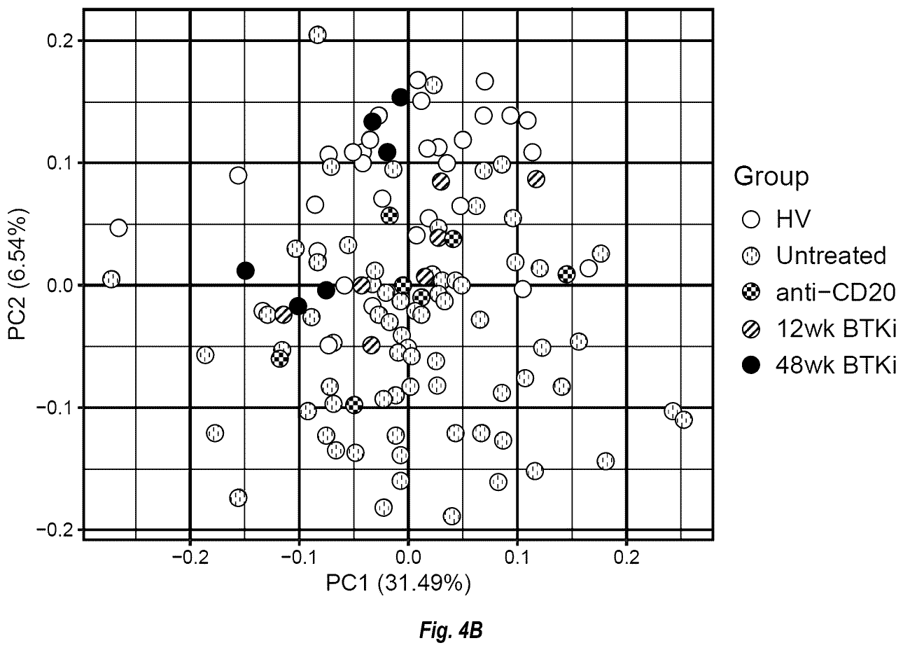

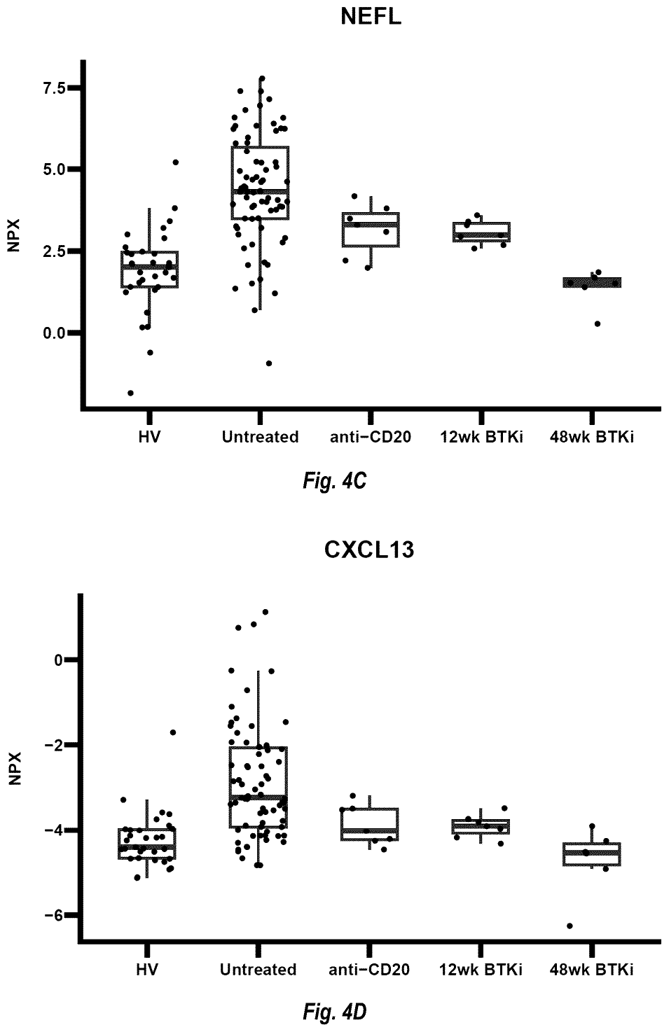

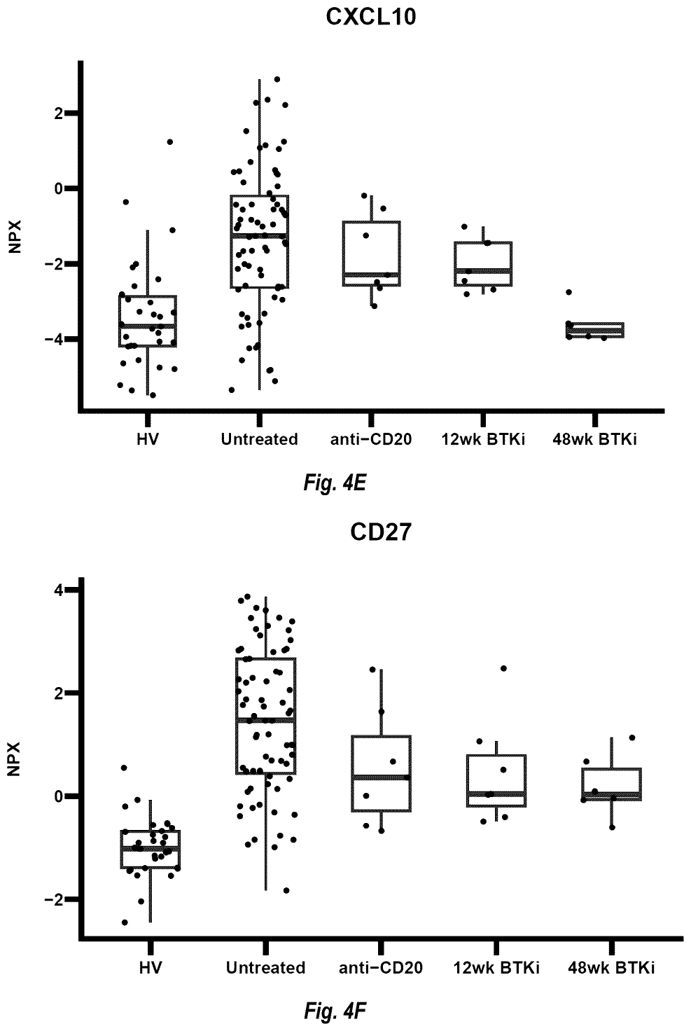

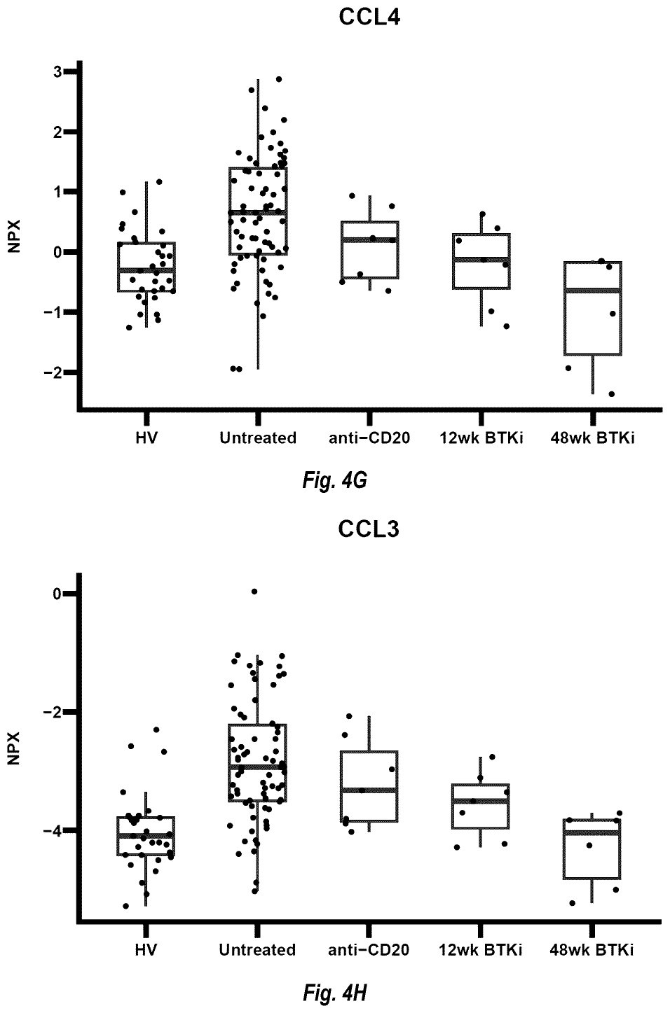

The data were sorted into two sets of comparisons. The comparisons include (1) subjects treated with an anti-CD20 antibody compared to untreated MS subjects (FIG. 3) and (2) subjects treated with tolebrutinib after transitioning from an anti-CD20 antibody compared to subjects treated with an anti-CD20 antibody (FIG. 4A). Compared to untreated MS patients, each group showed differential abundance. As seen in Table 2, certain proteins were either underexpressed or overexpressed in subjects treated with an anti-CD20 antibody compared to untreated MS subjects.

TABLE 2
Proteins with decreased and increased abundance in subjects treated
with anti-CD20 antibody compared to untreated MS subjects

| | P-value (antiCD20. | Fold-change (antiCD20. |
|---|---|---|
| Protein | vs. Untreated) | vs. Untreated) |
| FCRL1 | 0.001792 | −2.74424 |
| CD22 | 0.001429 | −2.44199 |
| CD6 | 0.02118 | −1.80016 |
| SORD | 0.033878 | 2.045093 |

As a particular example, FCRL1 showed nearly a three-fold decrease from the Olink Proteomic data. See Table 2.

Similar to the anti-CD20 results, data was generated in subjects treated with tolebrutinib after transitioning from an anti-CD20 antibody compared to subjects treated with an anti-CD20 antibody. As shown in FIG. 4A, MS subjects treated with tolebrutinib after transitioning from an anti-CD20 antibody exhibited differential protein abundance compared to MS subjects treated with an anti-CD20 antibody.

TABLE 3
Proteins with decreased abundance in subjects treated with
tolebrutinib after transitioning from anti-CD20 antibody
compared to subjects treated with anti-CD20 antibody

| | P-value | Fold-Change |
|---|---|---|
| Protein | (48wkBTKi. vs. antiCD20) | (48wkBTKi. vs. antiCD20) |
| IL11 | 0.001458 | −6.84056 |
| ATXN10 | 0.025762 | −6.43739 |
| TDGF1 | 0.005498 | −4.45691 |
| CXCL10 | 0.003349 | −3.59833 |
| NEFL | 0.000122 | −3.41978 |
| PRL | 9.43E−05 | −3.41578 |
| ANGPTL2 | 0.002287 | −3.26564 |
| CXCL1 | 0.013439 | −3.05418 |
| TNFRSF6B | 0.000668 | −2.99928 |
| SCG2 | 0.002651 | −2.95885 |
| CTRC | 0.01418 | −2.86871 |
| CCL8 | 0.014957 | −2.68004 |
| RTBDN | 0.000905 | −2.62544 |
| NUCB2 | 0.001685 | −2.55115 |
| MFAP5 | 0.00113 | −2.53555 |
| PDCD5 | 0.007766 | −2.51135 |
| NPDC1 | 1.37E−05 | −2.47063 |
| WFIKKN2 | 0.000675 | −2.43247 |
| NTproBNP | 0.000149 | −2.31364 |
| CDH3 | 0.000548 | −2.29318 |
| SCG3 | 0.000103 | −2.2848 |
| CHRDL1 | 0.004695 | −2.23649 |
| CD276 | 0.000256 | −2.17273 |
| CCL3 | 0.001169 | −2.14175 |
| NPTX1 | 0.000396 | −2.10252 |
| CDNF | 0.00282 | −2.09997 |
| CCL4 | 0.00128 | −2.09217 |
| MSLN | 0.000343 | −2.06843 |
| TNFRSF13B | 0.012991 | −2.06282 |
| DRAXIN | 0.000556 | −2.04677 |
| CD38 | 0.022306 | −2.03535 |
| CLUL1 | 0.000719 | −2.02055 |
| WIF1 | 0.029215 | −1.99364 |
| MZB1 | 0.000718 | −1.96761 |
| CCL11 | 0.019116 | −1.9597 |
| SPRY2 | 0.00097 | −1.90678 |
| MATN3 | 0.005967 | −1.88902 |
| STC1 | 0.02653 | −1.86331 |
| NPY | 0.00159 | −1.79742 |
| TIMP4 | 0.012009 | −1.77558 |
| CD8A | 0.025998 | −1.75029 |
| LPL | 0.002373 | −1.74417 |
| IL5RA | 0.029362 | −1.71608 |
| CLEC4C | 0.026882 | −1.71398 |
| HS3ST3B1 | 0.004963 | −1.70758 |
| CD4 | 0.006128 | −1.70561 |
| HYOU1 | 0.0002 | −1.67604 |
| ANGPT1 | 0.040518 | −1.65786 |
| KRT18 | 0.013782 | −1.6528 |
| NUB1 | 0.005556 | −1.62411 |
| CCN4 | 0.016686 | −1.61147 |
| CXCL8 | 0.028158 | −1.60473 |
| CXCL8 | 0.028158 | −1.60473 |
| CXCL8 | 0.028158 | −1.60473 |
| CXCL8 | 0.028158 | −1.60473 |
| CD302 | 0.006348 | −1.5791 |
| LTA | 0.007732 | −1.55238 |
| KAZALD1 | 0.020183 | −1.54769 |
| CGREF1 | 0.000102 | −1.53471 |
| ROBO1 | 0.031755 | −1.51107 |

As seen in Table 3, 60 proteins demonstrated decreased abundance 48 weeks post-transition to tolebrutinib and 0 proteins showed increased abundance 48 weeks post-transition to tolebrutinib. 30 proteins with increased abundance in subjects with MS (relative to HV) were reversed in samples taken 48 weeks post-transitioning to tolebrutinib relative to their anti-CD20 baseline levels. The CSF proteome of people with MS was altered 48 weeks after transitioning to tolebrutinib from a B-cell depleting therapy, with 30 disease-associated proteins (47%) reverting towards levels observed in healthy volunteers.

Principal component analysis (PCA) of Olink Proteomics dataset colored by treatment is shown in FIG. 4B. Six example biomarkers, NEFL (FIG. 4C), CXCL13 (FIG. 4D), CXCL10 (FIG. 4E), CD27 (FIG. 4F), CCL4 (FIG. 4G), and CCL3 (FIG. 4H), were differentially expressed, demonstrating the capability to examine single proteins using these methods. The biomarkers are examples of disease-reversed proteins 48 weeks after transitioning from anti-CD20 therapy to tolebrutinib.

### Example 2. Identification of CXCL13 in CSF as a Biomarker of Multiple Sclerosis

Using the same dataset that was generated in Example 1C, a cohort of subjects was examined for detection of proteome changes. In this cohort, subjects were given ocrelizumab for up to six months. Then, a subset of patients was switched from ocrelizumab treatment to treatment with tolebrutinib for up to forty-eight weeks. After, CSF was extracted, and proteome analysis was performed using the Olink Proteome assay.

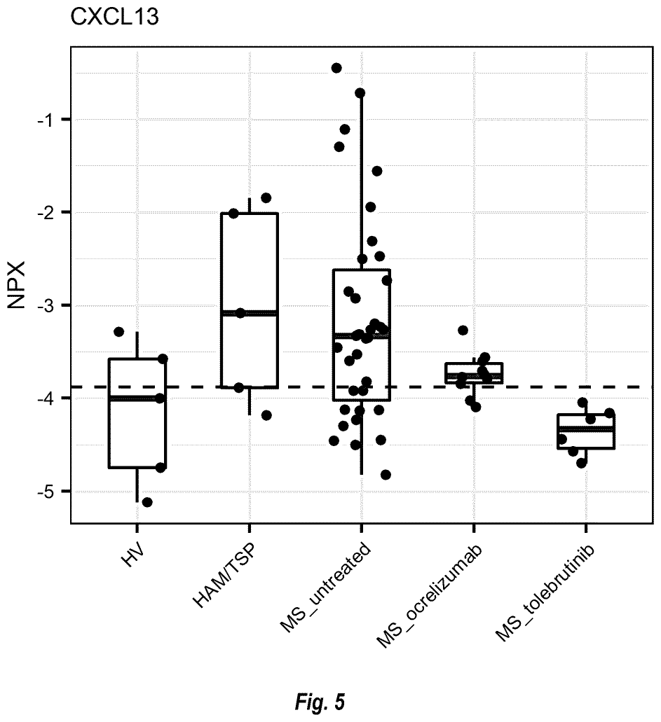

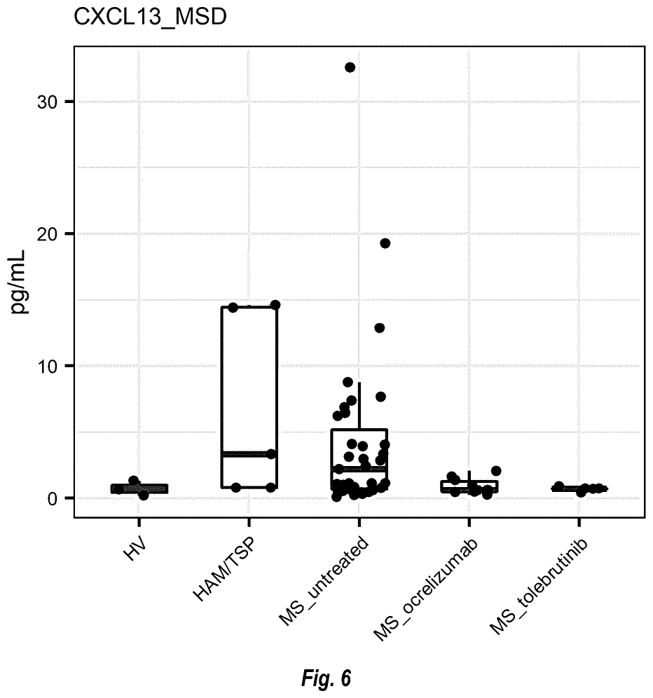

In the assay, it was identified that CXCL13 was downregulated in subjects who switched treatment from ocrelizumab to tolebrutinib. Compared to subjects having HTLV-1 associated myelopathy/tropical spastic paraparesis or undergoing no treatment, subjects treated with a twelve week and forty-eight week course of tolebrutinib exhibited a decrease in CXCL13 in CSF. Similarly, this decrease was seen when comparing subjects who continued treatment with the anti-CD20 antibody (ocrelizumab) with subjects treated with a twelve week and forty-eight week course of tolebrutinib. FIGS. 4D, 5, and 6. These data suggest that treatment with tolebrutinib, particularly after a course of treatment with ocrelizumab, can lead (o decreased protein levels of CXCL13, which is associated with more favorable clinical outcomes, including active lesions. FIG. 6 is a separate method for measuring protein levels (MSD—meso scale discovery), so we see these shifts in CXCL13 in multiple modalities.

### Example 3. Identification of CXCL10 in CSF as a Biomarker of Multiple Sclerosis

Using the same dataset that was generated in Example 1C, a cohort of subjects was examined for detection of proteome changes. In this cohort, subjects were given ocrelizumab for up to six months. Then, a subset of patients was switched from ocrelizumab treatment to treatment with tolebrutinib for up to forty-eight weeks. After, CSF was extracted, and proteome analysis was performed using the Olink Proteome assay.

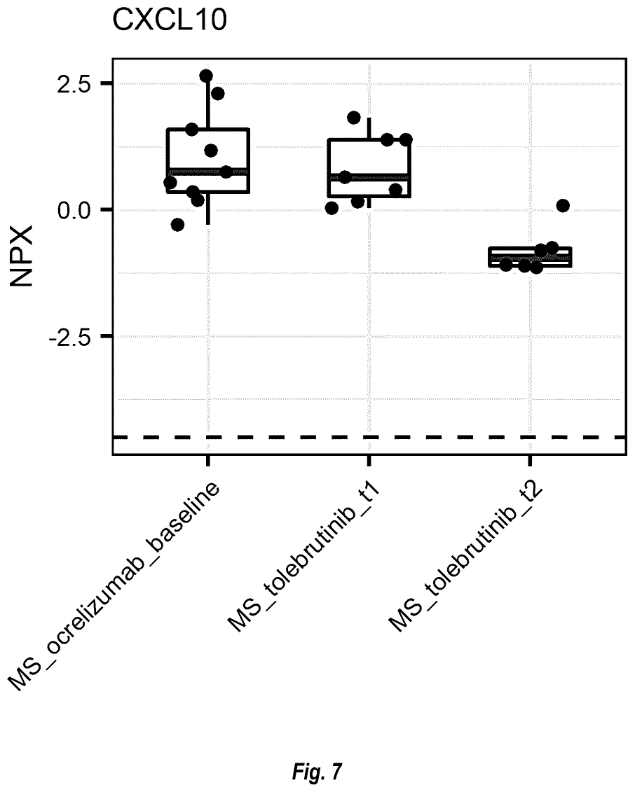

FIGS. 4E and 7 herein shows that CXCL10 was measured in cerebrospinal fluid (CSF) of patients with multiple sclerosis via Olink proteomics technology. In FIG. 4E, the cohort of patients with MS included patients treated with ocrelizumab for at least 6 months (anti-CD20), patients 12 weeks after transitioning from a B-cell depletion therapy to tolebrutinib (12wk BTKi), and patients 48 weeks after transitioning from a B-cell depletion therapy to tolebrutinib (48wk BTKi). In FIG. 7, the cohort of patients with MS included patients treated with ocrelizumab for at least 6 months (MS_ocrelizumab_baseline), patients 12 weeks after transitioning from a B-cell depletion therapy to tolebrutinib (MS_tolebrutinib_t1), and patients 48 weeks after transitioning from a B-cell depletion therapy to tolebrutinib (MS_tolebrutinib_t2) in a Phase 2 clinical trial (NCT04742400).

In the assay, it was identified that CXCL10 was downregulated in subjects who switched treatment from ocrelizumab to tolebrutinib. These data suggest that treatment with tolebrutinib, particularly after a course of treatment with ocrelizumab, can lead to decreased protein levels of CXCL10, which is associated with more favorable clinical outcomes, including active lesions.

### OTHER EMBODIMENTS

While the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

What is claimed is:

1. A method of treating a subject having MS, the method comprising:
(a) detecting CXCL13 in a biological sample comprising CSF from the subject;
(b) identifying the subject expressing CXCL13 in the biological sample as having MS; and
(c) administering a pharmaceutically effective amount of tolebrutinib to the subject.

2. A method of identifying a patient as being suitable for participating in a clinical trial for MS, the method comprising:
(a) detecting CXCL13 in a biological sample comprising CSF from the subject; and
(b) identifying the subject expressing CXCL13 in the biological sample, thereby identifying a patient as being suitable for participating in a clinical trial for MS.

3. A method of monitoring progression of MS in a subject over time, the method comprising:
(a) detecting CXCL13 in a first biological sample obtained from a subject at a first time point;
(b) detecting CXCL13 in a second biological sample obtained from the subject at a second time point; and
(c) identifying:
(i) a subject having increased CXCL13 at the second time point, as compared to CXCL13 at the first time point, as having progressing MS, or
(ii) a subject having about the same or a decreased CXCL13 at the second time point, as compared to CXCL13 at the first time point, as having static or regressing MS.

4. A method of assessing the efficacy of a treatment of a pharmaceutically effective amount of tolebrutinib in a subject having MS, the method comprising:
(a) detecting (i) CXCL13 in a first biological sample comprising CSF obtained from the subject at a first time point and (ii) CXCL13 in a second biological sample comprising CSF obtained from the subject at a second time point, wherein the subject is administered one or more doses of the treatment between the first and second time points; and
(b) determining a correlation between efficacy of the treatment and CXCL13 in the second biological sample as compared to CXCL13 in a sample obtained from an untreated patient, wherein the CXCL13 in the second biological sample is about the same or decreased as compared to the abundance in the sample from the untreated patient, thereby indicating that the treatment is effective for MS in the subject.

5. A method of treating a subject having MS, the method comprising:
(a) detecting CXCL10 in a biological sample comprising CSF from the subject;
(b) identifying the subject expressing CXCL10 in the biological sample as having MS; and
(c) administering a pharmaceutically effective amount of tolebrutinib to the subject.

6. A method of identifying a patient as being suitable for participating in a clinical trial for MS, the method comprising:
(a) detecting CXCL10 in a biological sample comprising CSF from the subject; and
(b) identifying the subject expressing CXCL10 in the biological sample, thereby identifying a patient as being suitable for participating in a clinical trial for MS.

7. A method of monitoring progression of MS in a subject over time, the method comprising:
(a) detecting CXCL10 in a first biological sample obtained from a subject at a first time point;
(b) detecting CXCL10 in a second biological sample obtained from the subject at a second time point; and
(c) identifying:
(i) a subject having increased CXCL10 at the second time point, as compared to CXCL10 at the first time point, as having progressing MS, or
(ii) a subject having about the same or a decreased CXCL10 at the second time point, as compared to CXCL10 at the first time point, as having static or regressing MS.

8. A method of assessing the efficacy of a treatment of a pharmaceutically effective amount of tolebrutinib in a subject having MS, the method comprising:
(a) detecting (i) CXCL10 in a first biological sample comprising CSF obtained from the subject at a first time point and (ii) CXCL10 in a second biological sample comprising CSF obtained from the subject at a second time point, wherein the subject is administered one or more doses of the treatment between the first and second time points; and
(b) determining a correlation between efficacy of the treatment and CXCL10 in the second biological sample as compared to CXCL10 in a sample obtained from an untreated patient, wherein the CXCL10 in the second biological sample is about the same or decreased as compared to the abundance in the sample from the untreated patient, thereby indicating that the treatment is effective for MS in the subject.

9. A method of treating a subject having multiple sclerosis (MS), the method comprising:
(a) detecting at least one biomarker in cerebrospinal fluid (CSF) in the subject; and
(b) administering a pharmaceutically effective amount of tolebrutinib to the subject, wherein the at least one biomarker is chosen from CXCL13, CXCL10, CD27, NEFL, CCL4, and CCL3.

10. A method of treating a subject having MS, the method comprising:
(a) detecting at least one biomarker in a biological sample comprising CSF from the subject;
(b) identifying the subject expressing the at least one biomarker in the biological sample as having MS; and
(c) administering a pharmaceutically effective amount of tolebrutinib to the subject;
wherein the at least one biomarker is chosen from CXCL13, CXCL10, CD27, NEFL, CCL4, and CCL3.

11. A method of identifying a patient as being suitable for participating in a clinical trial for MS, the method comprising:
(a) detecting at least one biomarker in a biological sample comprising CSF from the subject; and
(b) identifying the subject expressing the at least one biomarker in the biological sample, thereby identifying a patient as being suitable for participating in a clinical trial for MS;
wherein the at least one biomarker is chosen from CXCL13, CXCL10, CD27, NEFL, CCL4, and CCL3.

12. A method of diagnosing a subject as having MS, the method comprising:
(a) detecting at least one biomarker in a biological sample comprising CSF from the subject; and
(b) identifying the subject expressing the at least one biomarker in the biological sample as having MS;
wherein the at least one biomarker is chosen from CXCL13, CXCL10, CD27, NEFL, CCL4, and CCL3.

13. A method of identifying a subject having MS as expressing at least one biomarker in a biological sample comprising CSF, the method comprising:
(a) detecting the at least one biomarker in the biological sample; and
(b) identifying the subject having MS expressing the at least one biomarker in the biological sample;
wherein the at least one biomarker is chosen from CXCL13, CXCL10, CD27, NEFL, CCL4, and CCL3.

14. A method of identifying a subject as having an increased likelihood of developing MS, the method comprising:
(a) detecting at least one biomarker in a biological sample comprising CSF from a subject; and
(b) identifying a subject expressing the at least one biomarker in the biological sample, as having an increased likelihood of developing MS;
wherein the at least one biomarker is chosen from CXCL13, CXCL10, CD27, NEFL, CCL4, and CCL3.

15. A method of identifying a subject as likely to develop MS, the method comprising:
(a) detecting at least one biomarker in a biological sample comprising CSF from a subject; and
(b) identifying a subject expressing the at least one biomarker in the biological sample, as having an increased likelihood of developing MS;
wherein the at least one biomarker is chosen from CXCL13, CXCL10, CD27, NEFL, CCL4, and CCL3.

16. A method of monitoring progression of MS in a subject over time, the method comprising:
(a) detecting at least one biomarker in a first biological sample obtained from a subject at a first time point;
(b) detecting the at least one biomarker in a second biological sample obtained from the subject at a second time point; and
(c) identifying:
(i) a subject having increased at least one biomarker at the second time point, as compared to the at least one biomarker at the first time point, as having progressing MS, or
(ii) a subject having about the same or a decreased at least one biomarker at the second time point, as compared to the at least one biomarker at the first time point, as having static or regressing MS;
wherein the at least one biomarker is chosen from CXCL13, CXCL10, CD27, NEFL, CCL4, and CCL3.

17. A method of assessing the efficacy of a treatment of a pharmaceutically effective amount of tolebrutinib in a subject having MS, the method comprising:
(a) detecting (i) at least one biomarker in a first biological sample comprising CSF obtained from the subject at a first time point and (ii) the at least one biomarker in a second biological sample comprising CSF obtained from the subject at a second time point, wherein the subject is administered one or more doses of the treatment between the first and second time points; and
(b) determining a correlation between efficacy of the treatment and the at least one biomarker in the second biological sample as compared to the at least one biomarker in a sample obtained from an untreated patient, wherein the at least one biomarker in the second biological sample is about the same or decreased as compared to the abundance in the sample from the untreated patient, thereby indicating that the treatment is effective for MS in the subject;
wherein the at least one biomarker is chosen from CXCL13, CXCL10, CD27, NEFL, CCL4, and CCL3.

18. A method of treating a subject having MS, the method comprising:
(a) detecting CD27 in a biological sample comprising CSF from the subject;
(b) identifying the subject expressing CD27 in the biological sample as having MS; and
(c) administering a pharmaceutically effective amount of tolebrutinib to the subject.

19. A method of identifying a patient as being suitable for participating in a clinical trial for MS, the method comprising:
(a) detecting CD27 in a biological sample comprising CSF from the subject; and
(b) identifying the subject expressing CD27 in the biological sample, thereby identifying a patient as being suitable for participating in a clinical trial for MS.

20. A method of monitoring progression of MS in a subject over time, the method comprising:
(a) detecting CD27 in a first biological sample obtained from a subject at a first time point;
(b) detecting CD27 in a second biological sample obtained from the subject at a second time point; and
(c) identifying:
(i) a subject having increased CD27 at the second time point, as compared to CD27 at the first time point, as having progressing MS, or
(ii) a subject having about the same or a decreased CD27 at the second time point, as compared to CD27 at the first time point, as having static or regressing MS.

21. A method of assessing the efficacy of a treatment of a pharmaceutically effective amount of tolebrutinib in a subject having MS, the method comprising:
(a) detecting (i) CD27 in a first biological sample comprising CSF obtained from the subject at a first time point and (ii) CD27 in a second biological sample comprising CSF obtained from the subject at a second time point, wherein the subject is administered one or more doses of the treatment between the first and second time points; and
(b) determining a correlation between efficacy of the treatment and CD27 in the second biological sample as compared to CD27 in a sample obtained from an untreated patient, wherein the CD27 in the second biological sample is about the same or decreased as compared to the abundance in the sample from the untreated patient, thereby indicating that the treatment is effective for MS in the subject.

22. A method of treating a subject having MS, the method comprising:
(a) detecting NEFL in a biological sample comprising CSF from the subject;
(b) identifying the subject expressing NEFL in the biological sample as having MS; and
(c) administering a pharmaceutically effective amount of tolebrutinib to the subject.

23. A method of identifying a patient as being suitable for participating in a clinical trial for MS, the method comprising:
(a) detecting NEFL in a biological sample comprising CSF from the subject; and
(b) identifying the subject expressing NEFL in the biological sample, thereby identifying a patient as being suitable for participating in a clinical trial for MS.

24. A method of monitoring progression of MS in a subject over time, the method comprising:
(a) detecting NEFL in a first biological sample obtained from a subject at a first time point;
(b) detecting NEFL in a second biological sample obtained from the subject at a second time point; and
(c) identifying:
(i) a subject having increased NEFL at the second time point, as compared to NEFL at the first time point, as having progressing MS, or
(ii) a subject having about the same or a decreased NEFL at the second time point, as compared to NEFL at the first time point, as having static or regressing MS.

25. A method of assessing the efficacy of a treatment of a pharmaceutically effective amount of tolebrutinib in a subject having MS, the method comprising:
(a) detecting (i) NEFL in a first biological sample comprising CSF obtained from the subject at a first time point and (ii) NEFL in a second biological sample comprising CSF obtained from the subject at a second time point, wherein the subject is administered one or more doses of the treatment between the first and second time points; and
(b) determining a correlation between efficacy of the treatment and NEFL in the second biological sample as compared to NEFL in a sample obtained from an untreated patient, wherein the NEFL in the second biological sample is about the same or decreased as compared to the abundance in the sample from the untreated patient, thereby indicating that the treatment is effective for MS in the subject.

26. A method of treating a subject having MS, the method comprising:
(a) detecting CCL4 in a biological sample comprising CSF from the subject;
(b) identifying the subject expressing CCL4 in the biological sample as having MS; and
(c) administering a pharmaceutically effective amount of tolebrutinib to the subject.

27. A method of identifying a patient as being suitable for participating in a clinical trial for MS, the method comprising:
(a) detecting CCL4 in a biological sample comprising CSF from the subject; and
(b) identifying the subject expressing CCL4 in the biological sample, thereby identifying a patient as being suitable for participating in a clinical trial for MS.

28. A method of identifying a subject as likely to develop MS, the method comprising:
(a) detecting CCL4 in a biological sample comprising CSF from a subject; and
(b) identifying a subject expressing CCL4 in the biological sample, as having an increased likelihood of developing MS.

29. A method of monitoring progression of MS in a subject over time, the method comprising:
(a) detecting CCL4 in a first biological sample obtained from a subject at a first time point;
(b) detecting CCL4 in a second biological sample obtained from the subject at a second time point; and
(c) identifying:
(i) a subject having increased CCL4 at the second time point, as compared to CCL4 at the first time point, as having progressing MS, or
(ii) a subject having about the same or a decreased CCL4 at the second time point, as compared to CCL4 at the first time point, as having static or regressing MS.

30. A method of assessing the efficacy of a treatment of a pharmaceutically effective amount of tolebrutinib in a subject having MS, the method comprising:
(a) detecting (i) CCL4 in a first biological sample comprising CSF obtained from the subject at a first time point and (ii) CCL4 in a second biological sample comprising CSF obtained from the subject at a second time point, wherein the subject is administered one or more doses of the treatment between the first and second time points; and
(b) determining a correlation between efficacy of the treatment and CCL4 in the second biological sample as compared to CCL4 in a sample obtained from an untreated patient, wherein the CCL4 in the second biological sample is about the same or decreased as compared to the abundance in the sample from the untreated patient, thereby indicating that the treatment is effective for MS in the subject.

31. A method of treating a subject having MS, the method comprising:
(a) detecting CCL3 in a biological sample comprising CSF from the subject;
(b) identifying the subject expressing CCL3 in the biological sample as having MS; and
(c) administering a pharmaceutically effective amount of tolebrutinib to the subject.

32. A method of identifying a patient as being suitable for participating in a clinical trial for MS, the method comprising:
(a) detecting CCL3 in a biological sample comprising CSF from the subject; and
(b) identifying the subject expressing CCL3 in the biological sample, thereby identifying a patient as being suitable for participating in a clinical trial for MS.

33. A method of monitoring progression of MS in a subject over time, the method comprising:
(a) detecting CCL3 in a first biological sample obtained from a subject at a first time point;
(b) detecting CCL3 in a second biological sample obtained from the subject at a second time point; and
(c) identifying:
(i) a subject having increased CCL3 at the second time point, as compared to CCL3 at the first time point, as having progressing MS, or
(ii) a subject having about the same or a decreased CCL3 at the second time point, as compared to CCL3 at the first time point, as having static or regressing MS.

34. A method of assessing the efficacy of a treatment of a pharmaceutically effective amount of tolebrutinib in a subject having MS, the method comprising:
(a) detecting (i) CCL3 in a first biological sample comprising CSF obtained from the subject at a first time point and (ii) CCL3 in a second biological sample comprising CSF obtained from the subject at a second time point, wherein the subject is administered one or more doses of the treatment between the first and second time points; and
(b) determining a correlation between efficacy of the treatment and CCL3 in the second biological sample as compared to CCL3 in a sample obtained from an untreated patient, wherein the CCL3 in the second biological sample is about the same or decreased as compared to the abundance in the sample from the untreated patient, thereby indicating that the treatment is effective for MS in the subject.


SEQUENCE LISTING (13 sequences)

SEQ ID NO: 1 - protein, 10 aa, synthetic construct
  REGION 1..10: note = Synthetic: CDR L1 sequence
RASSSVSYMH

SEQ ID NO: 2 - protein, 7 aa, synthetic construct
  REGION 1..7: note = Synthetic: CDR L2 sequence
APSNLAS

SEQ ID NO: 3 - protein, 9 aa, synthetic construct
  REGION 1..9: note = Synthetic: CDR L3 sequence
QQWSFNPPT

SEQ ID NO: 4 - protein, 10 aa, synthetic construct
  REGION 1..10: note = Synthetic: CDR H1 sequence
GYTFTSYNMH

SEQ ID NO: 5 - protein, 17 aa, synthetic construct
  REGION 1..17: note = Synthetic: CDR H2 sequence
AIYPGNGDTSYNQKFKG

SEQ ID NO: 6 - protein, 13 aa, synthetic construct
  REGION 1..13: note = Synthetic: CDR H3 sequence
VVYYSNSYWYFDV

SEQ ID NO: 7 - protein, 107 aa, synthetic construct
  REGION 1..107: note = Synthetic: Ocrelizumab variable light chain sequence
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 8 - protein, 122 aa, synthetic construct [sequence omitted]

SEQ ID NO: 9 - protein, 213 aa, synthetic construct
  REGION 1..213: note = Synthetic: Ocrelizumab light chain amino acid sequence
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 452 aa, synthetic construct
  REGION 1..452: note = Synthetic: Ocrelizumab heavy chain amino acid sequence
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 451 aa, synthetic construct
  REGION 1..451: note = Synthetic: Ocrelizumab heavy chain amino acid sequence
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 213 aa, synthetic construct
  REGION 1..213: note = Synthetic: Rituximab light chain amino acid sequence
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 451 aa, synthetic construct
  REGION 1..451: note = Synthetic: Rituximab heavy chain amino acid sequence
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK